SEQUENCE LISTING (125 sequences)

SEQ ID NO: 1 - DNA, 2940 bp, Solanum bulbocastanum
  CDS (1)..(2940)
atggctgaagctttcattcaagttgtgctagacaatctcacttctttcctcaaaggggaacttgtattgcttttcggttttcaagatgagttccaaaggctttcaagcatgttttctacaatccaagccgtccttgaagatgctcaagagaagcaactcaacgacaagcctctagaaaattggttgcaaaaactcaatgctgctacatatgaagtcgatgacatcttggatgaatataaaactaaggccacaagattcttgcagtctgaatatggccgttatcatccaaaggttatccctttccgccacaaggttgggaaaaggatggatcaagtgatgaaaaaactgaatgcawttgctgaggaacgaaagaagtttcatttgcaagaaaagattatagagagacaagctgctacacgggaaacaggttctgtgttaactgaaccacaagtttatggaagggacaaagaaaaagatgagatagtgaaaatcctaataaacactgctagtgatgcccaaaaactctcagtcctcccaatacttggtatggggggactaggaaagacgactctttcccaaatggtcttcaatgatcagagagtaactgagcgtttctatcccaaaatatggatttgcatctcggatgattttaatgagaagaggttgataaaggcaatagtagaatctattgaagggaagtccctcagtgacatggacttggctccacttcaaaagaagcttcaagagttgctgaatggaaaaagatacttccttgtcttagatgatgtttggaatgaagatcaacataagtgggctaatttaagagcagtcttgaaggttggagcaagtggtgcatttgttctaactactactcgtcttgaaaaggttggatcaattatgggaacattgcaaccatatgaattgtcaaatctgtctccagaggattgttggtttttgttcatgcagcgtgcatttggacaccaagaagaaataaatccaaaccttatggcaatcggaaaggagattgtgaaaaaatgtggtggtgtgcctctagcagccaagactcttggaggtattttgcgcttcaagagagaagaaagagaatgggaacatgtgagagacagtccgatttggaatttgcctcaagatgaaagttctattctgcctgccctgaggcttagttaccatcatcttccacttgatttgagacaatgctttgtgtattgtgcggtattcccgaaggacaccaaaatggcaaaggaaaatcttatcgctttctggatggcacacggttttcttttatcgaaaggaaatttggagctagaggatgtaggtaatgaagtatggaatgaattatacttgaggtctttcttccaagagattgaagttgaatctggtaaaacttatttcaagatgcatgacctcatccatgatttggctacatctctgttttcagcaaacacatcaagcagcaatattcgtgaaataaatgctaattatgatggatatatgatgtcgattggttttgctgaagtggtatcttcttactctccgtcactcttgcaaaagtttgtctcattaagggtgcttaatctaagaaactcgaacctaaatcaattaccatcttccattggagatctagtacatttaagatacctggacttgtctggcaattttagaattcgtaatcttccaaagagattatgcaagcttcaaaatctgcagactcttgatctacattattgcgactctctttcttgtttgccaaaacaaacaagtaaacttggtagtctccgaaatcttttacttgatggctgttcattgacatcaacgccaccaaggataggattgttgacatgccttaagtctctaagttgctttgttattggcaagagaaaaggtcatcaacttggtgaactaaaaaacctaaatctctatggctcaatttcaatcacaaaacttgacagagtgaagaaagatacggatgcaaaagaagctaatttatctgctaaagcaaatctgcactctttatgcctgagttgggatcttgatggaaaacatagatatgattcagaagttcttgaagccctcaaaccacactccaatctgaaatatttagaaatcaatggcttcggaggaatccgtctcccagattggatgaatcaatcagttttgaaaaatgttgtctctattagaattagaggttgtgaaaactgctcatgcttaccaccctttggtgagctgccttgtctagaaagtctagagttacacaccgggtcagcggatgtggagtatgttgaagataatgttcatcctggaaggtttccatccttgaggaaacttgttatatgggactttagtaatctaaaaggattgctgaaaatggaaggagaaaagcaattccctgtgcttgaagagatgacattttactggtgccctatgtttgttattccgaccctttcttctgtcaagacattgaaagttattgtgacagatgcaacagttttgaggtccatatctaatcttagggctcttacttctctcgacattagcgataacgtagaagctacttcactcccagaagagatgttcaaaagccttgcaaatctcaaatacttgaaaatctctttctttaggaatctcaaagagttgcctaccagcttggctagtctcaatgctttgaagagcctcaaatttgaattttgtgacgcactagagagtctcccagaggaaggggtgaaaggtttaacttcactcaccgagttgtctgtcagtaactgtatgatgctaaaatgtttaccggagggattgcagcacctaacagccctcacaactttaacaattactcaatgtccaatagtattcaagcggtgtgagaggggaataggagaagactggcacaaaattgctcacattccatatttgactctatatgagtga

SEQ ID NO: 2 - protein, 979 aa, Solanum bulbocastanum
  misc_feature (119)..(119): The ′Xaa′ at location 119 stands for Ile, or Phe.
MAEAFIQVVLDNLTSFLKGELVLLFGFQDEFQRLSSMFSTIQAVLEDAQEKQLNDKPLENWLQKLNAATYEVDDILDEYKTKATRFLQSEYGRYHPKVIPFRHKVGKRMDQVMKKLNAXAEERKKFHLQEKIIERQAATRETGSVLTEPQVYGRDKEKDEIVKILINTASDAQKLSVLPILGMGGLGKTTLSQMVFNDQRVTERFYPKIWICISDDFNEKRLIKAIVESIEGKSLSDMDLAPLQKKLQELLNGKRYFLVLDDVWNEDQHKWANLRAVLKVGASGAFVLTTTRLEKVGSIMGTLQPYELSNLSPEDCWFLFMQRAFGHQEEINPNLMAIGKEIVKKCGGVPLAAKTLGGILRFKREEREWEHVRDSPIWNLPQDESSILPALRLSYHHLPLDLRQCFVYCAVFPKDTKMAKENLIAFWMAHGFLLSKGNLELEDVGNEVWNELYLRSFFQEIEVESGKTYFKMHDLIHDLATSLFSANTSSSNIREINANYDGYMMSIGFAEVVSSYSPSLLQKFVSLRVLNLRNSNLNQLPSSIGDLVHLRYLDLSGNFRIRNLPKRLCKLQNLQTLDLHYCDSLSCLPKQTSKLGSLRNLLLDGCSLTSTPPRIGLLTCLKSLSCFVIGKRKGHQLGELKNLNLYGSISITKLDRVKKDTDAKEANLSAKANLHSLCLSWDLDGKHRYDSEVLEALKPHSNLKYLEINGFGGIRLPDWMNQSVLKNVVSIRIRGCENCSCLPPFGELPCLESLELHTGSADVEYVEDNVHPGRFPSLRKLVIWDFSNLKGLLKMEGEKQFPVLEEMTFYWCPMFVIPTLSSVKTLKVIVTDATVLRSISNLRALTSLDISDNVEATSLPEEMFKSLANLKYLKISFFRNLKELPTSLASLNALKSLKFEFCDALESLPEEGVKGLTSLTELSVSNCMMLKCLPEGLQHLTALTTLTITQCPIVFKRCERGIGEDWHKIAHIPYLTLYE

SEQ ID NO: 3 - DNA, 8569 bp, Solanum bulbocastanum
  exon (2345)..(2804)
  Intron (2805)..(3456)
  exon (3457)..(5836)
cgggatcctgtcacataaattgacacaaagggagtacttgttaatgttgtaattattggcgaacaataatgttgttgattatcactttctgaataagtgttgtgtcacttggaaaaaacaccaaatagaactattcatgttttttctttagtatatataaatatgatctttaacttaatttcagcagacagtcatgatctttaactttaaatgtgcacaagtagattgacaggcttgctaattgagtgtctgttataatcagtattaaatactctcaaggtaatagtatattccagacaaattttgtgttaccaaattaaatatatttctaaaactctcctcaaagtagttaatatacttttgagtgttgtatcatgtttttaatataaaatgttaaaatttagatgaaatttactttctagttaaattggtcaaagttgaaagaatttcaagtgaaaaagtttttaataatttgacttttatgctatatttttttaaagttgaacgactttttaataaaaaagaataataaaattatatgataatttttataatacaatggcctttatatgatgaaaaaaaaagaaagaaattagatgacaacaatgtccaaaaataatcttaaagaattacgatttatatataataaaattaaatttaaaatttgatgaaaaaatagagaaaagaggaagatgatgaagtgaaatgacgtggtggtgggtccatgtgacataaaaaaaaattctcttaaataatcctttcatactaatgataaattttttttttttttttttttttactaattgcgtatagagaaaaggaaaatggggcggtaattacaaagtagggaatcgaactttatcaacaagttgagagttcaagtaatcaaccaactaaactactaaaatttttctaattaatgataattgtaattcatttagcataaaaaatttcattgcacttacttttagagttttgaaaacagtacttcatctattctatattaattaaattttctatattaattaaatttgtgaggtaatacaaacttattaagaaaaatatttaaggacataatttaactcatatttttcactattgttttttgtgaaatcataaatataactttgtaaatagtgcaatttatctcctagaagcaaatttcaccaaagaaaagggcaaagatggaaaagaaactaaatattcatcttaaactttgaacaattcaattattttgaacaatgaaaaaaatctcaaaaattcaattaatatgaaatggagagagtaactttattttagaggcaaaaaattagtactccatccgttcacttttatttgtcatgttgcgcttttcgaaagtcaatttgactaatttttaaagctaaattagattacactaattcaatattttaaacagaaaaattagatattcaaaaactatacaaaaaatattatacattgcaattttttgcatatcaatatgataaaaaaatatattgtaaaatattagtcaaaatttttatagtttgactctaatcatgaaaagtataataattaatagtggacggaggaagtattgtctttccagatttgtggccatttttgggccaagggccattagcagttctcttcattttctacttctgtctcatattagatgggcatcttactaaaaatatttgtctcatattacttgattatttattaaatcaaaaagaattaattaattttttctcattttacccctacaattaatatagttttaaaagttttaaacaaattttgaagaatcaaaatttctttttgcaagagacttattaatataaacaaaggataaaataataaaatttgtcaatttattgacgatcacttaataatcatataaaatagaatatgtttatctaatatgagacggagaaaatatatcctaaaatatttttggacagatatgtgatattctaaccattcactagactatattatgcattttagccgccaatgacttatttcagctttaattaattaggaaagaggaaactgccaatgaggaagagtaggggcgtagttgctgtcgacgaaaaaaagataatactcactcttttcgatttttatttttatttatcacttttaacctatcatgtaaaaagataattatttttttcatgctttatccttagtattaaacaatttaatagggattattttgtaaaatatttatatgaataattgttttcgtaatgaatttgtccggtcaaacaatgataaataaaaacgaatgaagagagtagaaaacaaaacaaaagaacaagttgacaacttgagagattaaaagggtccaaaacgccttggattttgagattccatatgtgaaatttccatgaaataattgaatttgtattattacaagtcaaacttcccatttcattccaactagccatcttggtttcaaaattacacattcattcattcacagatctaatattcttaatagtgatttccacatatggctgaagctttcattcaagttctgctagacaatctcacttctttcctcaaaggggaacttgtattgcttttcggttttcaagatgagttccaaaggctttcaagcatgttttctacaattcaagccgtccttgaagatgctcaggagaagcaactcaacaacaagcctctagaaaattggttgcaaaaactcaatgctgctacatatgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggtactcatcttaaattagaattacaacaactaagtttatattcatttttttggcaattatgaaattcagaaaagggttaaatatactcatgtcctatcgtaaatagtgtaaatatacctctcgttgtactttcgatctgaatatacttgtcaaatctggcaagctcagaatcaaattatccaccccaacttttaaatactcgatatctttagaaatccacctgtctaactcatccactacccattccctttgctttgaattcttttctttacctataaacgtggaacactcgatccgttttgcttttcttaacaaagcagctcagagaaaagaggttttcttctattctgtttctctgtgtgctgcacttgggtccttaatcccattaaaaacagggcatgttaatcccaacgacggtagcctttcctgacagctgactgtaaattttgtctaacaaagaaaaaaaaagattagacatgtttttccttgtcattgattaggctggatttctttcagagtggaacataggggatatattggaccaaaagtagaatgggtatatatttaaagtatttctgatagaacaggagtatattgtgcgaaaatatcctctattttctgttgtctcctaatgagtttgaatgtaataatattctcatgtggacattgcttgcaccaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaacctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatgaaaaaagaaaagctaatctctctctggatggcgcatggttttcttttatcaaaaggaaacatggagctagaggatgtgggcgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgatatggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaagttatttgctattgtttctttgtttgtgagtctttttggttcctgccattgtgattgcatgtaatttttttctagggttgtttgtttgttgagtctctctctcattggatgtaattctcttttggtaacaaattaacaatctatttgtattatacgctttcagaatctattacttatttgtaattgtttctttgtttgtaaattgtgagtatcttattgtatggaattttctgattttattttgaaaacaaatcaataagatccatctgcattatactcccttcgtctcattttatgtgacactttttggatttcgagattctttgatcttaaatttttcatagatcttttaaacattttgagttatcaattattgtgattttagtattttttatgtagtttacaaatacataaaatttattttttttaaaaaaagaagatttcatgcgcaaattcccgatcaaacttaaattactagactctcgaaaaatgaaaagtgtcacataaattgagacagagggagtacttgttaatgttgtaattattggcgaacaataatgttggtgattatcactttctgaataaatgttgtgtcacgtggaaaaaacaccaaatagaagtattcatgcttttttagtatatataaacatgatttttaacttggtttcagcggatagtcatgacctttaactctgaatgtgcacaagtagatacttgtataaaattaaacaaattttataaaattatacaatatgacactgagagtaattgataccaattgcagtcgttgctgcttttcgattctctgtcattctctaggtaattgattttacagaaaagggccaaaaatatccctgaagtaccagaaaaggtctcaaaataccaaccatccacattttggtctaaaaatatccttctactcatccttttttgtctaaaattaccctttcatccacatttttgctcacttatacccttataacaactctctccttttttttaaaaaaatatttattatgtgtcattttcttattgaatgaaataaaaatccacctctattaattttttcccataatttatccaaatcaaaacaatatattttttcaagatccaaaaaatatatttttttaaatctagtaatttctattttctatagcttttttccaaaaaaaaaatggttgttttagataattaaaatatctttaaaagtactagtcatgccacaattatagggacataatatattaatataaatcctaaaatattttataataatattttattataaaatatattaatatattatgtccctgtaattgtggcatgactaatatttttaaaaatattttaattatctaaaacaattttttttggaaaaaagctacagaaaatagaaattactagatttaaaaaaatacattttttggatcttgaaaagatatattgttttgatttggataaattatgagaaaaaattaatagaggtggatttttatttcattcaataagaaaatgacatataataagaatttaaaaaaaaaaaagagagagggttgttagttgcaagggtataagtgtgcaaagtggtggatggaagggtaattttagaccaaaaagatgagtagaagggtatttttagatcaaagatggatggagggtatttttagatcttttcatgtacttcaggggtatttttggcccgattttatttgattctccctctctttttggttctggttgattgacaggcctgctaattgagtgtttgttgtaatcagtattaattactctcaaggtaatattatattccaaacaaattttgtgttaccaaattaaatatatttctaaaactatcctgaaggtagttaatatactttttagtgttgtatcatgtttttaatataaaatattaaaatttagatgaaatttactttctagttaaattggtcaaagttgtaagaatttcaagtgaaagagtttttaataatttcacttttatgctatatatttttaaagttgaacgactttttaataaaaaagaataataaaattatatgataatttttataatacaatggcctttatatgatgaaaaaaaagaaagaaattagatgacaacaatgtccaaaaataatcttaaagaattatgatttatatataataaaattaaatttaaaatttgatgaaaaaatagagagaagaagaagatgatgaagtggaattatgtggtggtgggtccatgtgacataataaaaaacaattctcttaaataatcctttcatactaatgataaaagaaaatatatatatatatatatatttctttttacaaattgtgaatagagaaaaggaaaatggggtagcaattacaaggtaggaaatcaaactttatcaacaagttgagagttcaagtaatcaactttatcatatccgaaacattccttccgctttgagttcttttctttatggatcccg

SEQ ID NO: 4 - DNA, 2913 bp, Solanum bulbocastanum
  CDS (1)..(2913)
atggctgaagctttcattcaagttctgctagacaatctcacttctttcctcaaaggggaacttgtattgcttttcggttttcaagatgagttccaaaggctttcaagcatgttttctacaattcaagccgtccttgaagatgctcaggagaagcaactcaacaacaagcctctagaaaattggttgcaaaaactcaatgctgctacatatgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaacctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatgaaaaaagaaaagctaatctctctctggatggcgcatggttttcttttatcaaaaggaaacatggagctagaggatgtgggcgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgatatggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaa

SEQ ID NO: 5 - protein, 970 aa, Solanum bulbocastanum
MAEAFIQVLLDNLTSFLKGELVLLFGFQDEFQRLSSMFSTIQAVLEDAQEKQLNNKPLENWLQKLNAATYEVDDILDEYKTKATRFSQSEYGRYHPKVIPFRHKVGKRMDQVMKKLKAIAEERKNFHLHEKIVERQAVRRETGSVLTEPQVYGRDKEKDEIVKILINNVSDAQHLSVLPILGMGGLGKTTLAQMVFNDQRVTEHFHSKIWICVSEDFDEKRLIKAIVESIEGRPLLGEMDLAPLQKKLQELLNGKRYLLVLDDVWNEDQQKWANLRAVLKVGASGASVLTTTRLEKVGSIMGTLQPYELSNLSQEDCWLLFMQRAFGHQEEINPNLVAIGKEIVKKSGGVPLAAKTLGGILCFKREERAWEHVRDSPIWNLPQDESSILPALRLSYHQLPLDLKQCFAYCAVFPKDAKMKKEKLISLWMAHGFLLSKGNMELEDVGDEVWKELYLRSFFQEIEVKDGKTYFKMHDLIHDLATSLFSANTSSSNIREINKHSYTHMMSIGFAEVVFFYTLPPLEKFISLRVLNLGDSTFNKLPSSIGDLVHLRYLNLYGSGMRSLPKQLCKLQNLQTLDLQYCTKLCCLPKETSKLGSLRNLLLDGSQSLTCMPPRIGSLTCLKTLGQFVVGRKKGYQLGELGNLNLYGSIKISHLERVKNDMDAKEANLSAKGNLHSLSMSWNNFGPHIYESEEVKVLEALKPHSNLTSLKIYGFRGIHLPEWMNHSVLKNIVSILISNFRNCSCLPPFGDLPCLESLELHWGSADVEYVEEVDIDVHSGFPTRIRFPSLRKLDIWDFGSLKGLLKKEGEEQFPVLEEMIIHECPFLTLSSNLRALTSLRICYNKVATSFPEEMFKNLANLKYLTISRCNNLKELPTSLASLNALKSLKIQLCCALESLPEEGLEGLSSLTELFVEHCNMLKCLPEGLQHLTTLTSLKIRGCPQLIKRCEKGIGEDWHKISHIPNVNIYI

SEQ ID NO: 6 - DNA, 3592 bp, Solanum bulbocastanum
  exon (1)..(411)
  Intron (412)..(649)
  exon (650)..(3592)
atggctgaagctttcattcaagttctgctagacaatctcacttctttcctcaaaggggaacttgtattgcttttcggttttcaagatgagttccaaaggctttcaagcatgttttctacaattcaagccgtccttgaagatgctcaggagaagcaactcaacaacaagcctctagaaaattggttgcaaaaactcaatgctgctacatatgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggtactcatcttaaattagtattacaacaactaagtttatattcatttttttggcaattatgaaattcagaaaagggttaaatatactcatgtcctatcgtaaatagtgtaaatatacctctcgttgtactttcgatctgaatatacttgtcaaatctggcaagctcagaatcaaattatccaccccaacttttaaatactcgatatctttagaaatccacctgtctaactcatccactacccattccctttgctttgaattcttttctttacctataaacgtggaacactcgatccgttttgcttttcttaacaaagcagctcagagaaaagaggttttcttctattctgtttctctgtgtgctgcacttgggtccttaatcccattaaaaacagggcatgttaatcccaacgacggtagcctttcctgacagctgactgtaaattttgtctaacaaagaaaaaaaaagattagacatgtttttccttgtcattgattaggctggatttctttcagagtggaacataggggatatattggaccaaaagtagaatgggtatatatttaaagtatttctgatagaacaggagtatattgtgcgaaaatatcctctattttctgttgtctcctaatgagtttgaatgtaataatattctcatgtggacattgcttgcaccaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaatctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatggaaaaagaaaagctaatctctctctggatggcgcatggttttcttttatcaaaaggaaacatggagctagaggatgtgggcgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgataaggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaa

SEQ ID NO: 7 - DNA, 2913 bp, Solanum bulbocastanum
  CDS (1)..(2913)
atggctgaagctttcattcaagttctgctagacaatctcacttctttcctcaaaggggaacttgtattgcttttcggttttcaagatgagttccaaaggctttcaagcatgttttctacaattcaagccgtccttgaagatgctcaggagaagcaactcaacaacaagcctctagaaaattggttgcaaaaactcaatgctgctacatatgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaatctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatggaaaaagaaaagctaatctctctctggatggcgcatggttttcttttatcaaaaggaaacatggagctagaggatgtgggcgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgataaggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaa

SEQ ID NO: 8 - protein, 970 aa, Solanum bulbocastanum
MAEAFIQVLLDNLTSFLKGELVLLFGFQDEFQRLSSMFSTIQAVLEDAQEKQLNNKPLENWLQKLNAATYEVDDILDEYKTKATRFSQSEYGRYHPKVIPFRHKVGKRMDQVMKKLKAIAEERKNFHLHEKIVERQAVRRETGSVLTEPQVYGRDKEKDEIVKILINNVSDAQHLSVLPILGMGGLGKTTLAQMVFNDQRVTEHFHSKIWICVSEDFDEKRLIKAIVESIEGRPLLGEMDLAPLQKKLQELLNGKRYLLVLDDVWNEDQQKWANLRAVLKVGASGASVLTTTRLEKVGSIMGTLQPYELSNLSQEDCWLLFMQRAFGHQEEINPNLVAIGKEIVKKSGGVPLAAKTLGGILCFKREERAWEHVRDSPIWNLPQDESSILPALRLSYHQLPLDLKQCFAYCAVFPKDAKMEKEKLISLWMAHGFLLSKGNMELEDVGDEVWKELYLRSFFQEIEVKDGKTYFKMHDLIHDLATSLFSANTSSSNIREINKHSYTHMMSIGFAEVVFFYTLPPLEKFISLRVLNLGDSTFNKLPSSIGDLVHLRYLNLYGSGMRSLPKQLCKLQNLQTLDLQYCTKLCCLPKETSKLGSLRNLLLDGSQSLTCMPPRIGSLTCLKTLGQFVVGRKKGYQLGELGNLNLYGSIKISHLERVKNDKDAKEANLSAKGNLHSLSMSWNNFGPHIYESEEVKVLEALKPHSNLTSLKIYGFRGIHLPEWMNHSVLKNIVSILISNFRNCSCLPPFGDLPCLESLELHWGSADVEYVEEVDIDVHSGFPTRIRFPSLRKLDIWDFGSLKGLLKKEGEEQFPVLEEMIIHECPFLTLSSNLRALTSLRICYNKVATSFPEEMFKNLANLKYLTISRCNNLKELPTSLASLNALKSLKIQLCCALESLPEEGLEGLSSLTELFVEHCNMLKCLPEGLQHLTTLTSLKIRGCPQLIKRCEKGIGEDWHKISHIPNVNIYI

SEQ ID NO: 9 - DNA, 2979 bp, Solanum bulbocastanum
  CDS (1)..(2979)
atggctgaagctttccttcaagttctgctagataatctcacttttttcatccaaggggaacttggattggtttttggtttcgagaaggagtttaaaaaactttcaagtatgttttcaatgatccaagctgtgctagaagatgctcaagagaagcaactgaagtacaaggcaataaagaactggttacagaaactcaatgttgctgcatatgaagttgatgacatcttggatgactgtaaaactgaggcagcaagattcaagcaggctgtattggggcgttatcatccacggaccatcactttctgttacaaggtgggaaaaagaatgaaagaaatgatggaaaaactagatgcaattgcagaggaacggaggaattttcatttagatgaaaggattatagagagacaagctgctagacggcaaacaggttttgttttaactgagccaaaagtttatggaagggaaaaagaggaggatgagatagtgaaaatcttgataaacaatgttagttattccgaagaagttccagtactcccaatacttggtatggggggactaggaaagacgactctagcccaaatggtcttcaatgatcaaagaattactgagcatttcaatctaaagatatgggtttgtgtctcagatgattttgatgagaagaggttgattaaggcaattgtagaatctattgaaggaaagtcactgggtgacatggacttggctcccctccagaaaaagcttcaggagttgttgaatggaaaaagatactttcttgttttggatgatgtttggaatgaagatcaagaaaagtgggataatcttagagcagtattgaagattggagctagtggtgcttcaattctaattactactcgtcttgaaaaaattggatcaattatgggaactttgcaactatatcagttatcaaatttgtctcaagaagattgttggttgttgttcaagcaacgtgcattttgccaccaaaccgaaacaagtcctaaacttatggaaatcggaaaggagattgtgaagaaatgtgggggtgtgcctctagcagccaaaactcttggaggccttttacgcttcaagagggaagaaagtgaatgggaacatgtgagagatagtgagatttggaatttacctcaagatgaaaattctgttttgcctgccctgaggctgagttatcatcatcttccacttgatttgagacaatgttttgcatattgcgcagtattcccaaaggacaccaaaatagaaaaggaatatctcatcgctctctggatggcacacagttttcttttatcaaaaggaaacatggagctagaggatgtgggcaatgaagtatggaatgaattatacttgaggtcttttttccaagagattgaagttaaatctggtaaaacttatttcaagatgcatgatctcatccatgatttggctacatctatgttttcagcaagcgcatcaagcagaagtatacgccaaataaatgtaaaagatgatgaagatatgatgttcattgtaacaaattataaagatatgatgtccattggtttctccgaagtggtgtcttcttactctccttcgctctttaaaaggtttgtctcgttaagggtgcttaatctaagtaactcagaatttgaacagttaccgtcttccgttggagatctagtacatttaagataccttgacctgtctggtaataaaatttgtagtcttccaaagaggttgtgcaagcttcaaaatctgcagactcttgatctatataattgccagtcactttcttgtttgccgaaacaaacaagtaagctttgtagtctccggaatcttgtacttgatcactgtccattgacttctatgccaccaagaataggattgttgacatgccttaagacactaggttactttgttgtaggcgagaggaaaggttatcaacttggtgaactacgaaatttaaacctccgtggtgcaatttcaatcacacatcttgagagagtgaaaaatgatatggaggcaaaagaagccaatttatctgcaaaagcaaatctacactctttaagcatgagttgggatagaccaaacagatatgaatccgaagaagttaaagtgcttgaagccctcaaaccacatcccaatctgaaatatttagaaatcattgacttctgtggattctgtctccctgactggatgaatcactcagttttgaaaaatgttgtctctattctaattagcggttgtgaaaactgctcgtgcttaccaccctttggtgagctgccttgtctagaaagtctggagttacaagacgggtctgtggaggtggagtatgttgaagattctggattcctgacaagaagaagatttccatccctgagaaaacttcatataggtggcttttgtaatctgaaaggattgcagagaatgaaaggagcagagcaattccccgtgcttgaagagatgaagatttcggattgccctatgtttgtttttccgaccctttcttctgtcaagaaattagaaatttggggggaggcagatgcaggaggtttgagctccatatctaatctcagcactcttacatccctcaagattttcagtaaccacacagtgacttcactactggaagagatgttcaaaaaccttgaaaatctcatatacttgagtgtctctttcttggagaatctcaaagagctgcctaccagcctggctagtctcaacaatttgaagtgtctggatattcgttattgttacgcactagagagtctccccgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaa

SEQ ID NO: 10 - protein, 992 aa, Solanum bulbocastanum
MAEAFLQVLLDNLTFFIQGELGLVFGFEKEFKKLSSMFSMIQAVLEDAQEKQLKYKAIKNWLQKLNVAAYEVDDILDDCKTEAARFKQAVLGRYHPRTITFCYKVGKRMKEMMEKLDAIAEERRNFHLDERIIERQAARRQTGFVLTEPKVYGREKEEDEIVKILINNVSYSEEVPVLPILGMGGLGKTTLAQMVFNDQRITEHFNLKIWVCVSDDFDEKRLIKAIVESIEGKSLGDMDLAPLQKKLQELLNGKRYFLVLDDVWNEDQEKWDNLRAVLKIGASGASILITTRLEKIGSIMGTLQLYQLSNLSQEDCWLLFKQRAFCHQTETSPKLMEIGKEIVKKCGGVPLAAKTLGGLLRFKREESEWEHVRDSEIWNLPQDENSVLPALRLSYHHLPLDLRQCFAYCAVFPKDTKIEKEYLIALWMAHSFLLSKGNMELEDVGNEVWNELYLRSFFQEIEVKSGKTYFKMHDLIHDLATSMFSASASSRSIRQINVKDDEDMMFIVTNYKDMMSIGFSEVVSSYSPSLFKRFVSLRVLNLSNSEFEQLPSSVGDLVHLRYLDLSGNKICSLPKRLCKLQNLQTLDLYNCQSLSCLPKQTSKLCSLRNLVLDHCPLTSMPPRIGLLTCLKTLGYFVVGERKGYQLGELRNLNLRGAISITHLERVKNDMEAKEANLSAKANLHSLSMSWDRPNRYESEEVKVLEALKPHPNLKYLEIIDFCGFCLPDWMNHSVLKNVVSILISGCENCSCLPPFGELPCLESLELQDGSVEVEYVEDSGFLTRRRFPSLRKLHIGGFCNLKGLQRMKGAEQFPVLEEMKISDCPMFVFPTLSSVKKLEIWGEADAGGLSSISNLSTLTSLKIFSNHTVTSLLEEMFKNLENLIYLSVSFLENLKELPTSLASLNNLKCLDIRYCYALESLPEEGLEGLSSLTELFVEHCNMLKCLPEGLQHLTTLTSLKIRGCPQLIKRCEKGIGEDWHKISHIPNVNIYI

SEQ ID NO: 11 - DNA, 2967 bp, Solanum bulbocastanum
  CDS (1)..(2967)
atggcggaagcttttcttcaagttctgctagaaaatctcacttctttcatcggagataaacttgtattgattttcggtttcgaaaaggaatgtgaaaagctgtcgagtgtgttttccacaattcaagctgtgcttcaagatgctcaggagaagcaattgaaggacaaggcaattgagaattggttgcagaaactcaattctgctgcctatgaagttgatgatatattgggcgaatgtaaaaatgaggcaataagatttgagcagtctcgattagggttttatcacccagggattatcaatttccgtcacaaaattgggagaaggatgaaagagataatggagaaactagatgcaatatctgaggaaagaaggaagtttcatttccttgaaaaaattacagagagacaagctgccgctgctacgcgtgaaacaggttttgtgttaactgaaccaaaagtctacggaagggacaaagaggaggatgagatagtgaaaattctgataaacaatgttaatgttgccgaagaacttccagtcttccctataattggtatggggggactaggaaagacgacacttgcccaaatgatcttcaacgatgagagagtaactaagcatttcaatcccaaaatatgggtttgtgtctcagatgattttgatgagaagaggttaattaagacaattataggaaatattgaaagaagttctcctcatgttgaggacttggcttcatttcagaagaagctccaggagttattgaatggaaaacgatacttgcttgtcttagatgatgtttggaatgatgatctagaaaagtgggctaagttaagagcagtcttaactgttggagcaagaggtgcttctattctagctactactcgtcttgaaaaggttggatcaattatgggaacgttgcaaccatatcatttgtcaaatttgtctccacatgatagtttacttttgtttatgcaacgcgcatttgggcaacaaaaagaagcaaatcctaatctagtggccattggaaaggagattgtgaagaaatgtggtggtgtgcctttagcagccaagactcttggtggtcttttacgcttcaagagagaagagagtgaatgggaacatgtgagagataatgagatttggagtctgcctcaagatgaaagttctattttgcctgctctaagactgagttatcatcaccttccacttgatttgagacaatgctttgcgtattgtgcagtattcccaaaggacaccaaaatgataaaggaaaatctcattactctctggatggcgcatggttttcttttatcaaagggaaacttggagctagaggatgtgggtaatgaagtatggaatgaattatacttgaggtctttcttccaagaaattgaagctaaatcgggtaatacttatttcaagatacatgatctaatccatgatttggctacatctctgttttcggcaagcgcatcatgcggcaatatccgcgaaataaatgtcaaagattataagcatacagtgtccattggtttcgctgcagtggtgtcttcttactctccttcgctcttgaaaaagtttgtctcgttaagggtgcttaatctaagttactcaaaacttgagcaattaccgtcttccattggagatctattacatttaagatacctggacctgtcttgcaataacttccgtagtcttccagagaggttgtgcaagcttcaaaatcttcagactcttgatgtacataattgctactcacttaattgtttgccaaaacaaacaagtaaacttagtagtctccgacatcttgttgttgatggctgtccattgacttctactccaccaaggataggattgttgacatgccttaagactctaggtttctttattgtgggaagcaagaaaggttatcaacttggtgaactgaaaaacctaaatctctgcggctcaatttcaatcacacaccttgagagagtgaagaacgatacggatgcagaagccaatttatctgcaaaagcaaatctgcaatctttaagcatgagttgggataacgatggaccaaacagatatgaatccaaagaagttaaagtgcttgaagcactcaaaccacaccccaatctgaaatatttagagatcattgccttcggaggattccgttttccaagctggataaatcactcagttttggagaaggtcatctctgttagaattaaaagctgcaaaaactgcttgtgcttaccaccctttggggagcttccttgtctagaaaatctagagttacaaaacggatctgcggaggtggagtatgttgaagaggatgatgtccattctagattctccacaagaagaagctttccatccctgaaaaaacttcgtatatggttctttcgcagtttgaaagggctgatgaaagaggaaggagaagagaaattccccatgcttgaagagatggcgattttatattgccctctgtttgtttttccaaccctttcttctgtcaagaaattagaagttcacggcaacacaaacactagaggtttgagctccatatctaatcttagcactcttacttccctccgcattggtgctaactacagagcgacttcactcccagaagagatgttcacaagtcttacaaatctcgaattcttgagtttctttgacttcaagaatctcaaagatctgcctaccagcctgactagtctcaatgctttgaagcgtctccaaattgaaagttgtgactcactagagagtttccctgaacaagggctagaaggtttaacttcactcacacagttgtttgttaaatactgtaagatgctaaaatgtttacccgagggattgcagcacctaacagccctcacaaatttaggagtttctggttgtccagaagtggaaaagcgctgtgataaggaaataggagaagactggcacaaaattgctcacattccaaatctggatattcattag

SEQ ID NO: 12 - protein, 988 aa, Solanum bulbocastanum
MAEAFLQVLLENLTSFIGDKLVLIFGFEKECEKLSSVFSTIQAVLQDAQEKQLKDKAIENWLQKLNSAAYEVDDILGECKNEAIRFEQSRLGFYHPGIINFRHKIGRRMKEIMEKLDAISEERRKFHFLEKITERQAAAATRETGFVLTEPKVYGRDKEEDEIVKILINNVNVAEELPVFPIIGMGGLGKTTLAQMIFNDERVTKHFNPKIWVCVSDDFDEKRLIKTIIGNIERSSPHVEDLASFQKKLQELLNGKRYLLVLDDVWNDDLEKWAKLRAVLTVGARGASILATTRLEKVGSIMGTLQPYHLSNLSPHDSLLLFMQRAFGQQKEANPNLVAIGKEIVKKCGGVPLAAKTLGGLLRFKREESEWEHVRDNEIWSLPQDESSILPALRLSYHHLPLDLRQCFAYCAVFPKDTKMIKENLITLWMAHGFLLSKGNLELEDVGNEVWNELYLRSFFQEIEAKSGNTYFKIHDLIHDLATSLFSASASCGNIREINVKDYKHTVSIGFAAVVSSYSPSLLKKFVSLRVLNLSYSKLEQLPSSIGDLLHLRYLDLSCNNFRSLPERLCKLQNLQTLDVHNCYSLNCLPKQTSKLSSLRHLVVDGCPLTSTPPRIGLLTCLKTLGFFIVGSKKGYQLGELKNLNLCGSISITHLERVKNDTDAEANLSAKANLQSLSMSWDNDGPNRYESKEVKVLEALKPHPNLKYLEIIAFGGFRFPSWINHSVLEKVISVRIKSCKNCLCLPPFGELPCLENLELQNGSAEVEYVEEDDVHSRFSTRRSFPSLKKLRIWFFRSLKGLMKEEGEEKFPMLEEMAILYCPLFVFPTLSSVKKLEVHGNTNTRGLSSISNLSTLTSLRIGANYRATSLPEEMFTSLTNLEFLSFFDFKNLKDLPTSLTSLNALKRLQIESCDSLESFPEQGLEGLTSLTQLFVKYCKMLKCLPEGLQHLTALTNLGVSGCPEVEKRCDKEIGEDWHKIAHIPNLDIH

SEQ ID NO: 13 - DNA, 1920 bp, Solanum bulbocastanum
  CDS (1)..(1920)
atggctgaagctttcattcaagttgtgctagacaatctcacttctttcctcaaaggggaacttgtattgcttttcggttttcaagatgagttccaaaggctttcaagcatgttttctacaatccaagccgtccttgaagatgctcaagagaagcaactcaacgacaagcctctagaaaattggttgcaaaaactcaatgctgctacatatgaagtcgatgacatcttggatgaatataaaactaaggccacaagattcttgctgtctgaatatggccgttatcatccaaaggttatccctttccgtcacaaggttgggaaaaggatggaccaagtgatgaaaaaactgaatgcaattgctgaggaacgaaagaattttcatttgcaagaaaagattatagagagacaagctgctacacgggaaacaggttctgtgttaactgaatcacaagtttatggaagggacaaagaaaaagatgagatagtgaaaatcctaacaaacactgctagtgatgcccaaaaactctcagtcctcccaatacttggtatggggggactaggaaagacgactctttcccaaatggtcttcaatgatcagagagtaactgagcgtttctatcccaaaatatggatttgcgtctcggatgattttaatgagaagaggttgataaaggcaatagtagaatctattgaagggaagtccctcagtgacatggacttggctccacttcaaaagaagcttcaagagttgctgaatggaaaaagatacttccttgtcttagatgatgtttggaatgaagatcaacataagtgggctaatttaagagcagtcttgaaggttggagcaagtggtgcatttgttctaacaactactcgtcttgaaaaggttggatcaattatgggaacattgcaaccatatgaattgtcaaatctgtctccagaggattgttggtttttgttcatgcagcgtgcatttggacaccaagaagaaataaatccaaaccttgtggcaatcggaaaggagattgtgaaaaaatgtggtggtgtgcctctagcagccaagactcttggaggtattttgcgcttcaagagagaagaaagagaatgggaacatgtgagagacagtccgatttggaatttgcctcaagatgaaagttctattctgcctgccctgaggcttagttaccatcatcttccacttgatttgagacaatgctttgtgtattgtgcggtattcccaaaggacaccaaaatggcaaaggaaaatcttatcgctttttggatggcacatggttttcttttatcgaaaggaaatttggagctagaggatgtaggtaatgaagtatggaatgaattatacttgaggtctttcttccaagagattgaagttgaatctggtaaaacttatttcaagatgcatgacctcatccatgatttggctacgtctctgttttcagcaaacacatcaagcagcaatattcgtgaaataaatgctaattatgatggatatatgatgtcgattggtttcgctgaagtggtgtcttcttactctccttcactcttgcaaaagtttgtctcattaagggtgcttaatctaagaaactcgaacctaaatcaattaccatcttccattggagatctagtacatttaagatacctggacttgtctggcaatgttagaattcgtagtcttccaaggagattatgcaagcttcaaaatctgcagactcttgatctacattattgcgactctctttcttgtttgccaaaacaaacaagtaaacttggtagtctccgaaatcttttacttgatggctgttcattgacgtcaacgccaccaaggataggattgttgacatgccttaagtctctaagttgctttgttattggcaagagaaaggttatcaacttggtgaactaa

SEQ ID NO: 14 - protein, 639 aa, Solanum bulbocastanum
MAEAFIQVVLDNLTSFLKGELVLLFGFQDEFQRLSSMFSTIQAVLEDAQEKQLNDKPLENWLQKLNAATYEVDDILDEYKTKATRFLLSEYGRYHPKVIPFRHKVGKRMDQVMKKLNAIAEERKNFHLQEKIIERQAATRETGSVLTESQVYGRDKEKDEIVKILTNTASDAQKLSVLPILGMGGLGKTTLSQMVFNDQRVTERFYPKIWICVSDDFNEKRLIKAIVESIEGKSLSDMDLAPLQKKLQELLNGKRYFLVLDDVWNEDQHKWANLRAVLKVGASGAFVLTTTRLEKVGSIMGTLQPYELSNLSPEDCWFLFMQRAFGHQEEINPNLVAIGKEIVKKCGGVPLAAKTLGGILRFKREEREWEHVRDSPIWNLPQDESSILPALRLSYHHLPLDLRQCFVYCAVFPKDTKMAKENLIAFWMAHGFLLSKGNLELEDVGNEVWNELYLRSFFQEIEVESGKTYFKMHDLIHDLATSLFSANTSSSNIREINANYDGYMMSIGFAEVVSSYSPSLLQKFVSLRVLNLRNSNLNQLPSSIGDLVHLRYLDLSGNVRIRSLPRRLCKLQNLQTLDLHYCDSLSCLPKQTSKLGSLRNLLLDGCSLTSTPPRIGLLTCLKSLSCFVIGKRKVINLVN

SEQ ID NO: 15 - DNA, 879 bp, Solanum bulbocastanum
  CDS (1)..(879)
atgtggacattgcttggaccaggttctgttttaactgaaccacaagtttatggaagggacaaagaaaaggacgagatagtgaaaatcctgataaacaatgttagtgatgctcaagaagtttcagtcctcccaatagttggtatggggggactaggaaagacgactcttgcccaaatggtcttcaatgatcagacagtaactgagcatttgtatccgaaaatatggatttgtgtctccaatgattttgatgagaagaggttaataaaggcaattgtagaatctattgaaggaaggccactacttggtgagatggacttggctccacttcaaaagaagcttcaagagttgcggatgtggagtatgttgaagaagtggatattgatgttcattctggatttccccacaagaataaggtttccatccttgaggaaacttgatatatgggactttggtagtctgaaaggattgctgaaaaaggaaggagaagagcaattccctgtgcttgaagagatggagattaaatggtgccctatgtttgttattccgaccctttcttctgtcaagaaattggtagttcgtggggacaagtcagatgcaataggtttcagttccatatctaatctcagggctcttacttccctcaatattaactttaacaaagaagctacttcactcccagaagagatgttcaaaagccttgcaaatctcaaatacttgaaaatctcttcctttaggaatctcaaagagctgcctaccagcctggctagtctcaatgctttgcagagtctgacaattgaacattgtgacgcactagagagtctcccagaagaaggggtgaaaggtttaacttcactcaccgagttgtctgtccaggactga

SEQ ID NO: 16 - protein, 292 aa, Solanum bulbocastanum
MWTLLGPGSVLTEPQVYGRDKEKDEIVKILINNVSDAQEVSVLPIVGMGGLGKTTLAQMVFNDQTVTEHLYPKIWICVSNDFDEKRLIKAIVESIEGRPLLGEMDLAPLQKKLQELRMWSMLKKWILMFILDFPTRIRFPSLRKLDIWDFGSLKGLLKKEGEEQFPVLEEMEIKWCPMFVIPTLSSVKKLVVRGDKSDAIGFSSISNLRALTSLNINFNKEATSLPEEMFKSLANLKYLKISSFRNLKELPTSLASLNALQSLTIEHCDALESLPEEGVKGLTSLTELSVQD

SEQ ID NO: 17 - DNA, 1362 bp, Solanum bulbocastanum
  CDS (1)..(1362)
atggctgaagctttcattcaagttctgttagacaatctcacttctttcctcaaaggggaacttgcattgcttttcggttttcaagatgagttccaaaggctttcaagcatgttttctacaattcaagccgtccttgaagatgctcaggagaagcaactcaacaacaagcctctagaaaattggttgcaaaaactcaatgctgctacatatgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaatctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatggaaaaagaaaagctaatctctctctggatggcgcatggttttcttttatcaaaaggaaacatggagctagaggatgtgggcgatgaagtatggaaagaattatag

SEQ ID NO: 18 - protein, 453 aa, Solanum bulbocastanum
MAEAFIQVLLDNLTSFLKGELALLFGFQDEFQRLSSMFSTIQAVLEDAQEKQLNNKPLENWLQKLNAATYEVDDILDEYKTKATRFSQSEYGRYHPKVIPFRHKVGKRMDQVMKKLKAIAEERKNFHLHEKIVERQAVRRETGSVLTEPQVYGRDKEKDEIVKILINNVSDAQHLSVLPILGMGGLGKTTLAQMVFNDQRVTEHFHSKIWICVSEDFDEKRLIKAIVESIEGRPLLGEMDLAPLQKKLQELLNGKRYLLVLDDVWNEDQQKWANLRAVLKVGASGASVLTTTRLEKVGSIMGTLQPYELSNLSQEDCWLLFMQRAFGHQEEINPNLVAIGKEIVKKSGGVPLAAKTLGGILCFKREERAWEHVRDSPIWNLPQDESSILPALRLSYHQLPLDLKQCFAYCAVFPKDAKMEKEKLISLWMAHGFLLSKGNMELEDVGDEVWKEL

SEQ ID NO: 19 - DNA, 3096 bp, Solanum bulbocastanum
  CDS (1)..(3096)
atgtggctatccacttttccaactgcactggcagttgctgtttcagtcccttcctctcttactccacaggatttcagctggagaaagtttaaattgaagagaaatgtgaagatttcaagaaattttgaatttgctatcagttgttctggtgatagagctgcttcaattgggtttgatgtgccattccctaaggactacactgaattacttcaacaagtattcattctatttgctttttctccccttaaaattgggggtgacggtgagggtggaatagaaatgactggaagtatacaactcatacgtgagttctgtgatctcttggtaatacctgagaaagccacaaagaccagaatttttttcccggaggctaatgaagtgaaatttgcaagacaatcaatttttggaggagcatcttttaagttggactatttgacaaagccttcttttttcgaggatttcggtttcactgaaaaggtcaagatggctgaccgtgtcaagccagaagatgaactctttatagtcgcctatccatattttaatgtcaatggggaacttggattggtttttggtttcgagaaggagtttaaaaaactttcaagtatgttttcaatgatccaagctgtgctagaagatgctcaagagaagcaactgaagtacaaggcaataaagaactggttacagaaactcaatgttgctgcatatgaagttgatgacatcttggatgactgtaaaactgaggcagcaagattcaagcaggctgtattggggcgttatcatccacggaccatcactttctgttacaaggtgggaaaaagaatgaaagaaatgatggaaaaactagatgcaattgcagaggaacggaggaattttcatttagatgaaaggattatagagagacaagctgctagacggcaaacaggttttgttttaactgagccaaaagtttatggaaaggaaaaagaggaggatgagatagtgaaaatcttgataaacaatgttagttattccaaagaagttccagtactcccaatacttggtatggggggactaggaaagacgactctagcccaaatggtcttcaatgatcaaagaattactgagcatttcaatctaaagatatgggtttgtgtctcagatgattttgatgagaagaggttgattaaggcaattgtagaatctattgaaggaaagtcactgggtgacatggacttggctcccctccagaaaaagcttcaggagttgttgaatggaaaaagatactttcttgttttggatgatgtttggaatgaagatcaagaaaagtgggataatcttagagcagtattgaagattggagctagtggtgcttcaattctaattactactcgtcttgaaaaaattggatcaattatgggaactttgcaactatatcagttatcaaatttgtctcaagaagattgttggttgttgttcaagcaacgtgcattttgccaccaaaccgaaacaagtcctaaacttatggaaatcggaaaggagattgtgaagaaatgtgggggtgtgcctctagcagccaaaactcttggaggccttttacgcttcaagagggaagaaagtgaatgggaacatgtgagagatagtgagatttggaatttacctcaagatgaaaattctgttttgcctgccctgaggctgagttatcatcatcttccacttgatttgagacaatgttttgcatattgcgcagtattcccaaaggacaccaaaatagaaaaggaatatctcatcgctctctggatggcacacagttttcttttatcaaaaggaaacatggagctagaggatgtgggcaatgaagtatggaatgaattatacttgaggtcttttttccaagagattgaagttaaatctgcaagcgcatcaagcagaagtatccgccaaataaatgtaaaagatgatgaagatatgatgttcattgtaacaaattataaagatatgatgtccattggttctccgaagtggtgtcttcttactctccttcgctctttaaaaggcgagaggaaaggttatcaacttggtgaactacgaaatttaaacctccgtggtgcaatttcaatcacacatcttgagagagtgaaaaacgatatggaggcaaaagaagccaatttatctgcaaaagcaaatctacactctttaagcatgagttgggatagaccaaacagatatgaatccgaagaagttaaagtgcttgaagccctcaaaccacatcccaatctgaaatatttagaaatcattgacttctgtggattctgtctccctgactggatgaatcactcagttttgaaaaatgttgtctctattctaattagcggttgtgaaaactgctcgtgcttaccaccctttggtgagctgccttgtctagaaagtctggagttacaagatgggtctgtggaggtggagtttgttgaagattctggattcccgacaagaagaagatttccatccctgagaaaacttcatataggtggcttttgtaatctgaaaggattgcagagaatggaaggagaagagcaattccccgtgcttgaagagatgaagatttcggattgccctatgtttgtttttccgaccctttcttctgtcaagaaattagaaatttggggggaggcagatgcaagaggtttgagctccatatctaatctcagcactcttacatccctcaaaattttcagtaaccacacagtgacttcactactggaagagatgttcaaaagcctcgaaaatctcaaatacttgagtgtctcttacttggagaatctcaaagagctgcctaccagcctggctagtctcaataatttgaagtgtctggatattcgttattgttacgcactagagagtctccccgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaacgtgaatatatatatttaa

SEQ ID NO: 20 - protein, 1031 aa, Solanum bulbocastanum
MWLSTFPTALAVAVSVPSSLTPQDFSWRKFKLKRNVKISRNFEFAISCSGDRAASIGFDVPFPKDYTELLQQVFILFAFSPLKIGGDGEGGIEMTGSIQLIREFCDLLVIPEKATKTRIFFPEANEVKFARQSIFGGASFKLDYLTKPSFFEDFGFTEKVKMADRVKPEDELFIVAYPYFNVNGELGLVFGFEKEFKKLSSMFSMIQAVLEDAQEKQLKYKAIKNWLQKLNVAAYEVDDILDDCKTEAARFKQAVLGRYHPRTITFCYKVGKRMKEMMEKLDAIAEERRNFHLDERIIERQAARRQTGFVLTEPKVYGKEKEEDEIVKILINNVSYSKEVPVLPILGMGGLGKTTLAQMVFNDQRITEHFNLKIWVCVSDDFDEKRLIKAIVESIEGKSLGDMDLAPLQKKLQELLNGKRYFLVLDDVWNEDQEKWDNLRAVLKIGASGASILITTRLEKIGSIMGTLQLYQLSNLSQEDCWLLFKQRAFCHQTETSPKLMEIGKEIVKKCGGVPLAAKTLGGLLRFKREESEWEHVRDSEIWNLPQDENSVLPALRLSYHHLPLDLRQCFAYCAVFPKDTKIEKEYLIALWMAHSFLLSKGNMELEDVGNEVWNELYLRSFFQEIEVKSASASSRSIRQINVKDDEDMMFIVTNYKDMMSIGSPKWCLLTLLRSLKGERKGYQLGELRNLNLRGAISITHLERVKNDMEAKEANLSAKANLHSLSMSWDRPNRYESEEVKVLEALKPHPNLKYLEIIDFCGFCLPDWMNHSVLKNVVSILISGCENCSCLPPFGELPCLESLELQDGSVEVEFVEDSGFPTRRRFPSLRKLHIGGFCNLKGLQRMEGEEQFPVLEEMKISDCPMFVFPTLSSVKKLEIWGEADARGLSSISNLSTLTSLKIFSNHTVTSLLEEMFKSLENLKYLSVSYLENLKELPTSLASLNNLKCLDIRYCYALESLPEEGLEGLSSLTELFVEHCNMLKCLPEGLQHLTTLTSLKIRGCPQLIKRCEKGIGEDWHKISHIPNVNIYI

SEQ ID NO: 21 - DNA, 2967 bp, Solanum bulbocastanum
  CDS (1)..(2967)
atggcggaagcttttcttcaagttctgctagaaaatctcacttctttcatcggagataaacttgtattgattttcggtttcgaaaaggaatgtgaaaagctgtcgagtgtgttttccacaattcaagctgtggttcaagatgctcaggagaagcaattgaaggacaaggcaattgagaattggttgcagaaactcaattctgctgcctatgaagttgatgatatattgggcgaatgtaaaaatgaggcaataagatttgagcagtctcgattagggttttatcacccagggattatcaatttccgtcacaaaattgggagaaggatgaaagagataatggagaaactagatgcaattgctgaggaaagaaggaagtttcatttccttgaaaaaattacggagagacaagctgccgctgctacgcgtgaaacaggttttgtgttaactgaaccaaaagtctacggaagggacaaagaggaggatgagatagtgaaaattctgataaacaatgttaatgttgccgaagaacttccagtcttccctataattggtatggggggactaggaaagacgacacttgcccaaatgatcttcaacgatgagagagtaactaagcatttcaatcccaaaatatgggtttgtgtctcagatgattttgatgagaagaggttaattaagacaattataggaaatattgaaagaagttctcctcatgttgaggacttggcttcatttcagaagaagctccaggagttattgaatggaaaacgatacttgcttgtcttagatgatgtttggaatgatgatctagaaaagtgggctaagttaagagcagtcttaactgttggagcaagaggtgcttctattctagctactactcgtcttgaaaaggttggatcaattatgggaacgtcgcaaccatatcatttgtcaaatttgtctccacatgatagtttacttttgtttatgcaacgcgcatttgggcaacaaaaagaagcaaatcctaatctagtggccattggaaaggagattgtgaagaaatgtggtggtgtgcctttagcggccaagactcttggtggtcttttacgcttcaagagagaagagagtgaatgggaacatgtgagagataatgagatttggagtctgcctcaagatgaaagttctattttgcctgctctaagactgagttatcatcaccttccacttgatttgagacaatgctttgcgtattgtgcagtattcccaaaggacaccaaaatgataaaggaaaatctcattactctctggatggcgcatggttttcttttatcaaagggaaacttggagctagaggatgtgggtaatgaagtatggaatgaattatacttgaggtctttcttccaagaaattgaagctaaatcgggtaatacttatttcaagatacatgatctaatccatgatttggctacatctctgttttcggcaagcgcatcatgcggcaatatccgcgaaataaatgtcaaagattataagcatacagtgtccattggtttctctgcagtggtgtcttcttactctccttcgctcttgaaaaagtttgtctcgttaagggtgcttaatctaagttactcaaaacttgagcaattaccgtcttccattggagatctattacatttaagatacctggacctgtcttgcaataacttccgtagtcttccagagaggttgtgcaagcttcaaaatcttcagactcttgatgtacataattgctactcacttaattgtttgccaaaacaaacaagtaaacttagtagtctccgacatcttgttgttgatggctgtccattgacttctactccaccaaggataggattgttgacatgccttaagactctaggtttctttattgtgggaagcaagaaaggttatcaacttggtgaactgaaaaacctaaatctctgcggctcaatttcaatcacacaccttgagagagtgaagaacgatacggatgcagaagccaatttatctgcaaaagcaaatctgcaatctttaagcatgagttgggataacgatggaccaaacagatatgaatccgaagaagttaaagtgcttgaagcactcaaaccacaccccaatctgaaatatttagagatcattgccttcggaggattccgttttccaagctggataaatcactcagttttggagaaggtcatctctgttagaattaaaagctgcaaaaactgcttgtgcttaccaccctttggggagcttccttgtctagaaaatctagagttacaaaacggatctgcggaggtggagtatgttgaagaggatgatgtccattctagattctccacaagaagaagctttccatccctgaaaaaacttcgtatatggttctttcgcagtttgaaagggctgatgaaagaggaaggagaagagaaattccccatgcttgaagagatggcgattttatattgccctctgtttgtttttccaaccctttcttctgtcaagaaattagaagttcacggcaacacaaacactagaggtttgagctccatatctaatcttagcactcttacttccctccgcattggtgctaactacagagcgacttcactcccagaagagatgttcacaagtcttacaaatctcgaattcttgagtttctttgacttcaagaatctcaaagatctgcctaccagcctgactagtctcaatgctttgaagcgtctccaaattgaaagttgtgactcactagagagtttccctgaacaagggctagaaggtttaacttcactcacacagttgtttgttaaatactgtaagatgctaaaatgtttacccgagggattgcagcacctaacagccctcacaaatttaggagtttctggttgtccagaagtggaaaagcgctgtgataaggaaataggagaagactggcacaaaattgctcacattccaaatctggatattcattag

SEQ ID NO: 22 - protein, 988 aa, Solanum bulbocastanum
MAEAFLQVLLENLTSFIGDKLVLIFGFEKECEKLSSVFSTIQAVVQDAQEKQLKDKAIENWLQKLNSAAYEVDDILGECKNEAIRFEQSRLGFYHPGIINFRHKIGRRMKEIMEKLDAIAEERRKFHFLEKITERQAAAATRETGFVLTEPKVYGRDKEEDEIVKILINNVNVAEELPVFPIIGMGGLGKTTLAQMIFNDERVTKHFNPKIWVCVSDDFDEKRLIKTIIGNIERSSPHVEDLASFQKKLQELLNGKRYLLVLDDVWNDDLEKWAKLRAVLTVGARGASILATTRLEKVGSIMGTSQPYHLSNLSPHDSLLLFMQRAFGQQKEANPNLVAIGKEIVKKCGGVPLAAKTLGGLLRFKREESEWEHVRDNEIWSLPQDESSILPALRLSYHHLPLDLRQCFAYCAVFPKDTKMIKENLITLWMAHGFLLSKGNLELEDVGNEVWNELYLRSFFQEIEAKSGNTYFKIHDLIHDLATSLFSASASCGNIREINVKDYKHTVSIGFSAVVSSYSPSLLKKFVSLRVLNLSYSKLEQLPSSIGDLLHLRYLDLSCNNFRSLPERLCKLQNLQTLDVHNCYSLNCLPKQTSKLSSLRHLVVDGCPLTSTPPRIGLLTCLKTLGFFIVGSKKGYQLGELKNLNLCGSISITHLERVKNDTDAEANLSAKANLQSLSMSWDNDGPNRYESEEVKVLEALKPHPNLKYLEIIAFGGFRFPSWINHSVLEKVISVRIKSCKNCLCLPPFGELPCLENLELQNGSAEVEYVEEDDVHSRFSTRRSFPSLKKLRIWFFRSLKGLMKEEGEEKFPMLEEMAILYCPLFVFPTLSSVKKLEVHGNTNTRGLSSISNLSTLTSLRIGANYRATSLPEEMFTSLTNLEFLSFFDFKNLKDLPTSLTSLNALKRLQIESCDSLESFPEQGLEGLTSLTQLFVKYCKMLKCLPEGLQHLTALTNLGVSGCPEVEKRCDKEIGEDWHKIAHIPNLDIH

SEQ ID NO: 23 - DNA, 2000 bp, Artificial Sequence
  Promoter
acgactttttaataaaaaagaataataaaattatatgataatttttataatacaatggcctttatatgatgaaaaaaaaagaaagaaattagatgacaacaatgtccaaaaataatcttaaagaattacgatttatatataataaaattaaatttaaaatttgatgaaaaaatagagaaaagaggaagatgatgaagtgaaatgacgtggtggtgggtccatgtgacataaaaaaaaattctcttaaataatcctttcatactaatgataaaattttttttttttttttttttttactaattgcgtatagagaaaaggaaaatggggcggtaattacaaagtagggaatcgaactttatcaacaagttgagagttcaagtaatcaaccaactaaactactaaaatttttctaattaatgataattgtaattcatttagcataaaaaatttcattgcacttacttttagagttttgaaaacagtacttcatctattctatattaattaaattttctatattaattaaatttgtgaggtaatacaaacttattaagaaaaatatttaaggacataatttaactcatatttttcactattgttttttgtgaaatcataaatataactttgtaaatagtgcaatttatctcctagaagcaaatttcaccaaagaaaagggcaaagatggaaaagaaactaaatattcatcttaaactttgaacaattcaattattttgaacaatgaaaaaaatctcaaaaattcaattaatatgaaatggagagagtaactttattttagaggcaaaaaattagtactccatccgttcacttttatttgtcatgttgcgcttttcgaaagtcaatttgactaatttttaaagctaaattagattacactaattcaatattttaaacagaaaaattagatattcaaaaactatacaaaaaatattatacattgcaattttttgcatatcaatatgataaaaaaatatattgtaaaatattagtcaaaatttttatagtttgactctaatcatgaaaagtataataattaatagtggacggaggaagtattgtctttccagatttgtggccatttttgggccaagggccattagcagttctcttcattttctacttctgtctcatattagatgggcatcttactaaaaatatttgtctcatattacttgattatttattaaatcaaaaagaattaattaattttttctcattttacccctacaattaatatagttttaaaagttttaaacaaattttgaagaatcaaaatttctttttgcaagagacttattaatataaacaaaggataaaataataaaatttgtcaatttattgacgatcacttaataatcatataaaatagaatatgtttatctaatatgagacggagaaaatatatcctaaaatatttttggacagatatgtgatattctaaccattcactagactatattatgcattttagccgccaatgacttatttcagctttaattaattaggaaagaggaaactgccaatgaggaagagtaggggcgtagttgctgtcgacgaaaaaaagataatactcactcttttcgatttttatttttatttatcacttttaacctatcatgtaaaaagataattatttttttcatgctttatccttagtattaaacaatttaatagggattattttgtaaaatatttatatgaataattgttttcgtaatgaatttgtccggtcaaacaatgataaataaaaatgaatgaagagagtagaaaacaaaacaaaagaacaagttgacaacttgagagattaaaagggtccaaaacgccttggattttgagattccatatgtgaaatttccatgaaataattgaatttgtattattacaagtcaaactttccatttcattccaactagccatcttggtttcaaaattacacattcattcattcacagatctaatattcttaatagtgatttccacat

SEQ ID NO: 24 - DNA, 32 bp, Artificial Sequence
  Primer
ctaccttgtaattaccgccccattttcctttt

SEQ ID NO: 25 - DNA, 32 bp, Artificial Sequence
  Primer
tgtcacataaattgacacaaagggagtacttg

SEQ ID NO: 26 - DNA, 32 bp, Artificial Sequence
  Primer
aagcggaaggaatggtttgggtatgataaaat

SEQ ID NO: 27 - DNA, 32 bp, Artificial Sequence
  Primer
ctttcaaaatgggaaaggagattaaaggtgga

SEQ ID NO: 28 - DNA, 33 bp, Artificial Sequence
  Primer
ccttgtaattgctaccccattagtatgaaagga

SEQ ID NO: 29 - DNA, 32 bp, Artificial Sequence
  Primer
aggagattaaaggtggataatcaaacctcacc

SEQ ID NO: 30 - DNA, 32 bp, Artificial Sequence
  Primer
gtttcttttccatctttgcccttttctttagg

SEQ ID NO: 31 - DNA, 32 bp, Artificial Sequence
  Primer
atcaattttatcatacccaaaccattccttcc

SEQ ID NO: 32 - DNA, 35 bp, Artificial Sequence
  Primer
ttgacttgagtacaacaaatccaagtataaagaag

SEQ ID NO: 33 - DNA, 32 bp, Artificial Sequence
  Primer
gtgaaggcaagtttagaaaatggtcatgatac

SEQ ID NO: 34 - DNA, 34 bp, Artificial Sequence
  Primer
tgattataacagacactcaattagcaagcctgtg

SEQ ID NO: 35 - DNA, 32 bp, Artificial Sequence
  Primer
ataaagaaaagaactcaaagcggaaggaatgg

SEQ ID NO: 36 - DNA, 10 bp, Artificial Sequence
  Primer
ggcactgagg

SEQ ID NO: 37 - DNA, 25 bp, Artificial Sequence
  Primer
cccttcttattctttaagcaaactt

SEQ ID NO: 38 - DNA, 19 bp, Artificial Sequence
  Primer
atttttcccctcccagctt

SEQ ID NO: 39 - DNA, 20 bp, Artificial Sequence
  Primer
attttgctcgaatcgctcat

SEQ ID NO: 40 - DNA, 21 bp, Artificial Sequence
  Primer
tcccaattggatcacttgtct

SEQ ID NO: 41 - DNA, 21 bp, Artificial Sequence
  Primer
tgtttgtgcattgaagattgg

SEQ ID NO: 42 - DNA, 21 bp, Artificial Sequence
  Primer
tggtaagaagggcattccata

SEQ ID NO: 43 - DNA, 23 bp, Artificial Sequence
  Primer
ggaaatactagaggggagggagt

SEQ ID NO: 44 - DNA, 22 bp, Artificial Sequence
  Primer
tgaataagcagttcggtttgaa

SEQ ID NO: 45 - DNA, 20 bp, Artificial Sequence
  Primer
tctcttgggatcacgattca

SEQ ID NO: 46 - DNA, 20 bp, Artificial Sequence
  Primer
tttaatttcggcggatgaac

SEQ ID NO: 47 - DNA, 23 bp, Artificial Sequence
  Primer
tttggaggatagcaatacttgga

SEQ ID NO: 48 - DNA, 23 bp, Artificial Sequence
  Primer
agcaactggtgagaaaatgtctt

SEQ ID NO: 49 - DNA, 20 bp, Artificial Sequence
  Primer
cggttcagctgacctttcat

SEQ ID NO: 50 - DNA, 20 bp, Artificial Sequence
  Primer
acctgcgagtggatcaaaac

SEQ ID NO: 51 - DNA, 24 bp, Artificial Sequence
  Primer
tcacaatgctaatatgtggtttga

SEQ ID NO: 52 - DNA, 20 bp, Artificial Sequence
  Primer
agttgtttgtggctgccatt

SEQ ID NO: 53 - DNA, 21 bp, Artificial Sequence
  Primer
aggtgtccaagtgaaaagtcg

SEQ ID NO: 54 - DNA, 19 bp, Artificial Sequence
  Primer
atcaagcacctccccaaac

SEQ ID NO: 55 - DNA, 20 bp, Artificial Sequence
  Primer
aactagcccgcgatcaacta

SEQ ID NO: 56 - DNA, 20 bp, Artificial Sequence
  Primer
aaaccgacacagatgcaaca

SEQ ID NO: 57 - DNA, 20 bp, Artificial Sequence
  Primer
ccctctgttccgtgacaaat

SEQ ID NO: 58 - DNA, 20 bp, Artificial Sequence
  Primer
cacagaagggggttgatctc

SEQ ID NO: 59 - DNA, 26 bp, Artificial Sequence
  Primer
tgagttccacagtctgtacataacaa

SEQ ID NO: 60 - DNA, 22 bp, Artificial Sequence
  Primer
tttcttcctctcccttctcctt

SEQ ID NO: 61 - DNA, 20 bp, Artificial Sequence
  Primer
aacaagatgagcctggtgtg

SEQ ID NO: 62 - DNA, 20 bp, Artificial Sequence
  Primer
atcacatcccagaggcaaaa

SEQ ID NO: 63 - DNA, 20 bp, Artificial Sequence
  Primer
atcaatccatcatgtgagca

SEQ ID NO: 64 - DNA, 22 bp, Artificial Sequence
  Primer
tcagaaaataagcacgttgaca

SEQ ID NO: 65 - DNA, 20 bp, Artificial Sequence
  Primer
cttgagaaggcaacgacaga

SEQ ID NO: 66 - DNA, 20 bp, Artificial Sequence
  Primer
gaaggcgggtaaacagacag

SEQ ID NO: 67 - DNA, 20 bp, Artificial Sequence
  Primer
caatcgctccttccaacttc

SEQ ID NO: 68 - DNA, 20 bp, Artificial Sequence
  Primer
tgagcagcattcgaagaaaa

SEQ ID NO: 69 - DNA, 24 bp, Artificial Sequence
  Primer
aggaatctcctcaagttctacaca

SEQ ID NO: 70 - DNA, 19 bp, Artificial Sequence
  Primer
gatacgggtgccaggattc

SEQ ID NO: 71 - DNA, 20 bp, Artificial Sequence
  Primer
ttcaaccagcaagttcaagc

SEQ ID NO: 72 - DNA, 20 bp, Artificial Sequence
  Primer
ttattgtccatgtcgctcca

SEQ ID NO: 73 - DNA, 20 bp, Artificial Sequence
  Primer
gttcccatgcctaaaccaga

SEQ ID NO: 74 - DNA, 20 bp, Artificial Sequence
  Primer
atcgcccgctcaacttaata

SEQ ID NO: 75 - DNA, 20 bp, Artificial Sequence
  Primer
tgaggtattgctgtgggttg

SEQ ID NO: 76 - DNA, 21 bp, Artificial Sequence
  Primer
tgaattcagcccagaagtgaa

SEQ ID NO: 77 - DNA, 20 bp, Artificial Sequence
  Primer
attcaatcgcctgtccaaac

SEQ ID NO: 78 - DNA, 20 bp, Artificial Sequence
  Primer
cattgctctcgttggatgaa

SEQ ID NO: 79 - DNA, 20 bp, Artificial Sequence
  Primer
ccctcgacatgaaccagaag

SEQ ID NO: 80 - DNA, 20 bp, Artificial Sequence
  Primer
tgtccatgtaggccaagacc

SEQ ID NO: 81 - DNA, 20 bp, Artificial Sequence
  Primer
acaggccagggttcaaaata

SEQ ID NO: 82 - DNA, 20 bp, Artificial Sequence
  Primer
gcaatggacagacttgatgc

SEQ ID NO: 83 - DNA, 21 bp, Artificial Sequence
  Primer
gaggcaaaccctctgttccgt

SEQ ID NO: 84 - DNA, 21 bp, Artificial Sequence
  Primer
gctccaagtggaggaaatgcc

SEQ ID NO: 85 - DNA, 20 bp, Artificial Sequence
  Primer
aacaagatgagcctggtgtg

SEQ ID NO: 86 - DNA, 20 bp, Artificial Sequence
  Primer
atcacatcccagaggcaaaa

SEQ ID NO: 87 - DNA, 18 bp, Artificial Sequence
  Primer
gcttagtgcccttaagcg

SEQ ID NO: 88 - DNA, 18 bp, Artificial Sequence
  Primer
ctgactaaccggatggcc

SEQ ID NO: 89 - DNA, 26 bp, Artificial Sequence
  Primer
aaaattgtcctcctctaattttcttt

SEQ ID NO: 90 - DNA, 23 bp, Artificial Sequence
  Primer
tgatatgaaaagaaagtggttgc

SEQ ID NO: 91 - DNA, 21 bp, Artificial Sequence
  Primer
cgtgaagtgaaatgctcaaca

SEQ ID NO: 92 - DNA, 20 bp, Artificial Sequence
  Primer
gcaaactttggaaggattcg

SEQ ID NO: 93 - DNA, 20 bp, Artificial Sequence
  Primer
cctgagcctcggtgagagta

SEQ ID NO: 94 - DNA, 20 bp, Artificial Sequence
  Primer
acccaaaactcccaacctct

SEQ ID NO: 95 - DNA, 20 bp, Artificial Sequence
  Primer
ctggtttgacaatgctggtg

SEQ ID NO: 96 - DNA, 20 bp, Artificial Sequence
  Primer
gacactcaaggctgccattt

SEQ ID NO: 97 - DNA, 21 bp, Artificial Sequence
  Primer
tctgcagaaaaccatctcagg

SEQ ID NO: 98 - DNA, 20 bp, Artificial Sequence
  Primer
agctcttaacacgcctggaa

SEQ ID NO: 99 - DNA, 20 bp, Artificial Sequence
  Primer
gcttaagcacgcttctgaca

SEQ ID NO: 100 - DNA, 20 bp, Artificial Sequence
  Primer
tgacatgaccagccattgat

SEQ ID NO: 101 - DNA, 20 bp, Artificial Sequence
  Primer
ccccagaaaagaacccatct

SEQ ID NO: 102 - DNA, 20 bp, Artificial Sequence
  Primer
gccgtcacctctgtcttctc

SEQ ID NO: 103 - DNA, 20 bp, Artificial Sequence
  Primer
aatttcggccattgaaagaa

SEQ ID NO: 104 - DNA, 20 bp, Artificial Sequence
  Primer
ttggatggcactgatgtgat

SEQ ID NO: 105 - DNA, 18 bp, Artificial Sequence
  Primer
gttgggcagaagagctag

SEQ ID NO: 106 - DNA, 18 bp, Artificial Sequence
  Primer
ttgccttagtccccagag

SEQ ID NO: 107 - DNA, 21 bp, Artificial Sequence
  Primer
tgcaagagacacacatatgac

SEQ ID NO: 108 - DNA, 21 bp, Artificial Sequence
  Primer
agcactctgttctcacaattg

SEQ ID NO: 109 - DNA, 26 bp, Artificial Sequence
  Primer
ggagccaagtccatgtcactgaggga

SEQ ID NO: 110 - DNA, 26 bp, Artificial Sequence
  Primer
atgcaattgctgaggaacgaaagaag

SEQ ID NO: 111 - DNA, 28 bp, Artificial Sequence
  Primer
atccacttcttcaacatactccacatcc

SEQ ID NO: 112 - DNA, 28 bp, Artificial Sequence
  Primer
gagagtgaagaatgataaggacgcaaaa

SEQ ID NO: 113 - DNA, 29 bp, Artificial Sequence
  Primer
ggtgtttttttacactcttccccccttgg

SEQ ID NO: 114 - DNA, 21 bp, Artificial Sequence
  Primer
ccaaggccacaagattctccc

SEQ ID NO: 115 - DNA, 27 bp, Artificial Sequence
  Primer
aggggagccaagtccatgtcacccagt

SEQ ID NO: 116 - DNA, 28 bp, Artificial Sequence
  Primer
gtttaggacttgtttcggtttggtggca

SEQ ID NO: 117 - DNA, 27 bp, Artificial Sequence
  Primer
tgccaccaaaccgaaacaagtcctaaa

SEQ ID NO: 118 - DNA, 26 bp, Artificial Sequence
  Primer
catccacggaccatcactttctgtta

SEQ ID NO: 119 - DNA, 28 bp, Artificial Sequence
  Primer
tgaaatgaagccaagtcctcaacatgag

SEQ ID NO: 120 - DNA, 28 bp, Artificial Sequence
  Primer
aaattacagagagacaagctgccgctgc

SEQ ID NO: 121 - protein, 23 aa, Artificial Sequence
  Synthetic Construct
XXXXXXXXXXXXXXXXXXXXXXX

SEQ ID NO: 122 - protein, 12 aa, Artificial Sequence
  Synthetic Construct
XXXXXXXXXXXX

SEQ ID NO: 123 - protein, 6 aa, Artificial Sequence
  Synthetic Construct
XXXXXX

SEQ ID NO: 124 - protein, 9 aa, Artificial Sequence
  Synthetic Construct
XXXXXXXXX

SEQ ID NO: 125 - protein, 7 aa, Artificial Sequence
  Synthetic Construct
XXXXXXX

(19) United States
(12) Patent Application Publication
(10) Pub. No.: US 2005/0204419 A1
(43) Pub. Date: Sep. 15, 2005

(54) Potato genes for resistance to late blight

(76) Inventors: John Paul Helgeson, Madison, WI (US); Sandra Austin-Phillips, Madison, WI (US); Sara Kristine Naess, Quebec (CA); Junqi Song, Madison, WI (US); Jiming Jiang, Madison, WI (US); James Mathew Bradeen, Minneapolis, MN (US); C. Rubin Buell, Olney, MD (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/755,966

(22) Filed: Jan. 12, 2004

Related U.S. Application Data
(60) Provisional application No. 60/439,376, filed on Jan. 10, 2003.

(51) Int. Cl. A01H 1/00; C12N 15/82; A01H 5/00; C07K 14/415; C12Q 1/68; C07H 21/04

(52) U.S. Cl. 800/279; 435/6; 435/69.1; 435/419; 435/468; 530/370; 536/23.6; 800/317.2

(57) ABSTRACT

Resistance genes and their encoded proteins from the wild potato, Solanum bulbocastanum, are disclosed. The genes and proteins are useful for conferring disease resistance to plants, particularly solanaceous species such as potato and tomato. In particular, the genes confer resistance to potato late blight. Compositions and methods that use the genes and proteins to enhance plant disease resistance are also disclosed, as are transgenic plants that comprise the resistance genes.

### CROSS REFERENCE TO RELATED APPLICATIONS

The present application claims priority to U.S. Provisional Application Ser. No. 60/439,376 filed Jan. 10, 2003, which is incorporated herein by reference in its entirety.

### GOVERNMENT INTERESTS

The development of the present invention was supported by USDA/ARS project funds, CRIS Project No. 3605-21000-023D and by National Science Foundation Grant Number DB19975866. The Government may have certain rights in the invention described herein.

### FIELD

The present invention relates to the field of plant physiology, genetics, and molecular biology. In particular, the invention provides novel genes and proteins useful for enhancing disease resistance in plants and methods of enhancing disease resistance in plants.

### BACKGROUND

Plant diseases cause billions of dollars in losses to farmers in the United States and elsewhere in the world every year. Generating crop plants that are naturally resistant to disease has been a goal of plant breeders for decades. Classical breeding methods have been supplemented in recent years by molecular genetic techniques, e.g., to identify a gene that encodes a protein with antifungal or antibacterial properties (often not a plant gene) and then express this gene at high levels in a plant.

Potato (Solanum tuberosum) is the world's fourth most valuable crop. In the United States, the value of the crop exceeds two billion dollars each year. Worldwide production of the cultivated potato exceeds that of all other dicot food crops (Food and Agriculture Organization, http://apps.fao.org/). Potato is also host to more than sixty pathogens of economic significance (Stevenson et al., Compendium of Potato Diseases, 2nd edn. APS Press, St. Paul (2001)) including foliar diseases, virus diseases, soil problems such as those caused by nematodes or Verticillium species, and bacterial diseases such as bacterial wilt (in the field) or Erwinia soft rot (in storage). These diseases are costly in terms of crop loss and the expenses associated with application of chemicals and environmental impact of pesticide use. Such costs could be minimized or avoided if resistant potato varieties were available. However, adequate resistance for late blight, Erwinia soft rot and many other diseases has not been incorporated into potato cultivars, partly because of the lack of a good diversity of resistance genes that breeders can use to develop resistant cultivars.

Among the most devastating diseases is late blight, a foliar and tuber disease caused by the oomycete Phytophthora infestans. Late blight was a major cause of the Irish Potato Famine, which resulted in the starvation of one million people in the mid-Nineteenth Century. The late blight fungus is also a devastating pathogen on crops other than potato. It infects tomatoes, eggplants and other solanaceous species. Other Phytophthora species are pathogenic to a wide array of agronomically important plants, including grapes, avocados and several varieties of fruit and nut trees. Despite decades of active breeding effort to control this disease, late blight still causes the loss of billions of revenue dollars for growers each year (Kamoun, Curr Opin Plant Biol 4:295-300 (2001)). Accordingly, a source of resistance to Phytophthora species that could be introduced into these species by molecular genetic techniques would also be of great value.

Possible sources of resistance to many potato pathogens exist in less adapted cultivated potato germplasm and the approximately 225 wild Solanum species. Jansky (Plant Breed Rev 19:69-155 (2000)) summarized wild and cultivated sources of resistance to nine important potato diseases, including late blight. Among wild potato species with late blight resistance is the hexaploid species Solanum demissum. Resistance from this species was first incorporated into potato via sexual crosses nearly 100 years ago (Salaman, Studies in potato breeding IV Conference Internationale de Genetique, Paris 1911. Masson, pp 573-5751911; Umaerus and Umaerus, Potato Genetics, CAB International, Wallingford, UK, pp 365-401 (1994)). A series of genes, collectively referred to as the “R” series, has been described from this species (Black and Gallegly, Am Potato J 34:273-2811957 (1957); Malcolmson and Black, Euphytica 15:199-203 (1966); Umaerus and Umaerus 1994, supra). These genes are characterized by pathogen race specificity and a hypersensitive phenotype. Although R gene-mediated resistance showed great promise initially, the late blight pathogen quickly adapted to circumvent the plant defenses (Toxopeus, Euphytica 5:221-237 (1956); Black and Gallegly, supra).

Kuhl et al. (Mol Genet Genomics, 265:977-985 (2001)) described and mapped Rpil, a late blight resistance gene from Solanum pinnatisectum. Rpil has never been deployed for potato protection and the durability potential of Rpil remains unexplored. Characterization of the Phytophthora infestans isolate used in that study led those authors to conclude that Rpil might correspond to the pathogen race specific S. demissum R9 (Kuhl et al., supra).

In 1998, somatic hybrids between cultivated potato and the wild Mexican diploid Solanum bulbocastanum (Helgeson et al., Theor Appl Genet 96:738-742 (1998), International Publication Number WO 99/05903) were generated. Somatic hybrids retained the late blight resistance of the wild species, even under intense disease pressure and without fungicide protection. Significantly, the interspecific somatic hybrids were fertile and could be backcrossed (BC) to cultivated potato. Late blight resistance segregated in BC1 populations. Full resistance could be recovered even in advanced backcross generations, indicating that the somatic hybridization method could be used to transfer resistance from wild donor species to cultivated potato, despite sexual crossing difficulties (Helgeson et al., supra). Field tests with somatic hybrid-derived materials in the Toluca Valley of Mexico, where late blight pressures are extreme, suggested that resistance derived from Solanum bulbocastanum is race non-specific (Helgeson et al., supra), unlike that associated with the previously deployed and long-since defeated R gene series from S. demissum. The successful generation of segregating somatic hybrid-derived backcross populations enabled mapping experiments, revealing a single map location on Solanum bulbocastanum chromosome 8 that imparted the late blight resistance phenotype (Naess et al., Theor Appl Genet 101:697-704 (2000), International Publication Number WO 99/05903). This region was dubbed RB (resistance region from Solanum bulbocastanum).

Clearly, there is an ongoing need to identify genes in wild potato species, such as Solanum bulbocastanum, that are responsible for disease resistance. Once isolated, these genes can then be introduced by molecular genetic techniques into domestic potato and species other than potato to confer resistance to one or more plant pathogens. This invention addresses this and other needs.

### SUMMARY OF THE INVENTION

The present invention provides, inter alia, isolated nucleic acids encoding polypeptides which, when produced in a plant, confer disease resistance in the plant, particularly solanaceous plants, and most particularly Solanum species. For instance, the present invention provides isolated nucleic acids comprising a polynucleotide at least 70% identical to a sequence as shown in SEQ ID NO: 4 or 7. The present invention also provides isolated nucleic acids comprising a polynucleotide at least 70% identical to SEQ ID NO: 1, 9 or 11. In some embodiments, the polynucleotide is at least 95% identical to SEQ ID NO: 1, 4, 7, 9, or 11. In some embodiments, the nucleic acids of the present invention encode polypeptide sequences at least 70% identical to the polypeptide sequences disclosed in SEQ ID NO: 2, 5, 8, 10, or 12. In some embodiments, the nucleic acids of the present invention encode SEQ ID NO: 2, 5, 8, 10, or 12.

The present invention also provides isolated nucleic acids comprising a polynucleotide sequence which hybridizes under stringent conditions to a sequence as shown in SEQ ID NO: 4 or 7 or the complement thereof, wherein the nucleic acid encodes a RB polypeptide. In one aspect, the polypeptide when produced in a plant confers disease resistance in the plant.

The present invention provides for polypeptides which, when produced in a plant, confer disease resistance in the plant. For instance, the present invention provides isolated polypeptides comprising amino acid sequences at least 70% identical to the amino acid sequences displayed in SEQ ID NO: 2, 5, 8, 10 or 12. In some embodiments, the isolated polypeptides are SEQ ID NO: 2, 5, 8, 10 or 13.

The present invention also provides isolated RB polypeptides encoded by an isolated nucleic acid which comprises a polynucleotide sequence that hybridizes under stringent conditions to a sequence as shown in SEQ ID NO: 4 or 7 or the complement thereof. In one aspect, the polypeptide when produced in a plant confers disease resistance in the plant.

The present invention also provides for antibodies immunologically specific for all or part, e.g., an amino-terminal portion, of a polypeptide at least 70% identical to a sequence as shown in SEQ ID NO: 2, 5, 8, 10, or 12. In one aspect of the present invention, the antibodies are immunologically specific for an amino-terminal portion of a polypeptide at least 70% identical to SEQ ID NO: 2, 5, 8, 10, or 12. Accordingly, the present invention provides isolated antibody or antibody compositions that specifically binds to a polypeptide having the amino acid sequence as shown in SEQ ID NO: 2, 5, 8, 10, or 12. In some embodiments, the antibody is monoclonal. In other embodiments, the antibody is polyclonal. In some embodiments, the antibodies of the present invention are labeled.

The present invention also provides for antibodies immunologically specific for all or part, e.g., an amino-terminal portion, of a RB polypeptide encoded by an isolated nucleic acid which hybridizes under stringent conditions to a sequence as shown in SEQ ID NO: 4 or 7 or the complement thereof.

In some aspects of the present invention, the polypeptides of the present invention confer disease resistance to a microbial pathogen. In one aspect of the present invention, the microbial pathogen is a fungus, e.g., oomycete fungus. In one aspect, the pathogen is Phytophthora infestans.

The present invention also provides recombinant expression cassettes comprising a promoter sequence operably linked to a nucleic acid of the present invention. In some embodiments, the nucleic acid comprises a polynucleotide sequence at least 70% identical to a polynucleotide sequence as shown in SEQ ID NOs: 1, 4, 7, 9, or 11. The nucleic acid can be operably linked to the promoter in a sense or antisense orientation. In even yet another embodiment, the promoter is a constitutive promoter or a tissue specific promoter.

The present invention also provides recombinant expression cassettes or vectors comprising a promoter sequence operably linked to a nucleic acid comprising a polynucleotide sequence which hybridizes under stringent conditions to a sequence as shown in SEQ ID NO: 4 or 7 or the complement thereof, wherein the nucleic acid encodes a RB polypeptide.

The invention provides antisense polynucleotides. In a preferred embodiment, the antisense polynucleotide is less than about 200 bases in length. The invention provides antisense oligonucleotides complementary to SEQ ID NOs: 4 or 7.

In some embodiments, the present invention provides host cells or progeny of host cells transformed with the recombinant expression cassettes of the present invention. In one aspect of the present invention, the host cell is a plant cell, e.g., a potato cell.

The present invention also provides transgenic plants and reproductive units of the transgenic plants. In one aspect of the present invention, transgenic plants of the present invention comprise recombinant expression cassettes comprising a promoter operably linked to a nucleic acid of the present invention. The nucleic acid can be operably linked to the promoter sequence in a sense or antisense orientation. In one embodiment of the present invention, the transgenic plant has enhanced disease resistance. In one aspect, the enhanced disease resistance is to a microbial pathogen. In another aspect, the enhanced disease resistance is to a fungus. In yet another aspect, the enhanced disease resistance is to Phytophthora infestans.

The present invention also provides methods of enhancing disease resistance in a plant. The methods comprise introducing a construct comprising a promoter operably linked to a nucleic acid of the present invention. In some embodiment, the isolated nucleic acids comprise a polynucleotide at least 70% identical to a sequence as shown in SEQ ID NO: 1, 4, 7, 9, or 11. In one aspect, the method of enhancing disease resistance enhances resistance to a microbial pathogen such as a fungus, e.g., Phytophthora infestans.

The present invention also provides kits for enhancing disease resistance in a plant. The kit comprises a construct comprising a promoter operably linked to a nucleic acid of the present invention and instructions for producing a transgenic cell using the construct.

The present invention also provides promoters. In one aspect, the invention provides isolated nucleic acid molecules for regulating expression of genes in transformed plant cells. The nucleic acid molecule comprises a segment of a gene encoding a RB gene from a plant species. In other embodiments, the nucleic acid molecule will comprise a segment of a gene encoding a RGA1, RGA3, or RGA4 gene. The segment commences at a location about 2500, preferably about 2000, bases upstream from a transcription initiation site of the gene and ends at a location about 250 bases downstream from the transcription initiation site. In one embodiment, the plant species is selected from the Solanaceae or Solanum species. In one aspect, the plant species is S. tuberosum. In one embodiment, the nucleic acid molecule controls expression of the RB, RGA1, RGA3, or RGA4 gene. In one aspect, the nucleic acid molecule is isolated from a gene having a coding sequence at least 70% identical to SEQ ID NO:7.

The present invention also provides fragments of an isolated nucleic acid molecule for regulating expression of genes in transformed plant cells. The fragments comprises a segment of a gene encoding a RB gene from a plant species. In other embodiments, the fragments will comprise a segment of a gene encoding a RGA1, RGA3, or RGA4 gene. In one aspect, the fragment comprises a segment commencing at about 2500, preferably about 2000, bases upstream from the transcription initiation site and terminating about 25 bases downstream from the transcription initiation site. In another aspect the fragment comprises a segment located between about 25 and 250 bases downstream from the transcription initiation site. The fragment is capable of increasing promoter activity of homologous or heterologous promoters.

The present invention also provides isolated nucleic acid molecules for regulating expression of genes in transformed plant cells, which comprises a segment of a gene encoding a RB gene from a plant species. In other embodiments, the nucleic acid molecule will comprise a segment of a gene encoding a RGA1, RGA3, or RGA4 gene. The segment comprises a 3′ untranslated region commencing at a stop codon for the gene's coding sequence, and ending at a location about 5900 bases downstream from the gene's transcription initiation site. In one embodiment, the plant species is selected from the Solanaceae or Solanum species. In one aspect, the plant species is S. tuberosum. In one embodiment, the nucleic acid molecule controls expression of the RB gene. In one aspect, the nucleic acid molecule is isolated from a gene having a coding sequence at least 70% identical to SEQ ID NO:7.

The present invention also provides DNA segments for effecting expression of coding sequences operably linked to the segment. The DNA segment is isolated from a gene whose coding region hybridizes under stringent conditions with a coding region defined by SEQ ID NO:7. In one aspect, the DNA further comprises an element that confers disease resistance on expression of the coding sequences. In one aspect, the segment comprises a promoter and a transcription initiation site. In another aspect, the segment comprises a polyadenylation signal. In one aspect the DNA segment is isolated from a S. Bulbocastanum RB gene.

The present invention also provides expression cassettes, cells transformed with expression cassettes, and transgenic plants comprising expression cassettes wherein the expression cassettes comprise an isolated nucleic acid molecule for regulating expression of genes in transformed plant cells, which comprises a segment of a gene encoding a RB gene from a plant species operably linked to a nucleic acid encoding a polypeptide, wherein the nucleic acid encodes a polynucleotide sequence at least 70% identical to a polynucleotide sequence as shown in SEQ ID NO:4 or SEQ ID NO:7. In other embodiments, the nucleic acid molecule will comprise a segment of a gene encoding a RGA1, RGA3, or RGA4 gene. In one aspect, the cell is a plant cell. In one aspect, the plant cell is a potato plant cell.

The invention further provides methods of detecting RB polypeptides in a sample, comprising (i) contacting the sample with an anti-RB antibody of the present invention, and (ii) determining whether a hybridization complex has been formed between the antibody and the polypeptide.

The invention further provides methods of detecting RGA1, RGA3, or RGA4 polypeptides in a sample, comprising (i) contacting the sample with an anti-RGA1, RGA3 or RGA4 antibody of the present invention, and (ii) determining whether a hybridization complex has been formed between the antibody and the polypeptide.

The invention further provides methods of detecting RB polynucleotides in a sample, comprising (i) contacting the sample with a RB polynucleotide of the present invention or a complement thereof; or contacting the sample with a polynucleotide that comprises a sequence of at least 12 nucleotides and is complementary to a contiguous sequence of a RB polynucleotide of the present invention; and (ii) determining whether a hybridization complex has been formed. In one aspect, the at least 12 nucleotide sequence will comprise a domain conserved among resistant genes and/or a LRR repeat.

The invention further provides methods of detecting RGA1, RGA3, or RGA4 polynucleotides in a sample, comprising (i) contacting the sample with a RGA1, RGA3, or RGA4 polynucleotide of the present invention or a complement thereof; or contacting the sample with a polynucleotide that comprises a sequence of at least 12 nucleotides and is complementary to a contiguous sequence of a RGA1, RGA3, or RGA4 polynucleotide of the present invention; and (ii) determining whether a hybridization complex has been formed. In one aspect, the at least 12 nucleotide sequence will comprise a domain conserved among resistant genes and/or a LRR repeat.

### BRIEF DESCRIPTION OF THE DRAWINGS

FIG. 1 is schematic map showing locations of the CAPS and SCAR markers generated for fine mapping of Solanum bulbocastanum chromosome 8.

FIG. 2 is a genetic/physical map at and near the RB region (resistance region from Solanum bulbocastanum). The lightly shaded box represents sequence information from the susceptible homology-derived BAC clone 177O13. Late blight resistance maps genetically between CAPS markers Short273C and Short274A, an area of approximately 54 kb. Genes 0-4 are found within this region (genes RGA1, RB, RGA3, and RGA4).

FIG. 3 is a schematic map showing relative position of the long range PCR primers (drawing not to scale) used to clone genes RB, RGA1, RGA3, and RGA4.

FIG. 4 is a schematic map showing the RB gene from the resistant homolog.

FIG. 5 is a schematic map showing gene RGA1 gene from the resistant homolog.

FIG. 6 is a comparison of the RB, RGA1, RGA3 and RGA4 protein sequences (SEQ ID NO:8, 2, 10, and 12 respectively). The putative leucine zipper motif and a heptad repeat motif are underlined. Asterisks represent identical residues, colons and dots indicate similar amino acids, and dashes represent deletions. The single amino acid deletion present in the leucine zipper motif is indicated by an arrow. The missing of one complete LRR (Leuricne Rich Repeat) repeat in RB protein is indicated by a box

FIG. 7 shows the structure of the RB gene and the RB protein. 7(A) Physical structure of RB gene. Two exons are indicated by open rectangles and one intron by lines angled downward. 7(B) RB protein sequences (SEQ ID NO:8). The potential leucine zipper motif, a heptad repeat motif, three kinase motifs of the NBS domain are underlined. Conserved motifs for plant resistance genes are underlined and shown in italics. The two amino acid changes (E⁴²⁰-K, K⁶⁶²-M) caused by PCR misincorporation are indicated in bold and underlined. The start point of the 3.6-kb deletion occurred in RGA2-BAC is double underlined. The LRRs are aligned according to the consensus sequence LXXLXXLXXLXLXXRXXLXXLXX (SEQ ID NO: 121), where X represents any amino acid, L represents aliphatic residues L, I, M, V, and F, and R represents N or C. Aliphatic residues L, I, M, V, and F as well as conserved N, C, and T residues are in bold. The first L and the last two Ls are not highly conserved in different LRRs.

### DETAILED DESCRIPTION

A. General Overview

It has been discovered in accordance with the present invention that chromosome 8 of the Solanum bulbocastanum wild potato species contains a region comprising novel disease resistance genes. One or more of the genes on this chromosomal segment impart resistance, e.g., race non-specific resistance to disease-causing agents such as Phytophthora infestans.

Thus, the present invention provides for the first time a disease resistance gene (“RB gene) identified and cloned from the wild potato species Solanum bulbocastanum and the protein encoded by the RB gene (“RB protein” or “RB polypeptide”). For use in the present invention, the term RB also refers to polymorphic variants, mutants, alleles, and interspecies homologs of the disease resistance RB gene and protein.

The RB gene family includes one truncated and four complete genes in both the resistant and susceptible haplotypes. The additional genes in the resistant haplotype are the RGA1, RGA3, and RGA4 genes. The present invention also provides the RGA1, RGA3, and RGA4 genes identified and cloned from the wild potato species Solanum bulbocastanum and the proteins encoded by the RGA1, RGA3, and RGA4 genes. For use in the present invention the terms RGA1, RGA3, and RGA4 also include polymorphic variants, alleles, mutants, and interspecies homologs of the RGA1, RGA3, and RGA4 gene and protein cloned from Solanum bulbocastanum.

The present invention also provides genes and proteins from the disease susceptible allele from Solanum bulbocastanum. These are the rb, rga1, rga3, and rga4 genes and proteins. It is believed that rb, rga1 and rga3 are pseudogenes.

Preferred RB, RGA1, RGA3, and RGA4 genes and proteins of the present invention modulate disease resistance in plants. In particular, RB genes and proteins of the present invention confer disease resistance, e.g., late blight disease resistance, in plants, e.g., particularly solanaceous plants, and most particularly Solanum species.

The present invention further provides recombinant expression cassettes comprising a late blight resistance gene and transgenic plants, including their progeny, having enhanced resistance to plant pathogens and other disease-causing agents, such as the oomycete fungus. In particular, the present invention provides transgenic plants having enhanced resistance to the Phytophthora species, e.g., Phytophthora infestans.

The present invention also provides methods of enhancing disease resistance in a plant using a polynucleotide of the present invention, (e.g., RB polynucleotide) and optionally, selecting for a plant with a phenotype associated with enhanced disease resistance. In some embodiments, a plant with enhanced disease resistance will be healthier and live longer than a wild type plant when exposed to a disease-causing agent. Enhanced disease resistance can be measured according to any method known to those of skill in the art. For example, a disease symptom in a test plant can be compared to a disease symptom in a control plant following contact with a pathogen, e.g., Phytophthora infestans.

The present invention also provides expression regulatory elements, and in particular a native promoter or variant of a native promoter from Solanum bulbocastanum that can be used to express the genes and proteins of the present invention, e.g., SEQ ID NO:23. In one embodiment, the promoter is a native promoter from S. bulbocastanum that controls expression of the RB gene located on chromosome 8.

A “RB polynucleotide” of the present invention (1) comprises a nucleic acid sequence comprising a coding region of from about 50 to about 10000 nucleotides, sometimes from about 100 to about 6000 nucleotides, and preferably from about 500 to about 3000 nucleotides, which hybridizes to SEQ ID NOs: 4 or 7 or the complement thereof under stringent conditions (as defined below), and conservatively modified variants thereof; (2) has substantial identity to the polynucleotide sequences of SEQ ID NOs: 4 or 7; and (3) encodes a RB polypeptide.

A preferred RB polynucleotide comprises one or more of the following sections, (1) positions 544-571 of SEQ ID NO: 4 or 7, (2) positions 763-792 of SEQ ID NO: 4 or 7, (3) positions 862-879 of SEQ ID NO: 4 or 7, (4) positions 1192-1203, (5) positions 1216-1227 of SEQ ID NO: 4 or 7, (6) positions 1417-1425 of SEQ ID NO: 4 or 7, (7) positions 28-135 of SEQ ID NO: 4 or 7, (8) positions 1762-1827 of SEQ ID NO: 4 or 7, (9) positions 2665-2682 of SEQ ID NO: 4 or 7 and/or (10) positions 2455-2910 of SEQ ID NO: 4 or 7 and encodes a RB polypeptide or functional fragment thereof.

A “RGA1 polynucleotide” of the present invention comprises (1) a nucleic acid sequence comprising a coding region of from about 50 to about 10000 nucleotides, sometimes from about 100 to about 6000 nucleotides, and preferably from about 500 to about 3000 nucleotides, which hybridizes to SEQ ID NO: 1 or the complement thereof under stringent conditions (as defined below), and conservatively modified variants thereof; (2) has substantial identity to the polynucleotide sequences of SEQ ID NO: 1; and (3) encodes a RGA1 polypeptide.

A “RGA3 polynucleotide” of the present invention comprises (1) a nucleic acid sequence comprising a coding region of from about 50 to about 10000 nucleotides, sometimes from about 100 to about 6000 nucleotides, and preferably from about 500 to about 3000 nucleotides, which hybridizes to SEQ ID NO: 9 or the complement thereof under stringent conditions (as defined below), and conservatively modified variants thereof; (2) has substantial identity to the polynucleotide sequences of SEQ ID NO:9; and (3) encodes a RGA3 polypeptide.

A “RGA4 polynucleotide” of the present invention comprises (1) a nucleic acid sequence comprising a coding region of from about 50 to about 10000 nucleotides, sometimes from about 100 to about 6000 nucleotides, and preferably from about 500 to about 3000 nucleotides, which hybridizes to SEQ ID NO: 11 or the complement thereof under stringent conditions (as defined below), and conservatively modified variants thereof; (2) has substantial identity to the polynucleotide sequences of SEQ ID NO: 11; and (3) encodes a RGA4 polypeptide.

Preferred polynucleotides encode a polypeptide useful for conferring disease resistance in a plant, e.g., resistance to late blight (e.g., SEQ ID NOs. 4 and 7). Methods of determining whether a polypeptide is useful for conferring disease resistance in a plant are described below. Nucleic acids of the present invention can also be identified by their ability to hybridize under low stringency conditions (e.g., Tm ˜40° C.) to nucleic acid probes having the sequence of SEQ ID NO: 1, 3, 4, 5, 7, 9, 11, 13, 15, 17, 19, or 21 or the complement thereof, and fragments thereof. SEQ ID NO: 1, 3, 4, 5, 7, 9, 11, 13, 15, 17, 19, or 21 are examples of polynucleotides of the present invention. Preferred disease resistant genes of the present invention are of the non-TIR (non-Toll interleukin receptor) NBS-LRR type, a classification of plant disease resistance genes (Ballvora et al., Plant J 30:361-371, 2002).

A polypeptide of the present invention has substantial identity to the amino acid sequence of SEQ ID NOs: 2, 5, 8, 10, 12, 14, 16, 18, 20, or 22 and/or binds to antibodies raised against an immunogen comprising an amino acid sequence of SEQ ID NOs: 2, 5, 8, 10, 12, 14, 16, 18, 20, or 22. Preferred polypeptides of the present invention confer disease resistance in a plant (e.g., SEQ ID NO:5, SEQ ID NO:8), and in particular, confer resistance to Phytophthora disease causing agents, e.g., Phytophthora infestans. SEQ ID NOs: 2, 5, 8, 10, 12, 14, 16, 18, 20, and 22 are examples of polypeptides of the present invention. Polypeptides of the present invention include polymorphic variants, mutants, and interspecies homologs of SEQ ID NOs: 2, 5, 8, 10, 12, 14, 16, 18, 20, or 22. Polypeptides of the present invention also include functional equivalents or fragments of SEQ ID NOs: 2, 5, 8, 10, 12, 14, 16, 18, 20, or 22.

A preferred RB polypeptide of the present invention has substantial identity to an amino acid sequence of SEQ ID NOs: 5 or 8 and/or is encoded by a polynucleotide that hybridizes under stringent conditions to SEQ ID NO: 4 or 7 or the complement thereof. A preferred RB polypeptide or functional fragment thereof comprises one or more of the following domains or motifs: (1) kinase 1a or P-loop domain (positions 182-190 of SEQ ID NO: 5 or 8), (2) kinase 2 domain (positions 255-264 of SEQ ID NO: 5 or 8), (3) kinase 3a (positions 288-293 of SEQ ID NO: 5 or 8), (4) QLPL domain (positions 398-401 of SEQ ID NO: 5 or 8), (5) CFAY domain (positions 406-409 of SEQ ID NO: 5 or 8), (6) MHD domain (position 473-475 of SEQ ID NO: 5 or 8), (7) five-heptad leucine zipper motif (positions 10-45 of SEQ ID NO: 5 or 8), (8) four heptad repeat motif (positions 588-609 of SEQ ID NO: 5 or 8), (9) KIQLCC (position 889 to 894 of SEQ ID NO: 5 or 8), (9) one or more of the following leucine rich sequences LXXLXXLXXLXLXXRXXLXXLXX (SEQ ID NO:121), LXXLXXLXXLXL (SEQ ID NO:122), LXXLXL (SEQ ID NO:123), LXXLXXLXL (SEQ ID NO:124), LXXLXXL (SEQ ID NO: 125), preferably starting at amino position 522 of SEQ ID NO:5 or 8, where X represents any amino acid, L represents aliphatic residues L, I, M, V, and F, and R represents residues N or C, and/or (10) position 819 to 970 of SEQ ID NO:5 or 8. A preferred RB polynucleotide of the present invention encodes a preferred RB polypeptide or functional fragment thereof.

In some embodiments of the present invention, a preferred RB polypeptide comprises 21 LRR repeats, and preferably, 4 repeats of SEQ ID NO: 121, 8 repeats of SEQ ID NO:122, 2 repeats of SEQ ID NO:123, 6 repeats of SEQ ID NO: 124, and 1 repeat of SEQ ID NO: 125.

A functional fragment or functional equivalent or functional homolog of a polypeptide of the present invention is a polypeptide that is homologous to the specified polypeptide but has one or more amino acid differences from the specified polypeptide. A functional fragment or equivalent of a polypeptide retains at least some, if not all, of the activity of the specified polypeptide. For example, SEQ ID NO:5 is a functional equivalent of SEQ ID NO:8. SEQ ID NO:5 shares the same amino acid sequence as SEQ ID NO:8 except for two amino acid differences. Both SEQ ID NO:5 and SEQ ID NO:8 when produced in a plant, e.g,. a plant from the Solanaceae species, confer disease resistance to late blight. A functional fragment of the present invention can include a fragment comprising positions 819 to 970 of SEQ ID NO:5 or 8.

As used herein, the phrase “nucleic acid” or “polynucleotide sequence” refers to a single or double-stranded polymer of deoxyribonucleotide or ribonucleotide bases read from the 5′ to the 3′ end. Nucleic acids can also include modified nucleotides that permit correct read through by a polymerase and do not alter expression of a polypeptide encoded by that nucleic acid.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which can be introduced to provide codon preference in a specific host cell.

A “coding sequence” or “coding region” refers to a nucleic acid molecule having sequence information necessary to produce a gene product, when the sequence is expressed.

The term “recombinant host cell” (or simply “host cell”) refers to a cell into which a recombinant expression vector has been introduced. It should be understood that the term “host cell” is intended to refer not only to the particular subject cell but to the progeny of such a cell. Because certain modifications can occur in succeeding generations due to either mutation or environmental influences, such progeny may not, in fact, be identical to the parent cell, but are still included within the scope of the term “host cell” as used herein. Methods for introducing polynucleotide sequences into various types of host cells are well known in the art.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement other transcription control elements (e.g. enhancers) in an expression cassette.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which can be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “nucleic acid construct” or “DNA construct” is sometimes used to refer to a coding sequence or sequences operably linked to appropriate regulatory sequences and inserted into a expression cassette for transforming a cell. This term can be used interchangeably with the term “transforming DNA” or “transgene”. Such a nucleic acid construct can contain a coding sequence for a gene product of interest, along with a selectable marker gene and/or a reporter gene.

The term “selectable marker gene” refers to a gene encoding a product that, when expressed, confers a selectable phenotype such as antibiotic resistance on a transformed cell.

The term “reporter gene” refers to a gene that encodes a product which is easily detectable by standard methods, either directly or indirectly.

A “heterologous” region of a nucleic acid construct is an identifiable segment (or segments) of the nucleic acid molecule within a larger molecule that is not found in association with the larger molecule in nature. Thus, when the heterologous region encodes a plant gene, the gene will usually be flanked by DNA that does not flank the plant genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. The term “DNA construct”, is also used to refer to a heterologous region, particularly one constructed for use in transformation of a cell.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication.

A solanaceous plant is a plant from the species Solanaceae. Examples of solanaceous plants include, but are not limited to, capsicum, cymphomandra, cestrum, datura, lycium, lycopersicum, nicotiana, petunia, physalis, solandra, and solanum. Preferred solanaceous plants of the present invention are from the Solanum species. A solanaceous plant of the present invention is a member of the solanaceous family of plants that is capable of being infected by a plant pathogen, such an oomycete fungus, e.g., P. infestans.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe can include natural (i.e., A, G, C, or T) or modified bases (7-deazaguanosine, inosine, etc.). In addition, the bases in a probe can be joined by a linkage other than a phosphodiester bond, so long as it does not interfere with hybridization. Thus, for example, probes can be peptide nucleic acids in which the constituent bases are joined by peptide bonds rather than phosphodiester linkages. It will be understood by one of skill in the art that probes can bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex can later bind. By assaying for the presence or absence of the probe, one can detect the presence or absence of the select sequence or subsequence.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, bryophytes, and multicellular algae. It includes plants of a variety of ploidy levels, including aneuploid, polyploid, diploid, haploid and hemizygous. The term “transgenic plant” refers to a transgenic plant and its progeny.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A polynucleotide “exogenous to” an individual plant is a polynucleotide which is introduced into the plant, or a predecessor generation of the plant, by any means other than by a sexual cross. Examples of means by which this can be accomplished are described below, and include Agrobacterium-mediated transformation, biolistic methods, electroporation, in planta techniques, and the like.

“Increased or enhanced expression or activity of a polypeptide of the present invention,” or “increased or enhanced expression or activity of a polynucleotide encoding a polypeptide of the present invention,” refers to an augmented change in activity of the polypeptide or protein. Examples of such increased activity or expression include the following: Activity of the protein or expression of the gene encoding the protein is increased above the level of that in wild-type, non-transgenic control plants. Activity of the protein or expression of the gene encoding the protein is in an organ, tissue or cell where it is not normally detected in wild-type, non-transgenic control plants (i.e. spatial distribution of the protein or expression of the gene encoding the protein is altered). Activity of the protein or expression of the gene encoding the protein is increased when activity of the protein or expression of the gene encoding the protein is present in an organ, tissue or cell for a longer period than in a wild-type, non-transgenic controls (i.e. duration of activity of the protein or expression of the gene encoding the protein is increased).

“Decreased expression or activity of a protein or polypeptide of the present invention,” or “decreased expression or activity of a nucleic acid or polynucleotide encoding a protein of the present invention,” refers to a decrease in activity of the protein. Examples of such decreased activity or expression include the following: Activity of the protein or expression of the gene encoding the protein is decreased below the level of that in wild-type, non-transgenic control plants.

“Antibodies” as used herein includes polyclonal and monoclonal antibodies, chimeric, and single chain antibodies, as well as Fab fragments, including the products of an Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules. The present invention provides antibodies immunologically specific for part or all of the polypeptides of the present invention, e.g.,. SEQ ID NOs: 2, 5, 8, 10, 12, 14, 16, 18, 20, or 22.

An “expression cassette” refers to a nucleic acid construct, which when introduced into a host cell, results in transcription and/or translation of a RNA or polypeptide, respectively. Expression cassettes can be derived from a variety of sources depending on the host cell to be used for expression. For example, an expression cassette can contain components derived from a viral, bacterial, insect, plant, or mammalian source. In the case of both expression of transgenes and inhibition of endogenous genes (e.g., by antisense, or sense suppression) one of skill will recognize that the inserted polynucleotide sequence need not be identical and can be “substantially identical” to a sequence of the gene from which it was derived.

The term “vector” is intended to refer to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

In the case of both expression of transgenes and inhibition of endogenous genes (e.g., by antisense, or sense suppression) one of skill will recognize that the inserted polynucleotide sequence need not be identical and can be “substantially identical” to a sequence of the gene from which it was derived. As explained below, these variants are specifically covered by this term.

In the case where the inserted polynucleotide sequence is transcribed and translated to produce a functional polypeptide, one of skill will recognize that because of codon degeneracy a number of polynucleotide sequences will encode the same polypeptide. These variants are specifically covered by the term “polynucleotide sequence from” a particular gene. In addition, the term specifically includes sequences (e.g., full length sequences) substantially identical (determined as described below) with a gene sequence encoding a protein of the present invention and that encode proteins or functional fragments that retain the function of a protein of the present invention, e.g., resistance to disease causing agents such as Phytophthora infestans.

In the case of polynucleotides used to inhibit expression of an endogenous gene, the introduced sequence need not be perfectly identical to a sequence of the target endogenous gene. The introduced polynucleotide sequence will typically be at least substantially identical (as determined below) to the target endogenous sequence.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison can be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

In a particularly preferred embodiment, protein and nucleic acid sequence identities are evaluated using the Basic Local Alignment Search Tool (“BLAST”) which is well known in the art (e.g., Karlin and Altschul, 1990, Proc. Natl. Acad. Sci. USA, 87:2267-2268; Altschul et al., 1997, Nuc. Acids Res., 25:3389-3402) the disclosures of which are incorporated by reference in their entireties. In particular, five specific BLAST programs are used to perform the following task: (1) LASTP and BLAST3 compare an amino acid query sequence against a protein sequence database; (2) BLASTN compares a nucleotide query sequence against a nucleotide sequence database; (3) LASTX compares the six-frame conceptual translation products of a query nucleotide sequence (both strands) against a protein sequence database; (4) BLASTN compares a query protein sequence against a nucleotide sequence database translated in all six reading frames (both strands); and (5) BLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., 1992, Science, 256:1443-1445; Henikoff and Henikoff, 1993, Proteins, 17:49-61, the disclosures of which are incorporated by reference in their entireties). Less preferably, the PAM or PAM250 matrices can also be used (see, e.g., Schwartz and Dayhoff, 1978, eds., Matrices for Detecting Distance Relationships: Atlas of Protein Sequence and Structure, Washington: National Biomedical Research Foundation, the disclosure of which is incorporated by reference in its entirety). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (see, e.g., Karlin and Altschul, 1990), the disclosure of which is incorporated by reference in its entirety. The BLAST programs can be used with the default parameters or with modified parameters provided by the user.

“Percentage of sequence identity” is determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 98.7%, or 99%. Polypeptides that are “substantially similar” share sequences as noted above except that residue positions which are not identical can differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine. Accordingly, polynucleotides of the present invention encoding a protein of the present invention include nucleic acid sequences that have substantial identity to the nucleic acid sequences of SEQ ID NOs: 1, 3, 4, 6, 7 9, 11, 13, 15, 17, 19, or 21. Polypeptides or proteins of the present invention include amino acid sequences that have substantial identity to SEQ ID NOs: 2, 5, 8, 10, 12, 14, 16, 18, 20, and 22.

The invention also relates to nucleic acids that selectively hybridize to the exemplified sequences (including hybridizing to the exact complements of these sequences). Selective hybridization can occur under conditions of high stringency (also called “stringent hybridization conditions”), moderate stringency, or low stringency.

“Stringent hybridization conditions” are conditions under which a probe will hybridize to its target subsequence, typically in a complex mixture of nucleic acid, but not to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. Moderately stringent conditions include at least one wash (usually 2) in 0.2×SSC at a temperature of at least 50° C., usually about 55° C., for 20 minutes, or equivalent conditions.

A disease resistance response refers to a change in metabolism, biosynthetic activity or gene expression that enhances a plant's ability to suppress the replication and spread of a microbial pathogen (i.e., to resist the microbial pathogen). Examples of plant disease defense responses include, but are not limited to, production of low molecular weight compounds with antimicrobial activity (referred to as phytoalexins) and induction of expression of defense (or defense-related) genes, whose products include, for example, peroxidases, cell wall proteins, proteinase inhibitors, hydrolytic enzymes, pathogenesis-related (PR) proteins and phytoalexin biosynthetic enzymes, such as phenylalanine ammonia lyase and chalcone synthase.

Preferred proteins of the present invention, when expressed in plant, confer disease resistance in the plant. The term “disease resistance” refers to any indicia of success in the resistance of disease.

Agents that induce disease defense responses in plants (which are also referred to herein as “disease-causing agents”) include, but are not limited to, microbial pathogens such as fungi, bacteria, and viruses. The phrase “useful for conferring disease resistance” refers to the ability to initiate a disease resistance response in a plant and subsequently confer disease resistance in the plant. Transgenic plants of the present invention having enhanced disease resistance have the ability to mount a disease resistance response to disease-causing agents, in particular to oomycete fungi, such as Phytophthora infestans.

The term “disease resistance genes” or “disease resistance proteins” refers to genes or their encoded proteins whose expression or synthesis confers disease resistance.

B. Isolation of Nucleic Acids Using the Methods of the Present Invention

Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998) (“Ausubel et al.”), each of which is incorporated herein by reference in its entirety.

The isolation of sequences from the genes used in the methods of the present invention can be accomplished by a number of techniques. For instance, oligonucleotide probes based on the sequences disclosed here can be used to identify the desired gene in a cDNA or genomic DNA library from a desired plant species. To construct genomic libraries, large segments of genomic DNA are generated by random fragmentation, e.g. using restriction endonucleases, and are ligated with vector DNA to form concatemers that can be packaged into the appropriate vector.

The cDNA or genomic library can then be screened using a probe based upon the sequence of a cloned gene such as the polynucleotides disclosed here. Probes can be used to hybridize with genomic DNA or cDNA sequences to isolate homologous genes in the same or different plant species.

Alternatively, the nucleic acids of interest can be amplified from nucleic acid samples using amplification techniques. For instance, polymerase chain reaction (PCR) technology can be used to amplify the sequences of the genes directly from mRNA, from cDNA, from genomic libraries or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying genes encoding a protein of the present invention from plant tissues are generated from comparisons of the sequences provided herein. For a general overview of PCR see PCR Protocols: A Guide to Methods and Applications. Innis, M, Gelfand, D., Sninsky, J. and White, T., eds, Academic Press, San Diego (1990). For examples of primers used see examples section below.

Polynucleotides can also be synthesized by well-known techniques as described in the technical literature. See, e.g., Carruthers et al., Cold Spring Harbor Symp. Quant. Biol. 47:411-418 (1982), and Adams et al., J. Am. Chem. Soc. 105:661 (1983). Double stranded DNA fragments can then be obtained either by synthesizing the complementary strand and annealing the strands together under appropriate conditions, or by adding the complementary strand using DNA polymerase with an appropriate primer sequence.

One useful method to produce the nucleic acids of the present invention is to isolate and modify the nucleic acid sequences of the present invention. Methods of sequence-specific mutagenesis of a nucleic acid are known. In addition, Ausubel et al., supra, describes oligonucleotide-directed mutagenesis as well as directed mutagenesis of nucleic acids using PCR. Such methods are useful to insert specific codon changes in the nucleic acids of the invention.

The genes or nucleic acid sequences encoding proteins of the present invention includes genes and gene products identified and characterized by analysis using the nucleic acid sequences, including SEQ ID NOs: 1, 3, 4, 6, 7, 9, 11, 13, 15, 17, 19, or 21 and protein sequences, including SEQ ID NOs: 2, 5, 8, 10, 12, 14, 16, 18, 20, and 22. Sequences encoding proteins of the present invention include nucleic acid sequences having substantial identity to SEQ ID NOs: 1, 3, 4, 6, 7, 9, 11, 13, 15, 17, 19, or 21. Polypeptides of the present invention include polypeptides having substantial identity to SEQ ID NOs: 2, 5, 8, 10, 12, 14, 16, 18, 20, and 22.

Preferred nucleic acids of the present invention encode proteins involved in disease resistance. Plant disease resistance genes frequently share a leucine rich repeat (LRR) pattern with or without a nucleotide binding site (NBS). NBS-LRR genes may be similar to the Toll interleukin receptor (TIR) or lack significant TIR homology (non-TIR). Preferred disease resistance genes of the present invention have 21 LRR repeats and a NBS domain.

Once a nucleic acid is isolated using the method described above, standard methods can be used to determine if the nucleic acid is a preferred nucleic acid of the present invention and therefore encodes a preferred protein of the present invention, e.g., by using structural and functional assays known in the art. For example, using standard methods, the skilled practitioner can compare the sequence of a putative nucleic acid sequence thought to encode a preferred protein of the present invention to a nucleic acid sequence encoding a preferred protein of the present invention to determine if the putative nucleic acid is a preferred polynucleotide of the present invention. Using standard methods, the skilled practitioner can also perform a functional assay to determine if expression or synthesis of the putative genes or proteins confers disease resistance in a plant. For example, the skilled practitioner can use the methods of Naess et al., Theor App Genet, 101:697-701 (2000), to screen a transgenic plant containing a putative disease resistant gene of the present invention for late blight resistance. After transformation of a plant cell with a putative polynucleotide of the present invention and subsequent cultivation of the cell, the resultant transgenic plant and a control plant are sprayed to run-off with a fine mist of Phytophthora infestans sporangial suspension or are otherwise inoculated with the pathogen using methods known in the art. A blight scale, with 0 indicating a dead plant and 9 no visible infection, is used to visually rate disease severity 4-5, 7, 10-11 and 14-15 days follow exposure to Phytophthora infestans. The ratings and the ranges of percentage infections associated with the rating value are as follows: 9 equals no visible infection; 8 equals less than 10% infection; 7 equals 11-25% infection; 6 equals 26-40% infection; 5 equals 41 to 60% infection; 4 equals 61-70% infection; 3 equals 71-80% infection; 2 equals 81-90% infection; 1 equals greater than 90% infection; 0 equals 100% death. A transgenic plant successfully expressing a preferred gene of the present invention will have a higher score on the blight scale than a wild type plant. Such resistant transgenic plants contain a preferred polynucleotide of the present invention.

A transgenic plant having enhanced or increased expression of a protein identical or substantially identical to a preferred polypeptide of the present invention, e.g., SEQ ID NO:5 or SEQ ID NO:8 will typically display a phenotype associated with increased disease resistance to a disease-causing agent, e.g., Phytophthora infestans. Phenotypes associated with enhanced disease resistance to a disease-causing agents can include, for example, plants with extended photosynthetic life cycles, plants with leaves that stay green for a longer duration of time, plants with an increased yield of fruit or vegetative part (e.g. tuber), plants with larger fruit, flowers, leaves, or stems, plants with improved storageability of the tuber or other agriculturally or horticulturally significant part, and/or plants substantially lacking in disease symptoms e.g., discoloration or lesions on leaves, stems, or tubers, as compared to a wild type plant following exposure to a disease-causing agent.

C. Enhancing Expression of Polypeptides of the Present Invention

The present invention provides methods of enhancing disease resistance in a plant. In one embodiment of the invention, disease resistance is enhanced by increasing expression of a gene encoding a protein of the present invention in a plant. For example, in some embodiments, the present invention provides methods of enhancing disease resistance in a plant by increasing or enhancing expression of SEQ ID NOs: 2, 5, 8, 10, or 12 in a plant. A plant with enhanced disease resistance has phenotypic characteristics that are recognizable to the skilled practitioner, e.g., normal developmental patterns after exposure to a pathogen or reduced symptoms following exposure to a pathogen.

Using specified promoters, the skilled practitioner can direct the expression of a protein of the present invention and create plants with enhanced resistance to Phytophthora infestans. For example, in some embodiments of the present invention, a tissue specific promoter can be used to create a transgenic plant with increased resistance to Phytophthora infestans. Similarly, the skilled practitioner can choose from a variety of known promoters, whether constitutive, inducible, tissue-specific, and the like to drive expression of a polynucleotide of the present invention, thereby enhancing disease resistance in a plant, e.g., Solanum bulbocastanum genotype PT29 promoters. In a particularly preferred embodiment of the present invention, the promoter used to drive expression is shown in SEQ ID NO: 23.

Any phenotypic characteristic caused by an alteration of disease resistance in a plant, e.g., enhanced resistance, can be selected for in the present invention. For example, after introducing a polynucleotide of the present invention operably linked to a desirable promoter, e.g., constitutive, tissue specific, or inducible, in a plant and regenerating the plant by standard procedures, a skilled practitioner can use standard methods to determine if the transgenic plant is a transgenic plant of the present invention, e.g., by comparing the transgenic plant to a wild type plant after exposure to a plant pathogen and looking for phenotypes associated with an alteration of disease resistance, e.g., reduced number and/or reduced size of lesions on the affected plant part.

Enhancing or increasing expression of a gene of the present invention in a plant can modulate disease resistant processes by a variety of pathways. The particular pathway used to modulate disease resistance is not critical to the present invention.

Any number of means well known in the art can be used to increase activity of a protein of the present invention in a plant. For example, the sequences, as described herein, can be used to prepare expression cassettes that enhance or increase endogenous or exogenous gene expression. Where overexpression of a gene is desired, the desired gene from a different species can be used to decrease potential sense suppression effects. Enhanced expression of polynucleotides of the present invention, is useful, for example, to enhance disease resistance in a plant. Such techniques can be particularly useful for increasing the yield of important plant crops.

Any organ can be targeted for overexpression of a protein of the present invention such as shoot vegetative organs/structures (e.g., leaves, stems, and tubers), roots, flowers, and floral or reproductive organs/structures (e.g., bracts, sepals, petals, stamens, carpels, anthers and ovules), seed (including embryo, endosperm, and seed coat) and fruit. Vascular or provascular tissues can be targeted. Alternatively, one or several genes described in the present invention can be expressed constitutively (e.g., using the CaMV ³⁵S promoter).

One of skill will recognize that the polypeptides encoded by the genes of the invention, like other proteins, have different domains which perform different functions. Thus, the gene sequences need not be full length, so long as the desired functional domain of the protein is expressed.

The polypeptides of the present invention can be used alone or in combination with other proteins or agents to enhance disease resistance. Other agents include, for example, fungicides.

D. Inhibiting Expression of Proteins of the Present Invention

In some embodiments of the present invention, expression cassettes can be used to suppress endogenous expression of a gene of the present invention.

A number of methods can be used to inhibit gene expression in plants. For instance, antisense technology can be conveniently used. To accomplish this, a nucleic acid segment from the desired gene is cloned and operably linked to a promoter such that the antisense strand of RNA will be transcribed. The expression cassette is then transformed into plants and the antisense strand of RNA is produced. In plant cells, it has been suggested that antisense RNA inhibits gene expression by preventing the accumulation of mRNA which encodes the protein of interest, see, e.g., Sheehy et al., Proc. Natl. Acad. Sci. USA, 85:8805 8809 (1988), and Hiatt et al., U.S. Pat. No. 4,801,340.

The antisense nucleic acid sequence transformed into plants will be substantially identical to at least a portion of the gene or genes to be repressed. The sequence, however, does not have to be perfectly identical to inhibit expression. The vectors of the present invention can be designed such that the inhibitory effect applies to other proteins within a family of genes exhibiting homology or substantial homology to the target gene.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments can be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of at least about 500 nucleotides is especially preferred.

Transposon insertions or tDNA insertions can be used to inhibit expression of a gene of the present invention. Standard methods are known in the art. Catalytic RNA molecules or ribozymes can also be used to inhibit expression of the genes of the present invention. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA cleaving activity upon them, thereby increasing the activity of the constructs.

A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Haseloff et al. Nature, 334:585-591 (1988).

Another method of suppression is sense suppression. Introduction of expression cassettes in which a nucleic acid is configured in the sense orientation with respect to the promoter has been shown to be an effective means by which to block the transcription of target genes. For an example of the use of this method to modulate expression of endogenous genes see, Napoli et al., The Plant Cell 2:279-289 (1990), and U.S. Pat. Nos. 5,034,323, 5,231,020, and 5,283,184.

Generally, where inhibition of expression is desired, some transcription of the introduced sequence occurs. The effect can occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 95% to absolute identity would be most preferred. As with antisense regulation, the effect should apply to any other proteins within a similar family of genes exhibiting homology or substantial homology.

For sense suppression, the introduced sequence in the expression cassette, needing less than absolute identity, also need not be full length, relative to either the primary transcription product or fully processed mRNA. This may be preferred to avoid concurrent production of some plants that are overexpressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

One of skill in the art will recognize that using technology based on specific nucleotide sequences (e.g., antisense or sense suppression technology), families of homologous genes can be suppressed with a single sense or antisense transcript. For instance, if a sense or antisense transcript is designed to have a sequence that is conserved among a family of genes, then multiple members of a gene family can be suppressed. Conversely, if the goal is to only suppress one member of a homologous gene family, then the sense or antisense transcript should be targeted to sequences with the most variance between family members.

Another means of inhibiting gene function in a plant is by creation of dominant negative mutations. In this approach, non-functional, mutant polypeptides of the present invention, which retain the ability to interact with wild-type subunits are introduced into a plant.

Expression of a polypeptide of the present invention can also be specifically suppressed by methods such as RNA interference (RNAi). A review of this technique is found in Science, 288:1370-1372, 2000, herein incorporated by reference in its entirety for all purposes. Briefly, traditional methods of gene suppression, employing anti-sense RNA or DNA, operate by binding to the reverse sequence of a gene of interest such that binding interferes with subsequent cellular processes and therefore blocks synthesis of the corresponding protein. RNAi also operates on a post-translational level and is sequence specific, but suppresses gene expression far more efficiently. Exemplary methods for controlling or modifying gene expression are provided in WO 99/49029, WO 99/53050 and WO0/75164, the disclosures of which are hereby incorporated by reference in their entirety for all purposes. In these methods, post-transcriptional gene silencing is brought about by a sequence-specific RNA degradation process which results in the rapid degradation of transcripts of sequence-related genes. Studies have shown that double-stranded RNA can act as a mediator of sequence-specific gene silencing (see, for example, Montgomery and Fire, Trends in Genetics, 14:255-258, 1998). Gene constructs that produce transcripts with self-complementary regions are particularly efficient at gene silencing.

E. Preparation of Recombinant Vectors

Typical vectors contain transcription and translation terminators, transcription and translation initiation sequences, and promoters useful for regulation of the expression of the particular nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith, Gene 8:81 (1979); Roberts, et al., Nature, 328:731 (1987); Schneider, B., et al., Protein Expr. Purif. 6435:10 (1995); Berger, Sambrook, Ausubel (all supra). A catalogue of Bacteria and Bacteriophages useful for cloning is provided, e.g., by the ATCC, e.g., The ATCC Catalogue of Bacteria and Bacteriophage (1992) Gherna et al. (eds) published by the ATCC. Additional basic procedures for sequencing, cloning and other aspects of molecular biology and underlying theoretical considerations are also found in Watson et al. (1992) Recombinant DNA Second Edition Scientific American Books, NY.

To use isolated sequences in the above techniques, recombinant DNA vectors suitable for transformation of plant cells are prepared. Techniques for transforming a wide variety of higher plant species are well known and described in the technical and scientific literature, e.g., Weising et al. Ann. Rev. Genet. 22:421-477 (1988). A DNA sequence coding for the desired polypeptide, for example a cDNA sequence encoding a full length protein, will preferably be combined with transcriptional and translational initiation regulatory sequences which will direct the transcription of the sequence from the gene in the intended tissues of the transformed plant.

For example, for overexpression, a plant promoter fragment can be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various plant genes known to those of skill.

Alternatively, the plant promoter can direct expression of the polynucleotide of the invention in a specific tissue (tissue-specific promoters), organ (organ-specific promoters) or can be otherwise under more precise environmental control (inducible promoters). Examples of tissue-specific promoters under developmental control include promoters that initiate transcription only in certain tissues, such as fruit, seeds, flowers, pistils, or anthers. Suitable promoters include those from genes encoding storage proteins or the lipid body membrane protein, oleosin. Examples of environmental conditions that can affect transcription by inducible promoters include anaerobic conditions, elevated temperature, or the presence of light.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention will typically comprise a marker gene that confers a selectable phenotype on plant cells. For example, the marker can encode biocide resistance, particularly antibiotic resistance, such as resistance to kanamycin, G418, bleomycin, hygromycin, or herbicide resistance, such as resistance to chlorosulfuron or Basta.

Nucleic acid sequences of the present invention can be expressed recombinantly in plant cells to enhance and increase levels of endogenous plant transcription factors. A variety of different expression constructs, such as expression cassettes and vectors suitable for transformation of plant cells can be prepared. A DNA sequence coding for a polypeptide described in the present invention can be combined, for example, with cis-acting (promoter and enhancer) transcriptional regulatory sequences to direct the timing, tissue type and levels of transcription in the intended tissues of the transformed plant. Translational control elements can also be used.

The invention provides a nucleic acid operably linked to a promoter which, in some embodiments, is capable of driving the transcription of the coding sequence in plants. The promoter can be, e.g., derived from plant or viral sources. The promoter can be, e.g., constitutively active, inducible, or tissue specific. In construction of recombinant expression cassettes, vectors, transgenics, of the invention, different promoters can be chosen and employed to differentially direct gene expression, e.g., in some or all tissues of a plant or animal. Typically, desired promoters are identified by analyzing the 5′ sequences of a genomic clone corresponding to the genes described here, e.g., SEQ ID NO:23.

Constitutive Promoters

A promoter fragment can be employed which will direct expression of a nucleic acid of the present invention in all transformed cells or tissues, e.g. as those of a regenerated plant. Such promoters are referred to herein as “constitutive” promoters and are active under most environmental conditions and states of development or cell differentiation. Examples of constitutive promoters include those from viruses which infect plants, such as the cauliflower mosaic virus (CaMV) 35S transcription initiation region (see, e.g., Dagless (1997) Arch. Virol. 142:183 191); the 1′- or 2′-promoter derived from T-DNA of Agrobacterium tumefaciens (see, e.g., Mengiste (1997) supra; O'Grady (1995) Plant Mol. Biol. 29:99 108); the promoter of the tobacco mosaic virus; the promoter of Figwort mosaic virus (see, e.g., Maiti (1997) Transgenic Res. 6:143 156); actin promoters, such as the Arabidopsis actin gene promoter (see, e.g., Huang (1997) Plant Mol. Biol. 33:125 139); alcohol dehydrogenase (Adh) gene promoters (see, e.g., Millar (1996) Plant Mol. Biol. 31:897 904); ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol. 208:551-565 (1989)), Gpc2 from maize (GenBank No. U45855, Manjunath et al., Plant Mol. Biol. 33:97-112 (1997)), other transcription initiation regions from various plant genes known to those of skill. See also Holtorf (1995) “Comparison of different constitutive and inducible promoters for the overexpression of transgenes in Arabidopsis thaliana,” Plant Mol. Biol. 29:637 646.

Inducible Promoters

Alternatively, a plant promoter can direct expression of the nucleic acids described in the present invention under the influence of changing conditions, e.g., changing environmental conditions. Examples of environmental conditions that can effect transcription by inducible promoters include anaerobic conditions, elevated temperature, drought, or the presence of light. Examples of developmental conditions that can affect transcription by inducible promoters include senescence and embryogenesis. Such promoters are referred to herein as “inducible” promoters. Examples include the drought-inducible promoter of maize (Busk (1997) supra); the cold, drought, and high salt inducible promoter from potato (Kirch (1997) Plant Mol. Biol. 33:897 909). Examples of developmental conditions include cell aging, and embryogenesis. Examples of promoters include the senescence inducible promoter of Arabidopsis, SAG 12, (Gan and Amasino, Science, 270:1986-1988 (1995)) and the embryogenesis related promoters of LEC1 (Lotan et al., Cell, 93:1195-205 (1998)), LEC2 (Stone et al., Proc. Natl. Acad. of Sci., 98:11806-11811 (2001)), FUS3 (Luerssen, Plant J. 15:755-764 (1998)), AtSERK1 (Hecht et al. Plant Physiol 127:803-816 (2001)), AGL15 (Heck et al. Plant Cell 7:1271-1282 (1995)), and BBM (BABYBOOM).

Alternatively, plant promoters which are inducible upon exposure to plant hormones, such as auxins or cytokinins, can be used to express the nucleic acids of the invention. Examples include the auxin response elements E1 promoter fragment (AuxREs) in the soybean (Glycine max L.) (Liu (1997) Plant Physiol. 115:397 407); the auxin-responsive Arabidopsis GST6 promoter (also responsive to salicylic acid and hydrogen peroxide) (Chen (1996) Plant J. 10: 955 966); the auxin-inducible parC promoter from tobacco (Sakai (1996) 37:906 913); a plant biotin response element (Streit (1997) Mol. Plant Microbe Interact. 10:933 937); and, the promoter responsive to the stress hormone abscisic acid (Sheen (1996) Science 274:1900 1902). The invention can also use the cytokinin inducible promoters of ARR5 (Brandstatter and Kieber, Plant Cell, 10: 1009-1019 (1998)), ARR6 (Brandstatter and Kieber, Plant Cell, 10: 1009-1019 (1998)), ARR2 (Hwang and Sheen, Nature, 413:383-389 (2001)), the ethylene responsive promoter of ERF1 (Solano et al., Genes Dev. 12:3703-3714 (1998)), and the β-estradiol inducible promoter of XVE (Zuo et al., Plant J, 24:265-273 (2000)).

Plant promoters which are inducible upon exposure to chemicals reagents which can be applied to the plant, such as herbicides or antibiotics, are also used to express the nucleic acids of the invention. For example, the maize In2 2 promoter, activated by benzenesulfonamide herbicide safeners, can be used (De Veylder (1997) Plant Cell Physiol. 38:568 577) as well as the promoter of the glucocorticoid receptor protein fusion inducible by dexamethasone application (Aoyama, Plant J., 11:605-612 (1997)); application of different herbicide safeners induces distinct gene expression patterns, including expression in the root, hydathodes, and the shoot apical meristem. The coding sequence of the described nucleic acids can also be under the control of, e.g., a tetracycline inducible promoter, e.g., as described with transgenic tobacco plants containing the Avena sativa L. (oat) arginine decarboxylase gene (Masgrau (1997) Plant J. 11:465 473); or, a salicylic acid responsive element (Stange (1997) Plant J. 11:1315 1324).

Alternatively, inducible promoters include the tetracycline repressor/operator controlled promoter, the heat shock gene promoters, stress (e.g., wounding)-induced promoters, defense responsive gene promoters (e.g. phenylalanine ammonia lyase genes), wound induced gene promoters (e.g. hydroxyproline rich cell wall protein genes), chemically-inducible gene promoters (e.g., nitrate reductase genes, glucanase genes, chitinase genes, etc.) and dark-inducible gene promoters (e.g., asparagine synthetase gene). Pathogen inducible and wound inducible promoters include, but are not limited to the prp1 promoter (Martini et al., Mol Gen Genet 1993 236(2-3): 179-86), the Fis1 promoter (Ayliffe et al., Plant Physiol, 2002, 129,169-180, Rushton et al., Plant Cell. 2002;14(4):749-62), promoters of genes encoding lipoxygenases (e.g., Peng et al., J. Biol. Chem. 269: 3755-3761, 1994); promoters of genes encoding peroxidases (e.g., Chittoor et al., Mol. Plant-Microbe Interactions 10: 861-871, 1997); promoters of genes encoding hydroxymethylglutaryl-CoA reductase (e.g., Nelson et al., Plant Mol. Biol. 25: 401-412, 1994); promoters of genes encoding phenylalanine ammonia lyase (e.g., Lamb et al., Abstract of the general meeting of the International Program on Rice Biotechnology, Malacca, Malaysia, Sep. 15-19, 1997); promoters of genes encoding glutathione-S-transferase; promoters from pollen-specific genes, such as corn Zmg13; promoters from genes encoding chitinases (e.g., Zhu & Lamb, Mol. Gen. Genet. 226: 289-296, 1991); promoters from plant viral genes, either contained on a bacterial plasmid or on a plant viral vector (e.g., as described by Hammond-Kosack et al., Mol. Plant-Microbe Interactions 8: 181-185,1994); promoters from genes involved in the plant respiratory burst (e.g., Groom et al., Plant J. 10(3): 515-522, 1996); and promoters from plant anthocyanin pathway genes (e.g., Reddy, pp 341-352 in Rice Genetics II, supra; Reddy et al., Abstract of the general meeting of the International Program on Rice Biotechnology, Malacca, Malaysia, Sep. 15-19, 1997). Pathogen-inducible promoters are particularly useful inducible promoter for use in the present invention.

Tissue-Specific Promoters

Alternatively, the plant promoter can direct expression of the polynucleotide of the invention in a specific tissue (tissue-specific promoters). Tissue specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, e.g., roots, leaves or stems, or reproductive tissues, such as fruit, ovules, seeds, pollen, pistils, flowers, or any embryonic tissue. Reproductive tissue-specific promoters can be, e.g., ovule-specific, embryo-specific, endosperm-specific, integument-specific, seed and seed coat-specific, pollen-specific, petal-specific, sepal-specific, or some combination thereof.

Suitable seed-specific promoters can be derived from the following genes: MACI from maize (Sheridan (1996) Genetics 142:1009-1020); Cat3 from maize (GenBank No. L05934, Abler (1993) Plant Mol. Biol. 22:10131-1038); vivparous-1 from Arabidopsis (Genbank No. U93215); atmyc1 from Arabidopsis (Urao (1996) Plant Mol. Biol. 32:571-57; Conceicao (1994) Plant 5:493-505); napA and BnCysP1 from Brassica napus (GenBank No. J02798, Josefsson (1987) JBL 26:12196-1301, Wan et al., Plant J 30:1-10 (2002)); and the napin gene family from Brassica napus (Sjodahl (1995) Planta 197:264-271). Fruit specific promoters include the promoter from the CYP78A9 gene (Ito and Meyerowitz, Plant Cell, 12:1541-1550 (2000)).

Promoters include the ovule-specific BEL1 gene described in Reiser (1995) Cell 83:735-742, GenBank No. U39944. See also Ray (1994) Proc. Natl. Acad. Sci. USA 91:5761 5765. The egg and central cell specific FIE1 promoter is also a useful reproductive tissue-specific promoter.

Sepal and petal specific promoters can also used be to express nucleic acids of the present invention. For example, the Arabidopsis floral homeotic gene APETALA1 (AP1) encodes a putative transcription factor that is expressed in young flower primordia, and later becomes localized to sepals and petals (see, e.g., Gustafson Brown (1994) Cell 76:131 143; Mandel (1992) Nature 360:273 277). A related promoter, for AP2, a floral homeotic gene that is necessary for the normal development of sepals and petals in floral whorls, is also useful (see, e.g., Drews (1991) Cell 65:991 1002; Bowman (1991) Plant Cell 3:749 758). Another useful promoter is that controlling the expression of the unusual floral organs (ufo) gene of Arabidopsis, whose expression is restricted to the junction between sepal and petal primordia (Bossinger (1996) Development 122:1093 1102).

A maize pollen specific promoter has been identified in maize (Guerrero (1990) Mol. Gen. Genet. 224:161 168). Other genes specifically expressed in pollen are described, e.g., by Wakeley (1998) Plant Mol. Biol. 37:187 192; Ficker (1998) Mol. Gen. Genet. 257:132 142; Kulikauskas (1997) Plant Mol. Biol. 34:809 814; Treacy (1997) Plant Mol. Biol. 34:603 611.

Promoters specific for pistil and silique valves, inflorescence meristems, cauline leaves, and the vasculature of stem and floral pedicels include promoters from the FUL gene Mandel and Yanofsky, Plant Cell, 7:1763-1771 (1995). Promoters specific for developing carpels, placenta, septum, and ovules can also used to express nucleic acids of the present invention in a tissue-specific manner. They include promoters from the SHP1 and SHP2 genes (Flanagan et al. Plant J 10:343-353 (1996), Savidge et al., Plant Cell 721-733). Promoters specific for the anther tapetum can be derived from the TA29 gene (Goldbeg et al., Philos Trans. R. Soc. Lond. B. Biol. Sci. 350:5-17).

Other promoters include those from genes encoding embryonic storage proteins. For example, the gene encoding the 2S storage protein from Brassica napus, Dasgupta (1993) Gene 133:301-302; the 2s seed storage protein gene family from Arabidopsis; the gene encoding oleosin 20 kD from Brassica napus, GenBank No. M63985; the genes encoding oleosin A, Genbank No. U09118, and, oleosin B, Genbank No. U09119, from soybean; the gene encoding oleosin from Arabidopsis, Genbank No. Z17657; the gene encoding oleosin 18 kD from maize, GenBank No. J05212, Lee (1994) Plant Mol. Biol. 26:1981-1987; and, the gene encoding low molecular weight sulphur rich protein from soybean, Choi (1995) Mol Gen, Genet. 246:266-268, can be used. The tissue specific E8 promoter from tomato is particularly useful for directing gene expression so that a desired gene product is located in fruits. Suitable promoters can also include those from genes expressed in vascular tissue, such as the ATHB-8, AtPIN1, AtP5K1 or TED3 genes (Baima et al., Plant Physiol. 126:643-655 (2001), Galaweiler et al., Science, 282:2226-2230 (1998), Elge et al., Plant J., 26:561-571 (2001), Igarashi et al., Plant Mol. Biol., 36:917-927 (1998)).

A tomato promoter active during fruit ripening, senescence and abscission of leaves and, to a lesser extent, of flowers can be used (Blume (1997) Plant J. 12:731 746). Other exemplary promoters include the pistil specific promoter in the potato (Solanum tuberosum L.) SK2 gene, encoding a pistil specific basic endochitinase (Ficker (1997) Plant Mol. Biol. 35:425 431); the Blec4 gene from pea (Pisum sativum cv. Alaska), active in epidermal tissue of vegetative and floral shoot apices of transgenic alfalfa. This makes it a useful tool to target the expression of foreign genes to the epidermal layer of actively growing shoots.

A variety of promoters specifically active in vegetative tissues, such as leaves, stems, roots and tubers, can also be used to express the nucleic acids used in the methods of the invention. For example, promoters controlling patatin, the major storage protein of the potato tuber, can be used, e.g., Kim (1994) Plant Mol. Biol. 26:603 615; Martin (1997) Plant J. 11:53 62. The ORF13 promoter from Agrobacterium rhizogenes which exhibits high activity in roots can also be used (Hansen (1997) Mol. Gen. Genet. 254:337 343). Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28:137 144); the curculin promoter active during taro corm development (de Castro (1992) Plant Cell 4:1549 1559) and the promoter for the tobacco root specific gene TobRB7, whose expression is localized to root meristem and immature central cylinder regions (Yamamoto (1991) Plant Cell 3:371 382).

Leaf-specific promoters, such as the ribulose biphosphate carboxylase/oxygenase small subunit (“Rubisco”) promoters can be used. For example, the tomato RBCS1, RBCS2 and RBCS3A genes are expressed in leaves and light grown seedlings, only RBCS1 and RBCS2 are expressed in developing tomato fruits (Meier (1997) FEBS Lett. 415:91 95). A Rubisco promoter expressed almost exclusively in mesophyll cells in leaf blades and leaf sheaths at high levels, described by Matsuoka (1994) Plant J. 6:311 319, can be used. Another leaf-specific promoter is the light harvesting chlorophyll a/b binding protein gene promoter, see, e.g., Shiina (1997) Plant Physiol. 115:477 483; Casal (1998) Plant Physiol. 116:1533 1538. The Arabidopsis thaliana myb-related gene promoter (Atmyb5) described by Li (1996) FEBS Lett. 379:117 121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16-cell stage of embryo development and persists beyond the heart stage. A leaf promoter identified in maize by Busk (1997) Plant J. 11:1285 1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio (1996) Cell 86:423-433; and, Long (1996) Nature 379:66-69; can be used. Another useful promoter is that which controls the expression of 3 hydroxy 3 methylglutaryl coenzyme. A reductase HMG2 gene, whose expression is restricted to meristematic and floral (secretory zone of the stigma, mature pollen grains, gynoecium vascular tissue, and fertilized ovules) tissues (see, e.g., Enjuto (1995) Plant Cell. 7:517 527). Also useful are kn1 related genes from maize and other species which show meristem specific expression, see, e.g., Granger (1996) Plant Mol. Biol. 31:373 378; Kerstetter (1994) Plant Cell 6:1877 1887; Hake (1995) Philos. Trans. R. Soc. Lond. B. Biol. Sci. 350:45 51. For example, the Arabidopsis thaliana KNAT1 or KNAT2 promoters. In the shoot apex, KNAT1 transcript is localized primarily to the shoot apical meristem; the expression of KNAT1 in the shoot meristem decreases during the floral transition and is restricted to the cortex of the inflorescence stem (see, e.g., Lincoln (1994) Plant Cell 6:1859 1876).

One of skill will recognize that a tissue-specific promoter may drive expression of operably linked sequences in tissues other than the target tissue. Thus, as used herein a tissue-specific promoter is one that drives expression preferentially in the target tissue, but can also lead to some expression in other tissues as well.

In another embodiment, a nucleic acid described in the present invention can be expressed through a transposable element. This allows for constitutive, yet periodic and infrequent expression of the constitutively active polypeptide. The invention also provides for use of tissue-specific promoters derived from viruses which can include, e.g., the tobamovirus subgenomic promoter (Kumagai (1995) Proc. Natl. Acad. Sci. USA 92:1679 1683) the rice tungro bacilliform virus (RTBV), which replicates only in phloem cells in infected rice plants, with its promoter which drives strong phloem specific reporter gene expression; the cassaya vein mosaic virus (CVMV) promoter, with highest activity in vascular elements, in leaf mesophyll cells, and in root tips (Verdaguer (1996) Plant Mol. Biol. 31:1129 1139).

F. Production of Transgenic Plants

DNA constructs of the invention can be introduced into the genome of the desired plant host by a variety of conventional techniques. For example, the DNA construct can be introduced directly into the genomic DNA of the plant cell using techniques such as electroporation and microinjection of plant cell protoplasts, or the DNA constructs can be introduced directly to plant tissue using biolistic methods, such as DNA particle bombardment. Alternatively, the DNA constructs can be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Biolistic transformation techniques are described in Klein et al. Nature 327:70-73 (1987).

Agrobacterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983).

Transformed plant cells which are derived by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

The nucleic acids of the invention can be used to confer desired traits on essentially any plant. Thus, the invention has use over a broad range of plants, monocots and dicots, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea. Examples include tobacco and Arabidopsis, cereal crops such as maize, wheat, rice, soybean barley, rye, oats, sorghum, alfalfa, clover and the like, oil-producing plants such as canola, safflower, sunflower, peanut and the like, vegetable crops such as tomato tomatillo, potato, pepper, eggplant, sugar beet, carrot, cucumber, lettuce, pea and the like, horticultural plants such as aster, begonia, chrysanthemum, delphinium, zinnia, lawn and turfgrasses and the like. The disease resistant genes and proteins of the present invention are particularly useful for conferring disease resistance in solanaceous plants, and in particular, in the genus Solanum, e.g., S. tuberosum. Examples of solanaceous plants include eggplant, potato, tomato, and the like. In some embodiments, the disease resistant genes and proteins of the present invention are useful for conferring disease resistance in any plant infected by a Phytophthora species including, but not limited to, grape plants, avocado plants, and fruit and nut tree varieties.

The transgenic plants of the present invention exhibit enhanced disease resistance to disease-causing agents. In particular, the transgenic plants exhibit enhanced disease resistance to microbial pathogens, and in particular to pathogens belonging to the Phytophthora species, e.g., Phytophthora infestans.

G. Detection of the Transgenic Plants of the Present Invention

Using known procedures, one of skill can screen for plants of the invention by detecting increased or decreased levels of the claimed protein in a plant and detecting the desired phenotype. Means for detecting and quantifying mRNA or proteins are well known in the art, e.g., Northern Blots, Western Blots or activity assays.

Gene amplification and/or expression can be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Various labels can be employed, most commonly radioisotopes, particularly ³²P However, other techniques can also be employed, such as using biotin-modified nucleotides for introduction into a polynucleotide. The biotin then serves as the site for binding to avidin or antibodies, which can be labeled with a wide variety of labels, such as radionuclides, fluorescers, enzymes, or the like. Alternatively, antibodies can be employed that can recognize specific duplexes, including DNA duplexes, RNA duplexes, and DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like.

In one example, after introduction of the expression cassette into a plant, the plants are screened for the presence of the transgene and crossed to an inbred or hybrid line. Progeny plants are then screened for the presence of the transgene and self-pollinated. Progeny from the self-pollinated plants are grown. The resultant transgenic plants can be examined for any of the phenotypic characteristics associated with altered disease resistance characteristics, e.g., healthier leaves following exposure to a pathogen. For example, using the methods of the present invention, overexpression of the nucleic acids or proteins described in the present invention can enhance disease resistance. The skilled practitioner can use standard methods to determine if a plant possesses the characteristics associated with enhanced disease resistance. For example, a late blight scoring system can be used to determine if a plant has enhanced resistance to Phytophthora infestans. In a preferred embodiment, the transgenic plants have enhanced disease resistance to late blight.

H. Native Promoters from S. Bulbocastanum

The present invention provides native promoters derived from S. Bulbocastanum, For example, the present invention provides promoters derived from a S. Bulbocastanum disease resistance gene, e.g., by cloning, isolating or modifying a native promoter from a disease resistance gene In particular, the present invention provides promoters from S. Bulbocastanum capable of controlling expression of the genes of the present invention. The provided promoters can be used to initiate transcription in a plant cell.

Preferred promoters of the present invention can control expression of the RB, RGA1, RGA3, and/or RGA4 genes. Accordingly, the preferred promoters can control expression of genes comprising coding regions that have substantial identity to the coding regions of SEQ ID NOs: 1, 4, 7, 9, and/or 11, e.g., preferably at least 70%, at least 80%, at least 90%, or at least 95%, 95%, 97%, 98%, 99%, or 100% identity to the coding regions of SEQ ID NOs:, 1, 4, 7, 9, and/or 11.

A promoter sequence of the present invention can be identified, for example, by analyzing the 5′, or in some instances 3′, region of a genomic clone corresponding to the disease resistant genes described here (GenBank Accession Number AY303170) A promoter sequence of the present invention can also be identified by analyzing the 5′, or in some instances 3′ region, of a gene of the present invention, e.g., RGA1, RB, RGA3, RGA4. Sequences characteristic of promoter sequences can be used to identify the promoter. Sequences controlling eukaryotic gene expression have been extensively studied. For instance, promoter sequence elements include the TATA box consensus sequence (TATAAT), which is upstream of the transcription start site. In most instances the TATA box is required for accurate transcription initiation. In plants, further upstream from the TATA box, there is typically a promoter element with a series of adenines surrounding the trinucleotide G (or T) N G. J. Messing et al., in Genetic Engineering in Plants, pp. 221-227 (Kosage, Meredith and Hollaender, eds. (1983)). A number of methods are known to those of skill in the art for identifying and characterizing promoter regions in plant genomic DNA (see, e.g., Jordano, et al., Plant Cell, 1: 855-866 (1989); Bustos, et al., Plant Cell, 1:839-854 (1989); Green, et al., EMBO J. 7, 4035-4044 (1988); Meier, et al., Plant Cell, 3, 309-316 (1991); and Zhang, et al., Plant Physiology 110: 1069-1079 (1996)).

The present invention provides expression cassettes or vectors, host cells, or transgenic plants comprising expression cassettes or vectors comprising a S. Bulbocastanum promoter operably linked to a nucleic acid encoding a polypeptide. The promoters and nucleic acids can be operably linked using recombinant techniques, such as those described supra. The promoter can be homologous or heterologous to the nucleic acid. Preferably, expression of the nucleic acids of the present invention under the control of the promoter will increase survival of the plant in response to infection with a microbial pathogen, and in particular, in response to Phytophthora infestans. Promoter activity can be measured, for example, by measuring the difference upon contact or infection with a pathogen such as Phytophthora infestans in mRNA transcribed by genes under the control of the promoter.

One example of a promoter of the present invention is shown in SEQ ID NO:23. In a preferred embodiment, SEQ ID NO:23 can control expression of the RB, RGA1, RGA3, or RGA4 genes. Accordingly, the present invention provides nucleic acid sequences with substantial identity to the nucleic acid sequence of SEQ ID NO:23 that are capable of initiating transcription of the RB, RGA1, RGA3, or RGA4 genes. The present invention also provides recombinant expression cassettes comprising the nucleic acid sequence of SEQ ID NO:23 operably linked to a disease resistance gene of the present invention as well as transgenic plants comprising expression cassettes comprising the nucleic acid sequence of SEQ ID NO:23 operably linked to a disease resistance gene of the present invention.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes. Song et al., PNAS, 2003 100:16, 9128-9133 is hereby incorporated by reference in their entirety for all purposes.

### EXAMPLES

The following examples are offered to illustrate, but not to limit the claimed invention.

### Example 1

The following example shows the identification and isolation of the 54 kb region on chromosome 8 of Solanum bulbocastanum containing the disease resistance genes of the present invention:

The generation of somatic hybrids between a single heterozygous (RB/rb) late blight resistant genotype of Solanum bulbocastanum PI 243510 and late blight susceptible cultivated potato PI 203900 and segregating BC progeny from this somatic hybrid (Helgeson et al. 1998 Theor. Appl. Genet. 96, 738-742; Naess et al. 2001 Mol. Genet. Genomics 265 694-704; Naess et al., 2001 Theor. Appl. Genet. 101 697-704) have been reported previously. All genotypes were asexually maintained as tubers and as in vitro plantlets. Additional BC progeny were generated by crossing late blight resistant somatic hybrid-derived materials with the susceptible potato cultivars Katahdin or Atlantic or with the susceptible potato breeding line A89804-7. Protocols for phenotypic analysis of late blight resistance utilizing field and greenhouse tests at the University of Wisconsin and field tests at the International Cooperative Program for Potato Late Blight (PICTIPAPA), Metepec, Mexico have been previously reported (Helgeson et al., supra). For mapping purposes, 536 BC3 genotypes were assayed for resistance to Phytophthora infestans pathotype US8 in greenhouses at Madison. Individual genotypes were replicated an average of 3.9 times (range: 1-14). Seventy-five and 199 of these BC3 lines were subsequently field tested at PICTIPAPA in 1997 and 1999, respectively. A total of 542 BC2, 1060 BC3, and 206 BC4 genotypes were screened using molecular markers.

Bacterial Artificial Chromosome (“BAC”) Library Construction—The construction of a high-quality BAC library for Solanum bulbocastanum has been previously reported (Song et al., Genome 43:199-204 (2000)). The library, prepared by partial digestion of genomic DNA with HindIII, was augmented by the generation of a second library of 68,352 clones by partial digestion of genomic DNA with BamHI. These two libraries have average insert sizes of 155 kb and 125 kb, respectively, and together provide 14× genome coverage. A third BAC library was constructed by complete digestion of genomic DNA with BamHI, yielding a total of 8,448 clones in BAC vectors pBeloBACII (Shizuya et al., Proc Natl Acad Sci USA 39:8794-8797 (1992); 2,304 clones) and pCLD04541 (Bent et al. Science 265:1856-18601994; 6,144 clones). All three BAC libraries were prepared from the same single RB/rb Solanum bulbocastanum PI 243510 genotype used in the initial somatic fusion via previously reported methodologies (Song et al., supra), except where noted. The BAC library was arrayed onto nylon filters (Hybond N+, Amersham Biosciences, Piscataway, N.J.) via manual or robotic (QBot, Genetix Limited, Hampshire, UK) methods.

BAC Library Screening and Contig Construction via BAC Walking—BAC membranes were screened via Southern hybridization. Probes for Southern hybridizations included tomato-derived genomic clones TG261 and TG495, tomato-derived cDNA clones CT64 and CT88, and RAPD marker G02, markers previously shown to be associated with RB-mediated late blight resistance (Naess et al., supra).

Additional probes were developed from the end sequences of select BAC clones associated with the region via PCR amplification of BAC template DNA. PCR primers were designed from BAC end sequences using Primer3 software (http://www-genome.wi.mit.edu/cgi-bin/primer/primer3_www.cgi) and synthesized by Integrated DNA Technologies (Coralville, IA). Reactions were carried out in a total volume of 50 μl containing 1× reaction buffer containing 1.5 mM MgCl₂, supplied by the manufacturer (Applied Biosystems, Foster City, Calif.), 0.2 mM each dNTP, 1 U AmpliTaq DNA Polymerase (Applied Biosystems), 50 ng template DNA (BAC clone or genomic Solanum bulbocastanum), and 50 pmol each PCR primer. Thermocycler (Applied Biosystems) conditions were 1 min at 94 C. followed by 35 cycles of 30 sec at 94 C., 45 sec at 52 C., and 1 min at 72 C. Five microliter aliquots of each completed PCR were visually characterized for amplification efficiency and correct product size following electrophoresis through a 0.8% TBE agarose gel, ethidium bromide staining, and uv visualization. Provided reaction efficiency and accuracy were acceptable, the remainder of each reaction was purified using a MicroSpin S-200 HR column (Amersham Biosciences) per manufacturer's instructions. Five microliters of purified PCR-generated or 100 ng of tomato- and RAPD-derived fragments were used in a P-32 labeling reaction using the DECAprime II Random Priming DNA Labeling Kit (Ambion, Inc., Austin, Tex.). Unless previously known to be low-copy number in nature, each probe was initially tested on a single BAC membrane containing 1,536 clones. Probes confirmed to be low copy number were subsequently used to screen the entire collection of BAC clones using formamide-based hybridization (Sambrook and Russell 2001). Probes were used singly or were multiplexed in pairs or triples. The following table, Table 1, lists all probes used to screen the BAC libraries and, where applicable, primer sequence information for the generation of the probe fragment and amplicon size.

TABLE 1

Probes used for BAC contig initiation and
extension^{a}

| | Sub- | | |
|---|---|---|---|
| | contig | | |
| | re- | Probe | Primer sequences (ampli- |
| Probe | gion^{b} | origin | con size) or reference |
| G02 | G02 | RAPD | GGCACTGAGG^{c} |
| | | | (SEQ ID NO: 36) |
| 5O15F | G02 | BAC end | CCCTTCTTATTCTTTAAGCAAACTT; |
| | | | (SEQ ID NO: 37) |
| | | | ATTTTTCCCCTCCCAGCTT |
| | | | (107 bp) |
| | | | (SEQ ID NO: 380) |
| 5015R | G02 | BAC end | ATTTTGCTCGAATCGCTCAT; |
| | | | (SEQ ID NO: 39) |
| | | | TCCCAATTGGATCACTTGTCT |
| | | | (245 bp) |
| | | | (SEQ ID NO: 40) |
| 12B7F | G02 | BAC end | TGTTTGTGCATTGAAGATTGG; |
| | | | (SEQ ID NO: 41) |
| | | | TGGTAAGAAGGGCATTCCATA |
| | | | (162 bp) |
| | | | (SEQ ID NO: 42) |
| 32A7a | G02 | BAG end | GGAAATACTAGAGGGGAGGGAGT; |
| | | | (SEQ ID NO: 43) |
| | | | TGAATAAGCAGTTCGGTTTGAA |
| | | | (493 bp) |
| | | | (SEQ ID NO: 44) |
| 32A7b | G02 | BAC end | TCTCTTGGGATCACGATTCA; |
| | | | (SEQ ID NO: 45) |
| | | | TTTAATTTCGGCGGATGAAC |
| | | | (367 bp) |
| | | | (SEQ ID NO: 46) |
| 51H24F | G02 | BAC end | TTTGGAGGATAGCAATACTTGGA; |
| | | | (SEQ ID NO: 47) |
| | | | AGCAACTGGTGAGAAAATGTCTT |
| | | | (189 bp) |
| | | | (SEQ ID NO: 48) |
| 219D10F | G02 | BAC end | CGGTTCAGCTGACCTTTCAT; |
| | | | (SEQ ID NO: 49) |
| | | | ACCTGCGAGTGGATCAAAAC |
| | | | (103 bp) |
| | | | (SEQ ID NO: 50) |
| CT88 | CT88/ | Tomato | Naess, et al. (2000) |
| | TG495 | cDNA | |
| TG495 | CT88/ | Tomato | Naess, et al. (2000) |
| | TG495 | genomic | |
| 7O17R | CT88/ | BAC end | TCACAATGCTAATATGTGGTTTGA; |
| | TG495 | | (SEQ ID NO: 51) |
| | | | AGTTGTTTGTGGCTGCCATT |
| | | | (297 bp) |
| | | | (SEQ ID NO: 52) |
| 12F6F | CT88/ | BAC end | AGGTGTCCAAGTGAAAAGTCG; |
| | TG495 | | (SEQ ID NO: 53) |
| | | | ATCAAGCACCTCCCCAAAC |
| | | | (170 bp) |
| | | | (SEQ ID NO: 54) |
| 49N10F | CT88/ | BAC end | AACTAGCCCGCGATCAACTA; |
| | TG495 | | (SEQ ID NO: 55) |
| | | | AAACCGACACAGATGCAACA |
| | | | (365 bp) |
| | | | (SEQ ID NO: 56) |
| 52M2F | CT88/ | BAC end | CCCTCTGTTCCGTGACAAAT; |
| | TG495 | | (SEQ ID NO: 57) |
| | | | CACAGAAGGGGGTTGATCTC |
| | | | (359 bp) |
| | | | (SEQ ID NO: 58) |
| 52M2R | GT88/ | BAC end | TGAGTTCCACAGTCTGTACATAACAA; |
| | TG495 | | (SEQ ID NO: 59) |
| | | | TTTCTTCCTCTCCCTTCTCCTT |
| | | | (350 bp) |
| | | | (SEQ ID NO: 60) |
| 64K8R | CT88/ | BAC end | AACAAGATGAGCCTGGTGTG; |
| | TG495 | | (SEQ ID NO: 61) |
| | | | ATCACATCCCAGAGGCAAAA |
| | | | (349 bp) |
| | | | (SEQ ID NO: 62) |
| 80G6F | CT88/ | BAC end | ATCAATCCATCATGTGAGCA; |
| | TG495 | | (SEQ ID NO: 63) |
| | | | TCAGAAAATAAGCACGTTTGACA |
| | | | (122 bp) |
| | | | (SEQ ID NO: 64) |
| 80G6R | CT88/ | BAC end | CTTGAGAAGGCAACGACAGA; |
| | TG495 | | (SEQ ID NO: 65) |
| | | | GAAGGCGGGTAAACAGACAG |
| | | | (231 bp) |
| | | | (SEQ ID NO: 66) |
| 117J16F | CT88/ | BAC end | CAATCGCTCCTTCCAACTTC; |
| | TG495 | | (SEQ ID NO: 67) |
| | | | TGAGCAGCATTCGAAGAAAA |
| | | | (361 bp) |
| | | | (SEQ ID NO: 68) |
| 122E4R | CT88/ | BAC end | AGGAATCTCCTCAAGTTCTACACA; |
| | TG495 | | (SEQ ID NO: 69) |
| | | | GATACGGGTGCCAGGATTC |
| | | | (103 bp) |
| | | | (SEQ ID NO: 70) |
| 157M5F | CT88/ | BAC end | TTCAACCAGCAAGTTCAAGC; |
| | TG495 | | (SEQ ID NO: 71) |
| | | | TTATTGTCCATGTCGCTCCA |
| | | | (357 bp) |
| | | | (SEQ ID NO: 72) |
| 201A16F | CT88/ | BAC end | GTTCCCATGCCTAAACCAGA; |
| | TG495 | | (SEQ ID NO: 73) |
| | | | ATCGCCCGCTCAACTTAATA |
| | | | (115 bp) |
| | | | (SEQ ID NO: 74) |
| 201A16R | CT88/ | BAC end | TGAGGTATTGCTGTGGGTTG; |
| | TG495 | | (SEQ ID NO: 75) |
| | | | TGAATTCAGCCCAGAAGTGAA |
| | | | (103 bp) |
| | | | (SEQ ID NO: 76) |
| CT64 | CT64/ | Tomato | Naess, et al. (2000) |
| | TG261 | cDNA | |
| TG261 | CT64/ | Tomato | Naess, et al. (2000) |
| | TG261 | genomic | |
| 61A13R | CT64/ | BAC end | ATTCAATCGCCTGTCCAAAC; |
| | TG261 | | (SEQ ID NO: 77) |
| | | | CATGCTCTCGTTGGATGAA |
| | | | (333 bp) |
| | | | (SEQ ID NO: 78) |
| 103H7F | CT64/ | BAC end | CCCTCGACATGAACCAGAAG; |
| | TG261 | | (SEQ ID NO: 79) |
| | | | TGTCCATGTAGGCCAAGACC |
| | | | (352 bp) |
| | | | (SEQ ID NO: 80) |
| 120C9F | CT64/ | BAC end | ACAGGCCAGGGTTCAAAATA; |
| | TG261 | | (SEQ ID NO: 81) |
| | | | GCAATGGACAGACTTGATGC |
| | | | (378 bp) |
| | | | (SEQ ID NO: 82) |

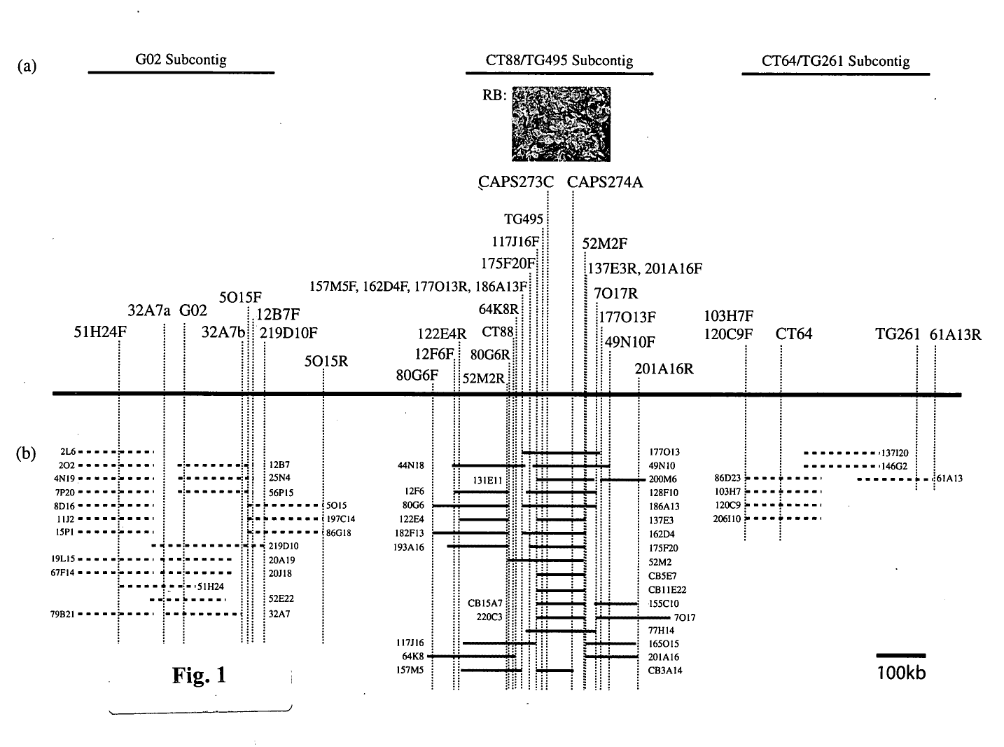

^{a}Chromosome location of each probe is indicated in FIG. 1
^{b}Subcontig regions are as defined in FIG. 1
^{c}Operon G-02, Westburg BV, Leusden, The Netherlands; Naess et al. (2000)

The identity of single colony isolates for each positive BAC clone was reconfirmed by Southern hybridization using the same probe or probes used to detect them initially. In instances in which a group of clones were identified in multiplexed Southern hybridizations, the reconfirmation step was repeated for each probe separately. For positive BAC clones, an average of 552 bp of sequence information was generated from each end using previously reported methods (Zhao et al., Genome Res 11: 1736-1745 (2001)).

BAC walking involved the reiterative screening of the BAC library using probes derived from the ends of previously identified BAC clones. Each probe identified multiple BAC clones. Clones were arranged into subcontig groupings via cross hybridization of individual BAC ends; BAC ends present at the termini of the subcontig groupings hybridized only to the BAC clones from which they were derived. PCR generated probes from terminal BAC clones were subsequently used to screen the BAC libraries, as described above.

Insert size was estimated for selected BACs using the methods of Song et al. (2000). BAC clone 177O13 was partially sequenced using a shotgun approach as reported previously (Yuan et al., Mol Genet Genomics 267:713-720 (2002)).

Reiterative Fine Genetic Mapping and Determination of Homolog Origin—Cleaved Amplified Polymorphic Sequences (CAPS) and Sequence Characterized Amplified Region (SCAR) markers were developed from partial BAC sequences and BAC end sequences. PCR primers were selected manually or using Primer3 software to maximize amplicon size. PCR products were generated in a total volume of 25 μl containing 1× reaction buffer (Applied Biosystems), 2 mM MgCl₂, 0.2 mM each dNTP, 1 U AmpliTaq DNA Polymerase (Applied Biosystems), approximately 15 ng genomic DNA, and 5 pmol each PCR primer (Integrated DNA Technologies). Standard thermocycler (Applied Biosystems) conditions were 94° C. for 2 minutes followed by 30 cycles of 94° C. (1 min), 55° C. (30 sec), 72° C. (90 sec). When necessary, annealing temperatures were adjusted to match the predicted melting temperature of the primer pair.

Initially, primers were used to generate fragments from the RB/rb Solanum bulbocastanum PI 243510 genotype used in somatic hybridization and BAC library construction, late blight susceptible cultivated potato cultivar Katahdin or line R4, and a late blight resistant and late blight susceptible potato+Solanum bulbocastanum somatic hybrid-derived BC1 line. Polymorphic fragments were subsequently mapped to Solanum bulbocastanum chromosome 8 using a set of 8 resistance and 8 susceptible BC1 lines. For CAPS markers, entire PCRs were digested by the addition of 2 ul of enzyme mixture composed of 2 U of CfoI, DraI, RsaI, TaqI (all from Promega Corporation, Madison, Wis.), or TfiI (New England Biolabs, Inc., Beverly, Mass.) and 1× restriction enzyme buffer (supplied by manufacturer), followed by incubation for 2 hours at 37 C. (CfoI, DraI, and RsaI) or 65 C. (TaqI and TfiI). Digested products were separated by electrophoresis through a 0.6% agarose+1.0% Synergel (DiversifieD Biotech, Boston, Mass.) TBE gel. Polymorphisms were visualized using ethidium bromide stain and ultraviolet light irradiation. Polymorphic products with easily distinguished patterns were subsequently used to screen advanced BC populations.

DNA was extracted from single cotyledons of BC individuals using the microprep method of McGrath et al. (Theor Appl Genet 88:917-924 (1994)). All individuals were tested for the presence of markers flanking resistance [G02 and 103H7R or G02 and P09 (Naess et al. 2000)]; recombinant individuals were tested with additional markers (CT88, 137E3R, 177O13R, 186A3F) to further pinpoint the recombination site. The following table, Table 2, summarizes CAPS and SCAR markers generated for fine mapping, including PCR primers, product size, and, where appropriate, polymorphic enzyme site. Map locations are indicated in FIG. 1. BAC clones associated with an approximately 55 kb region shown genetically to be responsible for the resistance phenotype were screened using genetically mapped CAPS or SCAR markers to determine homolog (RB vs. rb) origins.

TABLE 2

PCR-based markers developed for fine genetic mapping of the RB region^{a}

| | | |
|---|---|---|
| Marker | Marker | Fragment sizes (bp)^{c} |

| | | | | | | |
|---|---|---|---|---|---|---|
| Marker | type | behavior^{b} | Primers | Amplicon | RB homolog | rb homolog |

| | | | | | | |
|---|---|---|---|---|---|---|
| 52M2F | CAPS | dominant | GAGGCAAACCCTCTGTTCCGT | 596 | 596 | 596 |
| | | | (SEQ ID NO: 83) | | | |
| | | | GCTCCAAGTGGAGGAAATGCC | | | |
| | | | (SEQ ID NO: 84) | | | |
| 64K8R | CAPS | dominant | AACAAGATGAGCCTGGTGTG | 349 | 349 | 349 |
| | | | (SEQ ID NO: 85) | | | |
| | | | ATCACATCCCAGAGGCAAAA | | | |
| | | | (SEQ ID NO: 86) | | | |
| 103H7F | CAPS | dominant | GCTTAGTGCCCTTAAGCG | 549 | 299 + (37) + 213 | 299 + (37) + 213 |
| | | | (SEQ ID NO: 87) | | | |
| | | | CTGACTAACCGGATGGCC | | | |
| | | | (SEQ ID NO: 88) | | | |
| 137E3R | SCAR | codominant | AAAATTGTCCTCCTCTAATTTTCTTT | 750^{d}/307 | 750^{d} | 307 |
| | | | (SEQ ID NO: 89) | | | |
| | | | TGATATGAAAAGAAAGTGGTTGC | | | |
| | | | (SEQ ID NO: 90) | | | |
| 162D4F | CAPS | codominant | CGTGAAGTGAAATGCTCAACA | 569 | 569^{d} | (17) + 372 + 180 |
| | | | (SEQ ID NO: 91) | | | |
| | | | GCAAACTTTGGAAGGATTCG | | | |
| | | | (SEQ ID NO: 92) | | | |
| 175F20F | SCAR | dominant | CCTGAGCCTCGGTGAGAGTA | 353 | 353 | 353 |
| | | | (SEQ ID NO: 93) | | | |
| | | | ACCCAAAACTCCCAACCTCT | | | |
| | | | (SEQ ID NO: 94) | | | |
| 177O13F | SCAR | dominant | CTGGTTTGACAATGCTGGTG | 598 | 598 | 598 |
| | | | (SEQ ID NO: 95) | | | |
| | | | GACACTCAAGGCTGCCATTT | | | |
| | | | (SEQ ID NO: 96) | | | |
| 177O13R | CAPS | codominant | TCTGCAGAAAACCATCTCAGG | 400^{d}/380 | 400^{d} | 380 |
| | | | (SEQ ID NO: 97) | | | |
| | | | AGCTCTTAACACGCCTGGAA | | | |
| | | | (SEQ ID NO: 98) | | | |
| 186A13F | CAPS | codominant | GCTTAAGCACGCTTCTGACA | 331 | 331 | 193 + 138 |
| | | | (SEQ ID NO: 99) | | | |
| | | | TGACATGACCAGCCATTGAT | | | |
| | | | (SEQ ID NO: 100) | | | |
| CAPS273C | CAPS | dominant/ | CCCCAGAAAAGAACCCATCT | 1008 | TaqI: 1008 | TaqI: 1008 |
| | | | (SEQ ID NO: 101) | | | |
| | | codominant^{e} | GCCGTCACCTCTGTCTTCTC | | DraI: 1008 | DraI: |
| | | | (SEQ ID NO: 102) | | | 573 + 435 |
| CAPS274A | CAPS | codominant | AATTTCGGCCATTGAAAGAA | 1012 | 1012 | 162 + 850 |
| | | | (SEQ ID NO: 103) | | | |
| | | | TTGGATGGCACTGATGTGAT | | | |
| | | | (SEQ ID NO: 104) | | | |
| CT88 | CAPS | dominant | GTTGGGCAGAAGAGCTAG | 591 | (70) + 136 + 385 | (70) + 136 + 385 |
| | | | (SEQ ID NO: 105) | | | |
| | | | TTGCCTTAGTCCCCAGAG | | | |
| | | | (SEQ ID NO: 106) | | | |
| TG495 | CAPS | codominant | TGCAAGAGACACACATATGAC | 766/809^{f} | 205 + 488 + (73) | 244 + 488 + (77) |
| | | | (SEQ ID NO: 107) | | | |
| | | | AGCACTCTGTTCTCACAATTG | | | |
| | | | (SEQ ID NO: 108) | | | |

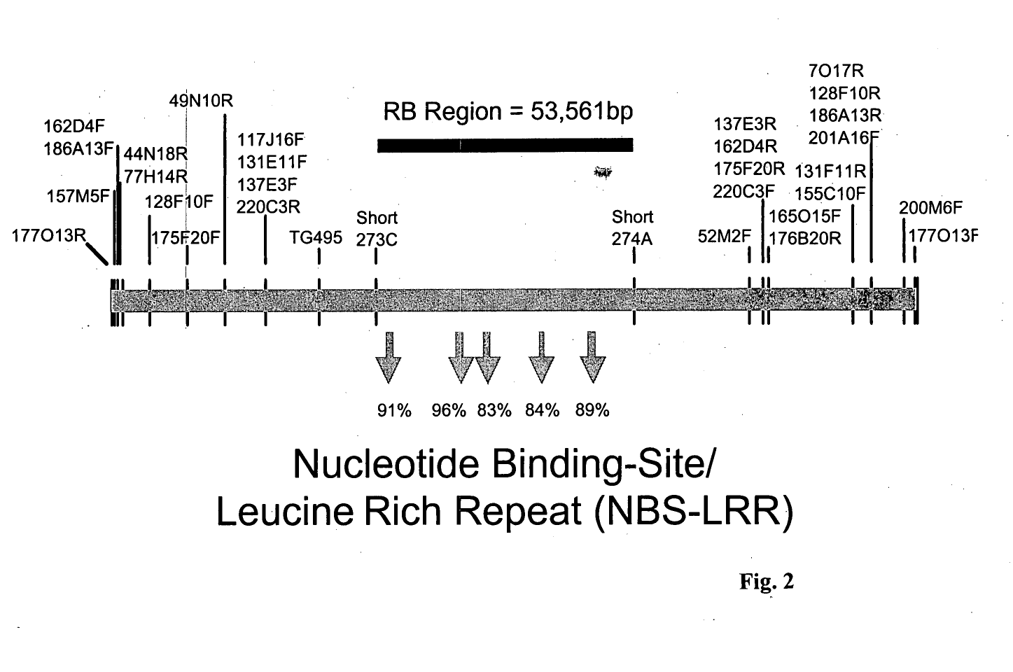

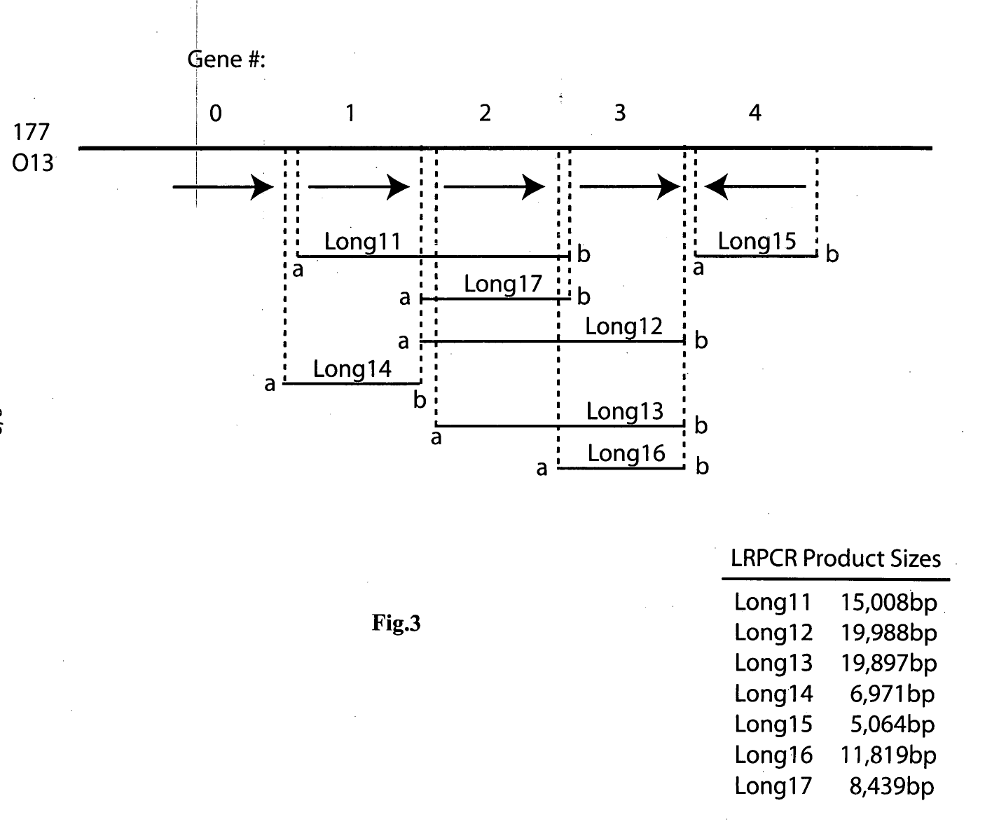

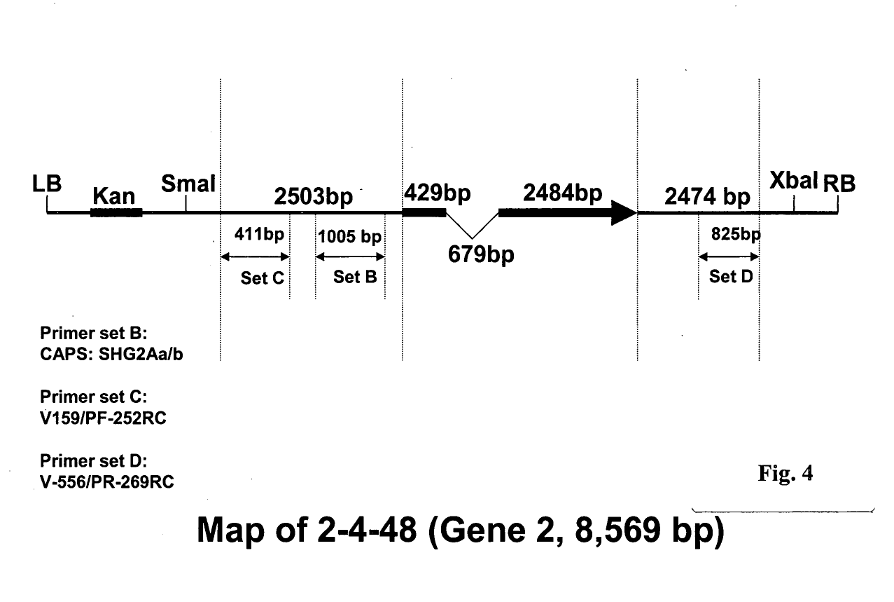

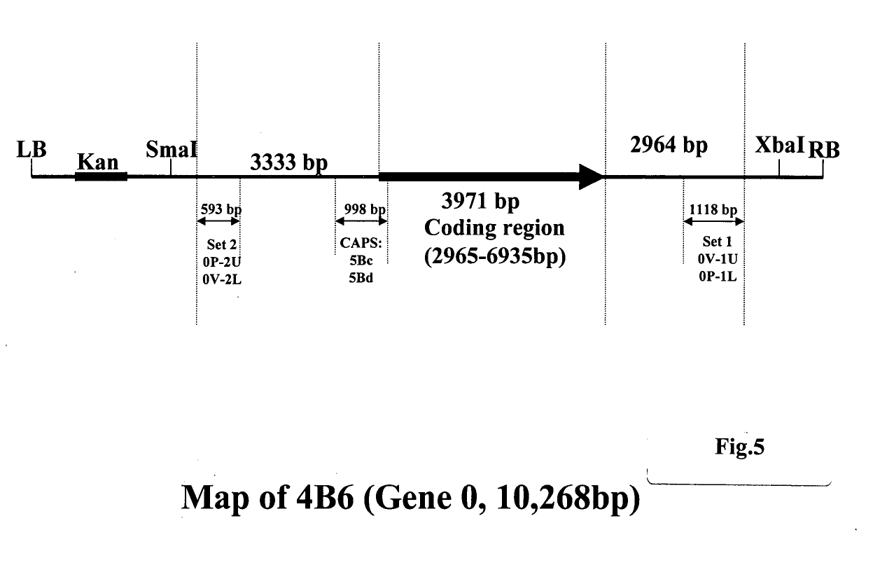

^{a}Chromosome location of each marker is indicated in FIG. 2
^{b}Dominant markers distinguish between cultivated potato and S. bulbocastanum but do not distinguish between RB and rb homologs; codominant markers distinguish between S. bulbocastanum RB and rb homologs
^{c}Fragment sizes in parentheses are not always visible on agarose gels
^{d}Size is approximated from agarose gel
^{e}TaqI reveals a dominant polymorphic site present in cultivated potato but lacking in the S. bulbocastanum RB and rb homologs; DraI reveals a codominant polymorphic site present in the S. bulbocastanum rb homolog but absent in both cultivated potato and the S. bulbocastanum RB homolog
^{f}RB homolog product for TG495 CAPS is 766 bp in length; size polymorphism is readily distinguished in FokI digest

Contig Development and Extension—Previous mapping of the RB phenotype revealed a single chromosome region on the Solanum bulbocastanum chromosome 8 responsible for late blight resistance (Naess et al., supra). The region explains 62.1% of the observed phenotypic variation and no other genome location was significantly correlated with resistance. Initial linkage mapping of 114 BC2 individuals placed resistance between RAPD G02 and RFLP CT64 (Naess et al. supra; unpublished results of S. K. Naess, S. M. Wielgus, and J. P. Helgeson). No recombination was observed between resistance and RFLP markers CT88 and TG495 (Naess et al., supra). In the present study, markers G02, CT88, TG495, CT64, and TG261 were used as probes to initiate physical map and BAC contig development for the RB region.

Several BAC clones were identified from the HindIII and BamHI partial digestion libraries with each probe. Following single colony isolation and confirmation of identity, BAC clones were end sequenced and the sequence information was used to design new probes from each end of each BAC via PCR amplification. BAC end probes were subsequently hybridized to all overlapping clones (i.e. those BACs initially identified using a single, common probe) and the observed patterns of cross-hybridization were used to orient individual clones and order the entire subcontig grouping. Throughout the entire RB region, approximately one in six BAC end probes was moderately to highly repetitive. Prior to screening the library en masse, each probe was tested using a single BAC filter of 1,536 clones. Low copy number probes from the termini of BAC subcontig groupings (or internal to the grouping for cases in which terminal probes were repetitive) were used singly, in pairs, or in triples to screen the BAC library, extending subcontig clusters by BAC walking. Following isolation of individual colonies, all newly identified BAC clones were hybridized with each terminal BAC probe individually, both confirming clone association with RB and determining homology between each BAC and a specific probe.

Fine Mapping using PCR-Based Markers of Known Physical Proximity to RB—The generation of BAC end sequence data for RB-related BACs and partial sequence from BAC clone 177O13 provided opportunity to develop additional, PCR-based (CAPS and SCAR) markers for fine genetic mapping of the RB phenotype. The development and application of PCR-based markers greatly enhanced genetic resolution within the RB region substantially reducing the physical size for which contig development was necessary.

CAPS or SCAR markers for genetic fine mapping were developed from BAC sequences that were known a priori to be physically closer to late blight resistance. Using a reiterative process of concomitant subcontig grouping extension and fine genetic mapping, it was possible to confine the genetic factors responsible for resistance to a region defined by RFLP TG495 and BAC end 52M2F. BAC clone 177O13, which entirely encompasses the TG495/52M2F region, was subsequently selected for sequencing. CAPS 273C and CAPS 274A were ultimately developed from 177O13 partial sequence. Based on subcontig models that incorporate calculated BAC sizes, the CAPS 273C/CAPS 274A interval was estimated to be approximately 55 kb in length. Genetic mapping confirmed that late blight resistance lies entirely within this region. By “redefining” the RB region using this reiterative fine mapping approach, it was possible to focus physical mapping efforts exclusively on the CAPS 273C/CAPS 274A region.

The use of PCR-based markers allowed non-destructive assay of mapping individuals at the cotyledon stage. Individuals non-recombinant between markers flanking late blight resistance provided no added map resolution and were discarded, long before phenotypic analysis of late blight resistance ensued. This allowed rapid and cost-effective screening of greatly expanded mapping populations.

Homolog Determination—BAC walking and reiterative fine mapping allowed efficient development of a BAC contig spanning the genetically-defined RB region. Because the single Solanum bulbocastanum genotype used in BAC library construction was heterozygous for late blight resistance (RB/rb), it was necessary to determine homolog origin for all BACs in the immediate RB region, essentially separating BACs into an RB and an rb contig. Codominant CAPS and SCAR markers, previously used for fine genetic mapping, were invaluable for homolog determination. Assuming the BAC libraries represent an unbiased sampling of the Solanum bulbocastanum genome, random probability suggests that 50% of BAC clones should originate from the RB homolog and 50% from the rb homolog. However, of 11 BAC clones encompassing the 55 kb, genetically-defined RB region isolated from the HindIII and BamHI partial digestion libraries, all 11, including the partially sequenced 177O13, originated from the susceptible rb homolog. These BAC clones are not expected to carry alleles that impart late blight resistance. In contrast, BACs of known homolog origin flanking the genetically-defined RB region showed segregation of homolog origin with three of four and four of five BAC clones originating from the resistant RB homolog. BAC libraries developed from partial digestion of genomic DNA with HindIII and BamHI were exhaustively screened for RB region clones.

Subsequently, a third BAC library was constructed from the same heterozygous Solanum bulbocastanum genotype. This library was prepared by complete digestion of genomic DNA with BamHI, guaranteeing minimally-sized fragments incorporating the genetically-defined RB region. Among the 4 BAC clones recovered from the newly-prepared library using RFLP probe TG495 was a single clone, derived from the resistant RB region, of approximately 80 kb. GenBank Accession no. AY303170 (CB3A14) provides the region from chromosome 8 comprising the disease resistant genes. GenBank Accession No. AY303171 (177O13) provides the resistant homolog.

### Example 2

The following example shows the primers used to isolate the disease resistance genes from the resistant homolog:
DNA was extracted from leaves of greenhouse-grown Solanum bulbocastanum genotype PT29 plants using the method of Fulton et al., Plant mol biol report 13:207-209 (1995), purified on a cesium chloride gradient, and quantified via fluorometry.

Long range PCR products were generated using the following reaction conditions: 0.75 ul (=96 ng) template DNA, 5.0 ul 10×PCR buffer, provided by Manufacturer (Panvera, Madison, Wis.), 8.0 ul dNTP mix (=400 uM each dNTP, final concentration; provided by Panvera), 33.75 ul ddH2O, 0.5 ul Taq Polymerase (Takara LA Taq Polymerase, Panvera; =2.5 U), 1.0 ul Primer “a” (=10 pmol). 1.0 ul Primer “b” (=10 pmol). The Thermocycler (Applied Biosystems 9700) conditions were as follows: 94° C. (1 min), 14 cycles of: 94° C. (10 sec)+60° C. (10 min)+72° C. (15 min), 16 cycles of: 94° C. (10 sec)+60° C. (10 min)+72° C. (15 min+15 sec/cycle), 72° C. 10 min, and 4° C. (hold indefinitely).

Long range PCRs were run on 1.0% TBE or TAE agarose and visualized using ethidium bromide staining and uv light. PCRs were checked for reaction efficiency and fragment size.

The primers used for amplification of the promoter, terminator and coding regions for RGA1 and RB genes were:

| | |
|---|---|
| LONG 11a | |
| CTACCTTGTAATTACCGCCCCATTTTCCTTTT | (SEQ ID NO: 24) |
| LONG 11b | |
| TGTCACATAAATTGACACAAAGGGAGTACTTG | (SEQ ID NO: 25) |

The primers used for amplification of the promoter, terminator and coding regions for RB and RGA3 genes were:

| | |
|---|---|
| LONG 12a | |
| AAGCGGAAGGAATGGTTTGGGTATGATAAAAT | (SEQ ID NO: 26) |
| LONG 12b | |
| CTTTCAAAATGGGAAAGGAGATTAAAGGTGGA, | (SEQ ID NO: 27) |
| and | |
| LONG 13a | |
| CCTTGTAATTGCTACCCCATTAGTATGAAAGGA | (SEQ ID NO: 28) |
| LONG 13b | |
| AGGAGATTAAAGGTGGATAATCAAACCTCACC | (SEQ ID NO: 29) |

The primers used for amplification of the promoter, terminator and coding regions for gene RGA1 gene were:

| | |
|---|---|
| LONG 14a | |
| GTTTCTTTTCCATCTTTGCCCTTTTCTTTAGG | (SEQ ID NO: 30) |
| LONG 14b | |
| ATCAATTTTATCATACCCAAACCATTCCTTCC | (SEQ ID NO: 31) |

The primers used for amplification of the promoter, terminator and coding regions for RGA4 gene were:

(SEQ ID NO: 32)

| | |
|---|---|
| LONG 15a | TTGACTTGAGTACAACAAATCCAAGTATAAAGAAG |

(SEQ ID NO: 33)

| | |
|---|---|
| LONG 15b | GTGAAGGCAAGTTTAGAAAATGGTCATGATAC |

The primers used amplification of the promoter, terminator and coding regions for gene RGA3 gene were:

| | |
|---|---|
| LONG 16a | +TL,22 |
| TGATTATAACAGACACTCAATTAGCAAGCCTGTG | (SEQ ID NO: 34) |
| LONG 16b | |
| CTTTCAAAATGGGAAAGGAGATTAAAGGTGGA | (SEQ ID NO: 27) |

The primers used for amplification of the promoter, terminator and coding regions for RB gene were:

| | |
|---|---|
| LONG 17a | |
| ATAAAGAAAAGAACTCAAAGCGGAAGGAATGG | (SEQ D NO: 35) |
| LONG 17b | |
| TGTCACATAAATTGACACAAAGGGAGTACTTGT | (SEQ ID NO: 25) |

### Example 3

The following example describes potato transformation with one of the isolated genes:

Long range PCR product corresponding to the RB gene was cloned into the binary vector pCLD04541 (Jones et al. Transgenic Research 1 285:297 (1992)). This was mobilized into Agrobacterium tumefaciens LBA4404 for plant transformation. Internodes were taken from three to four week old in vitro potato plants cv Katahdin maintained on PROP medium (Haberlach et al, (1985) Plant Sci Lett 39:67-74). Explants were placed in a suspension of Agrobacterium (4-6×10 8 cells/ml) for 30 min, blotted and transferred to ZIG medium (Clearly, 1997. Am Pot Journal 74:125-129) for a 4 day cocultivation. Internodes were then moved to ZIG medium containing 50 mg/L kanamycin to select for transformants and 250 ml/L cefataxine to suppress growth of Agrobacterium. Putative transgenic plantlets were removed from explant pieces 10 to 16 weeks later and rooted on PROP medium. DNA was extracted following established protocols.

### Example 4

The following example describes a method of late blight resistance screening:

Plants were sprayed to run-off with a fine mist of Phytophthora infestans sporangial suspension prepared from US-8, Type A2, Cornell standard ME 93-A2 (WEF#US930287) cultures maintained on rye A medium in a greenhouse facility. The suspension contained approximately 30000 sporangia/ml and was pre-chilled 4 h at 10° C. before use. Relative humidity in the greenhouse was maintained at or above 90%. The temperature was maintained at 23° C. during daylight hours (15 h) and dropped to 15° C. at night. Foliage blight scores were recorded 69, 92, 116, and 163 hours after inoculation. A blight scale, with 0 indicating a dead plant and 9 no visible infection, was used to visually rate disease severity. All the plants were tested in three repetitions. The ratings and the ranges of percentage infections associated with the rating value were as follows: 9, no visible infection; 8, less than 10% infection; 7, 11-25% infection; 6, 26-40% infection; 5, 41 to 60% infection; 4, 61-70% infection; 3, 71-80% infection; 2, 81-90% infection; 1, greater than 90% infection; 0, all dead. Plants with scores of 8 or above were scored as resistant and plants with scores of 6.9 or below were scored as susceptible. Plants with scores between 6.9 and 8 were scored as intermediate resistant.

Transgenic plants with the RB gene were tested using the above method. 69, 92, 116, and 163 hours after inoculation, the average resistant score for transgenic plants with RB gene was 7.3.

### Example 5

The following example shows an amino acid comparison between the RB gene coding region from a disease resistant and disease susceptible variety. The top sequence is the rb amino acid sequence from the susceptible 177O13 homolog (SEQ ID NO: 18). The bottom sequence is the RB amino acid sequence from the resistant homolog (SEQ ID NO:8). Note that on the line numbered 451, at residue 454, a single nucleotide difference between R and S changes a Tyrosine (Y; resistant) to an amber stop codon.

| | . . . . . | |
|---|---|---|
| 1 | MAEAFIQVLLDNLTSFLKGELALLFGFQDEFQRLSSMFSTIQAVLEDAQE | 50 |
| | \|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\| | |
| 1 | MAEAFIQVLLDNLTSFLKGELVLLFGFQDEFQRLSSMFSTIQAVLEDAQE | 50 |
| | . . . . . | |
| 51 | KQLNNKPLENWLQKLNAATYEVDDILDEYKTKATRFSQSEYGRYHPKVIP | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | KQLNNKPLENWLQKLNAATYEVDDILDEYKTKATRFSQSEYGRYHPKVIP | 100 |
| | . . . . . | |
| 101 | FRHKVGKRMDQVMKKLKAIAEERKNFHLHEKIVERQAVRRETGSVLTEPQ | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | FRHKVGKRMDQVMKKLKAIAEERKNFHLHEKIVERQAVRRETGSVLTEPQ | 150 |
| | . . . . . | |
| 151 | VYGRDKEKDEIVKILINNVSDAQHLSVLPILGMGGLGKTTLAQMVFNDQR | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | VYGRDKEKDEIVKILINNVSDAQHLSVLPILGMGGLGKTTLAQMVFNDQR | 200 |
| | . . . . . | |
| 201 | VTEHFHSKIWICVSEDFDEKRLIKAIVESIEGRPLLGEMDLAPLQKKLQE | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | VTEHFHSKIWICVSEDFDEKRLIKAIVESIEGRPLLGEMDLAPLQKKLQE | 250 |
| | . . . . . | |
| 251 | LLNGKRYLLVLDDVWNEDQQKWANLRAVLKVGASGASVLTTTRLEKVGSI | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | LLNGKRYLLVLDDVWNEDQQKWANLRAVLKVGASGASVLTTTRLEKVGSI | 300 |
| | . . . . . | |
| 301 | MGTLQPYELSNLSQEDCWLLFMQRAFGHQEEINPNLVAIGKEIVKKSGGV | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | MGTLQPYELSNLSQEDCWLLFMQPAFGHQEEINPNLVAIGKEIVKKSGGV | 350 |
| | . . . . . | |
| 351 | PLAAKTLGGILCFKREERAWEHVRDSPIWNLPQDESSILPALRLSYHQLP | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 351 | PLAAKTLGGILCFKREERAWEHVRDSPIWNLPQDESSILPALRLSYHQLP | 400 |
| | . . . . . | |
| 401 | LDLKQCFAYCAVFPKDAKMEKEKLISLWMAHGFLLSKGNMELEDVGDEVW | 450 |
| | \|\|\|\|\|\|\|\|\|\|\|\|\|\|\|\|\|\|\|.\|\|\|\|\|\|\|\|\|\|\|\|\|\|\|\|\|\|\|\|\|\|\|\|\|\|\|\|\|\| | |
| 401 | LDLKQCFAYCAVFPKDAKMKKEKLISLWMAHGFLLSKGNMELEDVGDEVW | 450 |
| | . . . . . | |
| 451 | KEL*LRSFFQEIEVKDGKTYFKMHDLIHDLATSLFSANTSSSNIREINKH | 500 |
| | \|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 451 | KELYLRSFFQEIEVKDGKTYFKMHDLIHDLATSLFSANTSSSNIREINKH | 500 |
| | . . . . . | |
| 501 | SYTHMMSIGFAEVVFFYTLPPLEKFISLRVLNLGDSTFNKLPSSIGDLVH | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 501 | SYTHMMSIGFAEVVFFYTLPPLEKFISLRVLNLGDSTFNKLPSSIGDLVH | 550 |
| | . . . . . | |
| 551 | LRYLNLYGSGMRSLPKQLCKLQNLQTLDLQYCTKLCCLPKETSKLGSLRN | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 551 | LRYLNLYGSGMRSLPKQLCKLQNLQTLDLQYCTKLCCLPKETSKLGSLRN | 600 |
| | . . . . . | |
| 601 | LLLDGSQSLTCMPPRIGSLTCLKTLGQFVVGRKKGYQLGELGNLNLYGSI | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 601 | LLLDGSQSLTCMPPRIGSLTCLKTLGQFVVGRKKGYQLGELGNLNLYGSI | 650 |
| | . . . . . | |
| 651 | KISHLERVKNDKDAKEANLSAKGNLHSLSMSWNNFGPHIYESEEVKVLEA | 700 |
| | \|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\| | |
| 651 | KISHLERVKNDMDAKEANLSAKGNLHSLSMSWNNFGPHIYESEEVKVLEA | 700 |
| | . . . . . | |
| 701 | LKPHSNLTSLKIYGFRGIHLPEWMNHSVLKNIVSILISNFRNCSCLPPFG | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 701 | LKPHSNLTSLKIYGFRGIHLPEWMNHSVLKNIVSILISNFRNCSCLPPFG | 750 |
| | . . . . . | |
| 751 | DLPCLESLELHWGSADVEYVEEVDIDVHSGFPTRIRFPSLRKLDIWDFGS | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 751 | DLPCLESLELHWGSADVEYVEEVDIDVHSGFPTRIRFPSLRKLDIWDFGS | 800 |
| | . . . . . | |
| 801 | LKGLLKKEGEEQFPVLEEMIIHECPFLTLSSNLRALTSLRICYNKVATSF | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 801 | LKGLLKKEGEEQFPVLEEMIIHECPFLTLSSNLRALTSLRICYNKVATSF | 850 |
| | . . . . . | |
| 851 | PEEMFKNLANLKYLTISRCNNLKELPTSLASLNALKSL......ALESLP | 894 |
| | \|\|\|\|\|\|\|\|\|\|\|\|\|\|\|\|\|\|\|\|\|\|\|\|\|\|\|\|\|\|\|\|\|\|\|\|\|\| \|\|\|\|\|\| | |
| 851 | PEEMFKNLANLKYLTISRCNNLKELPTSLASLNALKSLKIQLCCALESLP | 900 |
| | . . . . . | |
| 895 | EEGLEGLSSLTELFVEHCNMLKCLPEGLQHLTTLTSLKIRGCPQLIKRCE | 944 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 901 | EEGLEGLSSLTELFVEHCNMLKCLPEGLQHLTTLTSLKIRGCPQLIKRCE | 950 |
| | . . . . . | |
| 945 | KGIGEDWHKISHIPNVNIYI* | 965 |
| | \|\|\|\|\|\|\|\|\|\|\|\|\|\|\|\|\|\|\|\|\| | |
| 951 | KGIGEDWHKISHIPNVNIYI* | 971 |

### Example 6

The following example shows a nucleic acid comparison between the RB coding regions from a disease resistant and disease susceptible variety. The top sequence is the RB coding region from the resistant homolog (SEQ ID NO:7). The bottom sequence is the gene rb coding region from the susceptible 177O13 homolog (SEQ ID NO: 17). Note that the susceptible homolog contains a C to G point mutation at position 1362 that creates a stop codon in second exon at Tyr454 (residue 454 of 970 total), creating a severely truncated protein. Other than this stop codon within rb, the amino acid sequences are highly similar, with only three synonymous point mutations (C²⁸ to T, T²⁶³⁵ to C, and A²⁷⁴⁵ to G), a point mutation of T⁶⁵ to C that changes valine to alanine, and a deletion of an 18-bp sequence that results in the loss of six amino acids (KIQLCC) in the 18^{th} LRR repeat.

| | | |
|---|---|---|
| 1 | ATGGCTGAAGCTTTCATTCAAGTTCTGCTAGACAATCTCACTTCTTTCCT | 50 |
| | \|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\| | |
| 2895 | ATGGCTGAAGCTTTCATTCAAGTTCTGTTAGACAATCTCACTTCTTTCCT | 2846 |
| | . . . . . | |
| 51 | CAAAGGGGAACTTGTATTGCTTTTCGGTTTTCAAGATGAGTTCCAAAGGC | 100 |
| | \|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\| | |
| 2845 | CaAAGGGGAACTTGCATTGCTTTTCGGTTTTCAAGATGAGTTCCAAAGGC | 2796 |
| | . . . . . | |
| 101 | TTTCAAGCATGTTTTCTACAATTCAAGCCGTCCTTGAAGATGCTCAGGAG | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2795 | TTTCAAGCATGTTTTCTACAATTCAAGCCGTCCTTGAAGATGCTCAGGAG | 2746 |
| | . . . . . | |
| 151 | AAGCAACTCAACAACAAGCCTCTAGAAAATTGGTTGCAAAAACTCAATGC | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2745 | AAGCAACTCAACAACAAGCCTCTAGAAAATTGGTTGCAAAAACTCAATGC | 2696 |
| | . . . . . | |
| 201 | TGCTACATATGAAGTCGATGACATCTTGGATGAATATAAAACCAAGGCCA | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2695 | TGCTACATATGAAGTCGATGACATCTTGGATGAATATAAAACCAAGGCCA | 2646 |
| | . . . . . | |
| 251 | CAAGATTCTCCCAGTCTGAATATGGCCGTTATCATCCAAAGGTTATCCCT | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2645 | CAAGATTCTCCCAGTCTGAATATGGCCGTTATCATCCAAAGGTTATCCCT | 2596 |
| | . . . . . | |
| 301 | TTCCGTCACAAGGTCGGGAAAAGGATGGACCAAGTGATGAAAAAACTAAA | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2595 | TTCCGTCACAAGGTCGGGAAAAGGATGGACCAAGTGATGAAAAAACTAAA | 2546 |
| | . . . . . | |
| 351 | GGCAATTGCTGAGGAAAGAAAGAATTTTCATTTGCACGAAAAAATTGTAG | 400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2545 | GGCAATTGCTGAGGAAAGAAAGAATTTTCATTTGCACGAAAAAATTGTAG | 2496 |
| | . . . . . | |
| 401 | AGAGACAAGCTGTTAGACGGGAAACAGGTTCTGTATTAACCGAACCGCAG | 450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2495 | AGAGACAAGCTGTTAGACGGGAAACAGGTTCTGTATTAACCGAACCGCAG | 2446 |
| | . . . . . | |
| 451 | GTTTATGGAAGAGACAAAGAGAAAGATGAGATAGTGAAAATCCTAATAAA | 500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2445 | GTTTATGGAAGAGACAAAGAGAAAGATGAGATAGTGAAAATCCTAATAAA | 2396 |
| | . . . . . | |
| 501 | CAATGTTAGTGATGCCCAACACCTTTCAGTCCTCCCAATACTTGGTATGG | 550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2395 | CAATGTTAGTGATGCCCAACACCTTTCAGTCCTCCCAATACTTGGTATGG | 2346 |
| | . . . . . | |
| 551 | GGGGATTAGGAAAAACGACTCTTGCCCAAATGGTCTTCAATGACGAGAGA | 600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2345 | GGGGATTAGGAAAAACGACTCTTGCCCAAATGGTCTTCAATGACCAGAGA | 2296 |
| | . . . . . | |
| 601 | GTTACTGAGCATTTCCATTCCAAAATATGGATTTGTGTCTCGGAAGATTT | 650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2295 | GTTACTGAGCATTTCCATTCCAAAATATGGATTTGTGTCTCGGAAGATTT | 2246 |
| | . . . . . | |
| 651 | TGATGAGAAGAGGTTAATAAAGGCAATTGTAGAATCTATTGAAGGAAGGC | 700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2245 | TGATGAGAAGAGGTTAATAAAGGCAATTGTAGAATCTATTGAAGGAAGGC | 2196 |
| | . . . . . | |
| 701 | CACTACTTGGTGAGATGGACTTGGCTCCACTTCAAAAGAAGCTTCAGGAG | 750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2195 | CACTACTTGGTGAGATGGACTTGGCTCCACTTCAAAAGAAGCTTCAGGAG | 2146 |
| | . . . . . | |
| 751 | TTGCTGAATGGAAAAAGATACTTGCTTGTCTTAGATGATGTTTGGAATGA | 800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2145 | TTGCTGAATGGAAAAAGATACTTGCTTGTCTTAGATGATGTTTGGAATGA | 2096 |
| | . . . . . | |
| 801 | AGATCAACAGAAGTGGGCTAATTTAAGAGCAGTCTTGAAGGTTGGAGCAA | 850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2095 | AGATCAACAGAAGTGGGCTAATTTAAGAGCAGTCTTGAAGGTTGGAGCAA | 2046 |
| | . . . . . | |
| 851 | GTGGTGCTTCTGTTCTAACCACTACTCGTCTTGAAAAGGTTGGATCAATT | 900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 2045 | GTGGTGCTTCTGTTCTAACCACTACTCGTCTTGAAAAGGTTGGATCAATT | 1996 |
| | . . . . . | |
| 901 | ATGGGAACATTGCAACCATATGAACTGTCAAACCTGTCTCAACAAGATTG | 950 |
| | \|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\| | |
| 1995 | ATGGGAACATTGCAACCATATGAACTGTCAAATCTGTCTCAAGAAGATTG | 1946 |
| | . . . . . | |
| 951 | TTGGTTGTTGTTCATGCAACGTGCATTTGGACACCAAGAAGAAATAAATC | 1000 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1945 | TTGGTTGTTGTTCATGCAACGTGCATTTGGACACCAAGAAGAAATAAATC | 1896 |
| | . . . . . | |
| 1001 | CAAACCTTGTGGCAATCGGAAAGGAGATTGTGAAAAAAAGTGGTGGTGTG | 1050 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1895 | CAAACCTTGTGGCAATCGGAAAGGAGATTGTGAAAAAAAGTGGTGGTGTG | 1846 |
| | . . . . . | |
| 1051 | CCTCTAGCAGCCAAAACTCTTGGAGGTATTTTGTGCTTCAAGAGAGAAGA | 1100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1845 | CCTCTAGCAGCCAAAACTCTTGGAGGTATTTTGTGCTTCAAGAGAGAAGA | 1796 |
| | . . . . . | |
| 1101 | AAGAGCATGGGAACATGTGAGAGACAGTCCGATTTGGAATTTGCCTCAAG | 1150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1795 | AAGAGCATGGGAACATGTGAGAGACAGTCCGATTTGGAATTTGCCTCAAG | 1746 |
| | . . . . . | |
| 1151 | ATGAAAGTTCTATTCTGCCTGCCCTGAGGCTTAGTTACCATCAACTTCCA | 1200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1745 | ATGAAAGTTCTATTCTGCCTGCCCTGAGGCTTAGTTACCATCAACTTCCA | 1696 |
| | . . . . . | |
| 1201 | CTTGATTTGAAACAATGCTTTGCGTATTGTGCGGTGTTCCCAAAGGATGC | 1250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1695 | CTTGATTTGAAACAATGCTTTGCGTATTGTGCGGTGTTCCCAAAGGATGC | 1646 |
| | . . . . . | |
| 1251 | CAAAATGAAAAAAGAAAAGCTAATCTCTCTCTGGATGGCGCATGGTTTTC | 1300 |
| | \|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\| | |
| 1645 | CAAAATGGAAAAAGAAAAGCTAATCTCTCTCTGGATGGCGCATGGTTTTC | 1596 |
| | . . . . . | |
| 1301 | TTTTATCAAAAGGAAACATGGAGCTAGAGGATGTGGGCGATGAAGTATGG | 1350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1595 | TTTTATCAAAAGGAAACATGGAGCTAGAGGATGTGGGCGATGAAGTATGG | 1546 |
| | . . . . . | |
| 1351 | AAAGAATTATACTTGAGGTCTTTTTTCCAAGAGATTGAAGTTAAAGATGG | 1400 |
| | \|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\| | |
| 1545 | AAAGAATTATAGTTGAGGTCTTTTTTCCAAGAGATTGAAGTTAAAGATGG | 1496 |
| | . . . . . | |
| 1401 | TAAAACTTATTTCAAGATGCATGATCTCATCCATGATTTGGCAACATCTC | 1450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1495 | TAAAACTTATTTCAAGATGCATGATCTCATCCATGATTTGGCAACATCTC | 1446 |
| | . . . . . | |
| 1451 | TGTTTTCAGCAAACACATCAAGCAGCAATATCCGTGAAATAAATAAACAC | 1500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1445 | TGTTTTCAGCAAACACATCAAGCAGCAATATCCGTGAAATAAATAAACAC | 1396 |
| | . . . . . | |
| 1501 | AGTTACACACATATGATGTCCATTGGTTTCGCCGAAGTGGTGTTTTTTTA | 1550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1395 | AGTTACACACATATGATGTCCATTGGTTTCGCCGAAGTGGTGTTTTTTTA | 1346 |
| | . . . . . | |
| 1551 | CACTCTTCCCCCCTTGGAAAAGTTTATCTCGTTAAGAGTGCTTAATCTAG | 1600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1345 | CACTCTTCCCCCCTTGGAAAAGTTTATCTCGTTAAGAGTGCTTAATCTAG | 1296 |
| | . . . . . | |
| 1601 | GTGATTCGACATTTAATAAGTTACCATCTTCCATTGGAGATCTAGTACAT | 1650 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1295 | GTGATTCGACATTTAATAAGTTACCATCTTCCATTGGAGATCTAGTACAT | 1246 |
| | . . . . . | |
| 1651 | TTAAGATACTTGAACCTGTATGGCAGTGGCATGCGTAGTCTTCCAAAGCA | 1700 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1245 | TTAAGATACTTGAACCTGTATGGCAGTGGCATGCGTAGTCTTCCAAAGCA | 1196 |
| | . . . . . | |
| 1701 | GTTATGCAAGCTTCAAAATCTGCAAACTCTTGATCTACAATATTGCACCA | 1750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1195 | GTTATGCAAGCTTCAAAATCTGCAAACTCTTGATCTACAATATTGCACCA | 1146 |
| | . . . . . | |
| 1751 | AGCTTTGTTGTTTGCCAAAAGAAACAAGTAAACTTGGTAGTCTCCGAAAT | 1800 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1145 | AGCTTTGTTGTTTGCCAAAAGAAACAAGTAAACTTGGTAGTCTCCGAAAT | 1096 |
| | . . . . . | |
| 1801 | CTTTTACTTGATGGTAGCCAGTCATTGACTTGTATGCCACCAAGGATAGG | 1850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1095 | CTTTTACTTGATGGTAGCCACTCATTGACTTGTATGCCACCAAGGATAGG | 1046 |
| | . . . . . | |
| 1851 | ATCATTGACATGCCTTAAGACTCTAGGTCAATTTGTTGTTGGAAGGAAGA | 1900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1045 | ATCATTGACATGCCTTAAGACTCTAGGTCAATTTGTTGTTGGAAGGAAGA | 996 |
| | . . . . . | |
| 1901 | AAGGTTATCAACTTGGTGAACTAGGAAACCTAAATCTCTATGGCTCAATT | 1950 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 995 | AAGGTTATCAACTTGGTGAACTAGGAAACCTAAATCTCTATGGCTCAATT | 946 |
| | . . . . . | |
| 1951 | AAAATCTCGCATCTTGAGAGAGTGAAGAATGATATGGACGCAAAAGAAGC | 2000 |
| | \|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\| | |
| 945 | AAAATCTCGCATCTTGAGAGAGTGAAGAATGATAAGGACGCAAAAGAAGC | 896 |
| | . . . . . | |
| 2001 | CAATTTATCTGCAAAAGGGAATCTGCATTCTTTAAGCATGAGTTGGAATA | 2050 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 895 | CAATTTATCTGCAAAAGGGAATCTGCATTCTTTAAGCATGAGTTGGAATA | 846 |
| | . . . . . | |
| 2051 | ACTTTGGACCACATATATATGAATCAGAAGAAGTTAAAGTGCTTGAAGCC | 2100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 845 | ACTTTGGACCACATATATATGAATCAGAAGAAGTTAAAGTGCTTGAAGCC | 796 |
| | . . . . . | |
| 2101 | CTCAAACCACACTCCAATCTGACTTCTTTAAAAATCTATGGCTTCAGAGG | 2150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 795 | CTCAAACCACACTCCAATCTGACTTCTTTAAAAATCTATGGCTTCAGAGG | 746 |
| | . . . . . | |
| 2151 | AATCCATCTCCCAGAGTGGATGAATCACTCAGTATTGAAAAATATTGTCT | 2200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 745 | AATCCATCTCCCAGAGTGGATGAATCACTCAGTATTGAAAAATATTGTCT | 696 |
| | . . . . . | |
| 2201 | CTATTCTAATTAGCAACTTCAGAAACTGCTCATGCTTACCACCCTTTGGT | 2250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 695 | CTATTCTAATTAGCAACTTCAGAAACTGCTCATGCTTACCACCCTTTGGT | 646 |
| | . . . . . | |
| 2251 | GATCTGCCTTGTCTAGAAAGTCTAGAGTTACACTGGGGGTCTGCGGATGT | 2300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 645 | GATCTGCCTTGTCTAGAAAGTCTAGAGTTACACTGGGGGTCTGCGGATGT | 596 |
| | . . . . . | |
| 2301 | GGAGTATGTTGAAGAAGTGGATATTGATGTTCATTCTGGATTCCCCACAA | 2350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 595 | GGAGTATGTTGAAGAAGTGGATATTGATGTTCATTCTGGATTCCCCACAA | 546 |
| | . . . . . | |
| 2351 | GAATAAGGTTTCCATCCTTGAGGAAACTTGATATATGGGACTTTGGTAGT | 2400 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 545 | GAATAAGGTTTCCATCCTTGAGGAAACTTGATATATGGGACTTTGGTAGT | 496 |
| | . . . . . | |
| 2401 | CTGAAAGGATTGCTGAAAAAGGAAGGAGAAGAGCAATTCCCTGTGCTTGA | 2450 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 495 | CTGAAAGGATTGCTGAAAAAGGAAGGAGAAGAGCAATTCCCTGTGCTTGA | 446 |
| | . . . . . | |
| 2451 | AGAGATGATAATTCACGAGTGCCCTTTTCTGACCCTTTCTTCTAATCTTA | 2500 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 445 | AGAGATGATAATTCACGAGTGCCCTTTTCTGACCCTTTCTTCTAATCTTA | 396 |
| | . . . . . | |
| 2501 | GGGCTCTTACTTCCCTCAGAATTTGCTATAATAAAGTAGCTACTTCATTC | 2550 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 395 | GGGCTCTTACTTCCCTCAGAATTTGCTATAATAAAGTAGCTACTTCATTC | 346 |
| | . . . . . | |
| 2551 | CCAGAAGAGATGTTCAAAAACCTTGCAAATCTCAAATACTTGACAATCTC | 2600 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 345 | CCAGAAGAGATGTTCAAAAACCTTGCAAATCTCAAATACTTGACAATCTC | 296 |
| | . . . . . | |
| 2601 | TCGGTGCAATAATCTCAAAGAGCTGCCTACCAGCTTGGCTAGTCTGAATG | 2650 |
| | \|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\| | |
| 295 | TCGGTGCAATAATCTCAAAGAGCTGCCTACCAGCCTGGCTAGTCTGAATG | 246 |
| | . . . . . | |
| 2651 | CTTTGAAAAGTCTAAAAATTCAATTGTGTTGCGCACTAGAGAGTCTCCCT | 2700 |
| | \|\|\|\|\|\|\|\|\|\|\|\|\|\| \|\|\|\|\|\|\|\|\|\|\|\|\|\|\|\|\|\| | |
| 245 | CTTTGAAAAGTCTA..................GCACTAGAGAGTCTCCCT | 214 |
| | . . . . . | |
| 2701 | GAGGAAGGGCTGGAAGGTTTATCTTCACTCACAGAGTTATTTGTTGAACA | 2750 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 213 | GAGGAAGGGCTGGAAGGTTTATCTTCACTCACAGAGTTATTTGTTGAACA | 164 |
| | . . . . . | |
| 2751 | CTGTAACATGCTAAAATGTTTACCAGAGGGATTGCAGCACCTAACAACCC | 2800 |
| | \|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\| | |
| 163 | CTGTAACATGCTGAAATGTTTACCAGAGGGATTGCAGCACCTAACAACCC | 114 |
| | . . . . . | |
| 2801 | TCACAAGTTTAAAAATTCGGGGATGTCCACAACTGATCAAGCGGTGTGAG | 2850 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 113 | TCACAAGTTTAAAAATTCGGGGATGTCCACAACTGATCAAGCGGTGTGAG | 64 |
| | . . . . . | |
| 2851 | AAGGGAATAGGAGAAGACTGGCACAAAATTTCTCACATTCCTAATGTGAA | 2900 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 63 | AAGGGAATAGGAGAAGACTGGCACAAAATTTCTCACATTCCTAATGTGAA | 14 |
| | . | |
| 2901 | TATATATATTTAA | 2913 |
| | \|\|\|\|\|\|\|\|\|\|\|\|\| | |
| 13 | TATATATATTTAA | 1 |

### Example 7

RACE Analysis:

Three-week-old Solanum bulbocastinum PT29 plants were inoculated with P. infestans US8 mating type A2 and maintained in greenhouse facilities at the University of Wisconsin Biotron as described previously (Naess et al. 2000). Equal amount of unchallenged leaves of PT29 and challenged leaves 12 h, and 1, 2, 3, 4, 5 days after inoculation were collected and pooled. Total RNA was isolated from the combined materials using TRIZOL (Invitrogen, Carlsbad, Calif.). Poly(A)+ RNA was isolated using PolyATract mRNA Isolation Systems (Promega, Madison, Wis.) according to the manufacturer's instructions. The 5′ and 3′ ends of the cDNA were determined by rapid amplification of cDNA ends (RACE) using the GeneRacer Kit (Invitrogen, Carlsbad, Calif.). The RACE primers are identical between two alleles and listed in table 1. The sequences of full-length cDNA of gene genes RGA1, RB, RGA3, and RGA4 were determined by RACE analysis.

TABLE 3

| | |
|---|---|
| Primer | Sequence |

| | | |
|---|---|---|
| 1-5′-RACE | 5′-GGAGCCAAGTCCATGTCACTGAGGGA-3′ | (SEQ ID NO:109) |
| 1-3′-RACE | 5′-ATGCAATTGCTGAGGAACGAAAGAAG-3′ | (SEQ ID NO:110) |
| 2-5′-RACE | 5′-ATCCACTTCTTCAACATACTCCACATCC-3′ | (SEQ ID NO:111) |
| 2-3′-RACE-a | 5′-GAGAGTGAAGAATGATAAGGACGCAAAA-3′ | (SEQ ID NO:112) |
| 2-3′-RACE-b | 5′-GGTGTTTTTTTACACTCTTCCCCCCTTGG-3′ | (SEQ ID NO:113) |
| 2-3′-RACE-c | 5′-CCAAGGCCACAAGATTCTCCC-3′ | (SEQ ID NO:114) |
| 3-5′-RACE-a | 5′-AGGGGAGCCAAGTCCATGTCACCCAGT-3′ | (SEQ ID NO:115) |
| 3-5′-RACE-b | 5′-GTTTAGGACTTGTTTCGGTTTGGTGGCA-3′ | (SEQ ID NO:116) |
| 3-3′-RACE-a | 5′-TGCCACCAAACCGAAACAAGTCCTAAA-3′ | (SEQ ID NO:117) |
| 3-3′-RACE-b | 5′-CATCCACGGACCATCACTTTCTGTTA-3′ | (SEQ ID NO:118) |
| 4-5′-RACE | 5′-TGAAATGAAGCCAAGTCCTCAACATGAG-3′ | (SEQ ID NO:119) |
| 4-3′-RACE | 5′-AAATTACAGAGAGACAAGCTGCCGCTGC-3′ | (SEQ ID NO:120) |

### Example 8

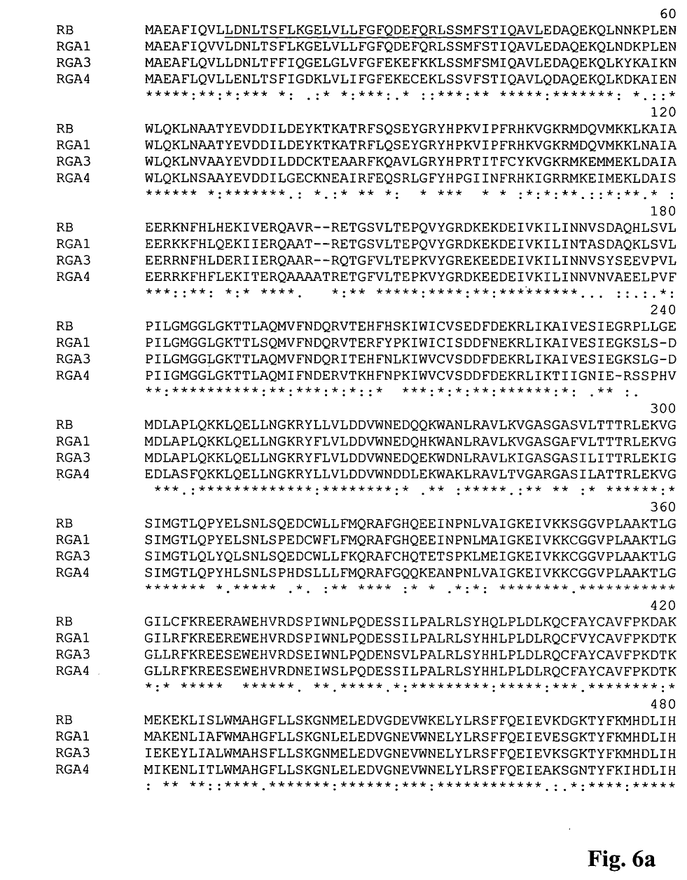

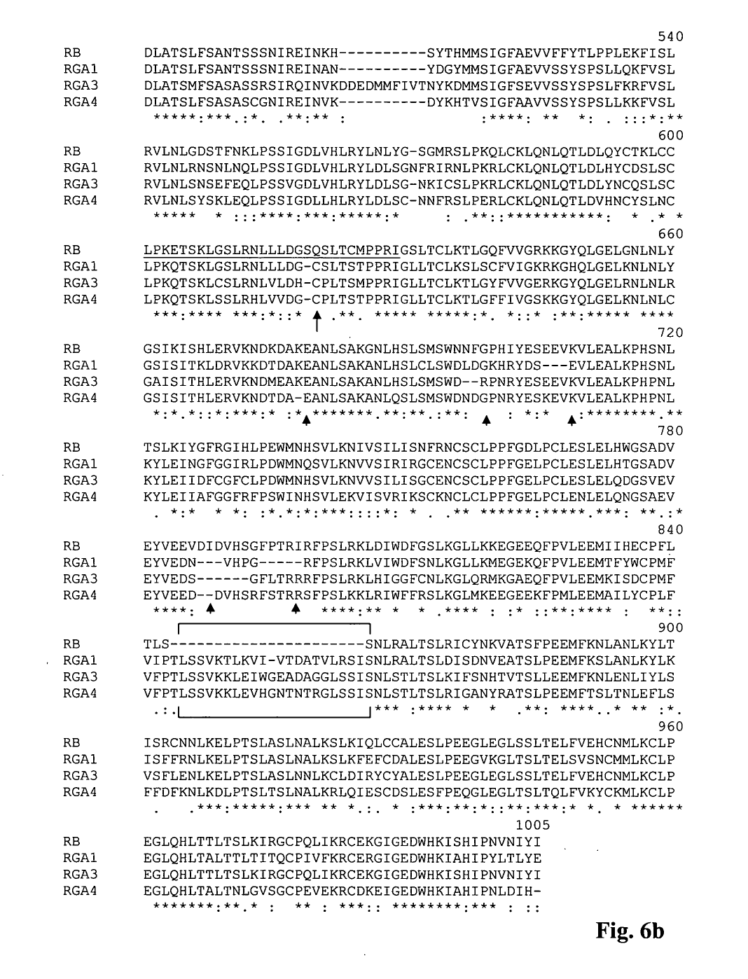

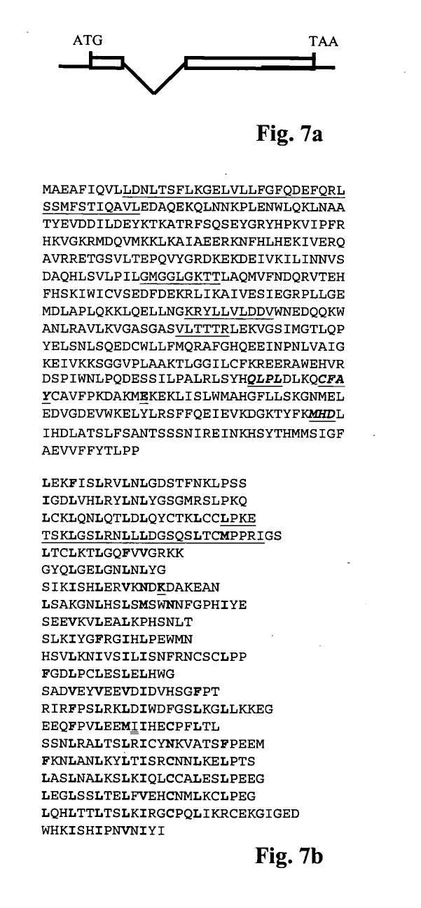

FIGS. 6A and 6B provide a comparison of the RB, RGA1, RGA3 and RGA4 protein sequences. The putative leucine zipper motif and a heptad repeat motif are underlined. Both RB and rb contain 21 LRR repeats, whereas RGA1, RGA3, and RGA4 contain 22 LRR repeats (FIG. 1). The variation of LRR repeats may play a role in determining recognition specificity of the RB protein. It has been demonstrated that expansion and contraction of LRR repeats are responsible for loss of function or recognition specificities of plant disease resistance genes. In flax, inactivation of the rust resistance gene M was associated with the loss of a single repeated unit within the LRR coding region (Anderson et al. 1997 Plant Cell 9, 641-651). Sequence analysis of mutant RPP5 alleles identified four duplicated LRR repeats in comparison to the wild-type RPP5 gene (Parker et al. 1997 Plant Cell 9, 879-894). Recently, domain swapping and gene shuffling of tomato Cf-4 and Cf-9 protein also demonstrated that variation in LRR copy number plays a major role in determining recognition specificity in these proteins (Wulff et al. 2001 Plant Cell 13: 255-272).

The RB protein belongs to the NBS-LRR class of R proteins. Its putative NBS domain consists of three motifs: kinase 1a or P-loop (positions 182-190), kinase 2 (positions 255-264), and kinase 3a (positions 288-293). Downstream of the kinase motifs is a domain conserved among resistance genes: QLPL, CFAY, and MHD motifs. The RB protein contains one putative five-heptad leucine zipper motif near the N terminus (positions 10-45). Another region containing four heptad repeats (positions 588-609) can be observed within the LRR domain. The LRR domain consists of 21 LRR repeats.

1. An isolated nucleic acid encoding a RB polypeptide, wherein the nucleic acid comprises a polynucleotide sequence at least 70% identical to a polynucleotide sequence as shown in SEQ ID NO:4 or SEQ ID NO:7, and wherein the polypeptide, when produced in a solanaceous plant, confers disease resistance in the plant.

2. The nucleic acid of claim 1, wherein the nucleic acid comprises a polynucleotide sequence at least 95% identical to a polynucleotide sequence as shown in SEQ ID NO:4 or SEQ ID NO:7.

3. The nucleic acid of claim 1, wherein the polynucleotide sequence is SEQ ID NO:4.

4. The nucleic acid of claim 1, wherein the polynucleotide sequence is SEQ ID NO:7.

5. The nucleic acid of claim 1, wherein the nucleic acid is isolated from Solanum Bulbocastanum.

6. The nucleic acid of claim 1, wherein the plant is from the Solanum species.

7. The nucleic acid of claim 6, wherein the plant is selected from the group consisting of potato, tomato and eggplant.

8. A nucleic acid of claim 1, wherein the polypeptide, when produced in a plant, confers disease resistance to an oomycete pathogen.

9. The nucleic acid of claim 8, wherein the oomycete pathogen is Phytophthora infestans.

10. An isolated nucleic acid encoding a RB polypeptide, wherein the nucleic acid encodes a polypeptide having an amino acid sequence at least 70% identical to the amino acid sequence of SEQ ID NO:8 and wherein the polypeptide, when produced in a plant, confers disease resistance in the plant.

11. The nucleic acid of claim 10, wherein the polypeptide has an amino acid sequence at least 95% identical to SEQ ID NO:8.

12. The nucleic acid of claim 10 wherein the polypeptide is SEQ ID NO:5 or SEQ ID NO:8.

13. A recombinant expression cassette comprising a promoter sequence operably linked to a nucleic acid encoding a RB polypeptide, wherein the nucleic acid encodes a polynucleotide sequence at least 70% identical to a polynucleotide sequence as shown in SEQ ID NO:4 or SEQ ID NO:7 and wherein the polypeptide, when produced in a solanaceous plant, confers disease resistance in the plant.

14. The expression cassette of claim 13, wherein the nucleic acid comprises a polynucleotide sequence at least 95% identical to a polynucleotide sequence as shown in SEQ ID NO:4 or SEQ ID NO:7.

15. The expression cassette of claim 13, wherein the polynucleotide sequence is SEQ ID NO:4.

16. The expression cassette of claim 13, wherein the polynucleotide sequence is SEQ ID NO:7.

17. The expression cassette of claim 13, wherein the nucleic acid is operably linked to the promoter sequence in an antisense orientation.

18. The expression cassette of claim 13, wherein the nucleic acid is operably linked to the promoter sequence in a sense orientation.

19. The expression cassette of claim 13, wherein the polypeptide confers disease resistance to an oomycete pathogen.

20. The expression cassette of claim 19, wherein the oomycete pathogen is Phytophthora infestans.

21. The expression cassette of claim 13, wherein the promoter is a constitutive promoter.

22. The expression cassette of claim 13, wherein the promoter is a tissue specific promoter.

23. The expression cassette of claim 13, wherein the promoter sequence is SEQ ID NO:23.

24. A host cell transformed with the expression cassette of claim 13.

25. The host cell of claim 24 wherein the host cell is a plant cell from a solanaceous plant.

26. A transgenic solanaceous plant comprising a recombinant expression cassette comprising a promoter sequence operably linked to a nucleic acid encoding a RB polypeptide, wherein the nucleic acid comprises a polynucleotide sequence at least 70% identical to a polynucleotide sequence as shown in SEQ ID NO:4 or SEQ ID NO:7, and wherein the polypeptide, when produced in the plant, confers disease resistance in the plant.

27. The transgenic plant of claim 26, wherein the nucleic acid is operably linked to the promoter sequence in an antisense orientation.

28. The transgenic plant of claim 26, wherein the nucleic acid is operably linked to the promoter sequence in a sense orientation.

29. The transgenic plant of claim 26, wherein the polypeptide confers disease resistance to an oomycete pathogen.

30. The transgenic plant of claim 26, wherein the oomycete pathogen is Phytophthora infestans.

31. The transgenic plant of claim 26, wherein the plant is from the Solanum species.

32. The transgenic plant of claim 31, wherein the plant is a potato plant.

33. An isolated RB polypeptide comprising an amino acid sequence at least 70% identical to SEQ ID NO:8 and wherein the polypeptide, when produced in a solanaceous plant, confers disease resistance in the plant.

34. The polypeptide of claim 33, wherein the amino acid sequence is SEQ ID NO:5.

35. The polypeptide of claim 33, wherein the amino acid sequence is SEQ ID NO:8.

36. An antibody immunologically specific for the polypeptide of claim 33.

37. The antibodies of claim 36, immunologically specific for an amino-terminal portion of a polypeptide of claim 36.

38. A method of enhancing disease resistance in a solanaceous plant, the method comprising introducing a construct comprising a promoter operably linked to a nucleic acid encoding a RB polypeptide wherein the nucleic acid comprises a polynucleotide sequence at least 70% identical to a polynucleotide sequence as shown in SEQ ID NO:4 or SEQ ID NO:7, and wherein the polypeptide, when produced in a plant, confers disease resistance in the plant.

39. The method of claim 38, wherein the nucleic acid comprises a polynucleotide sequence at least 95% identical to a polynucleotide sequence as shown in SEQ ID NO:4 or SEQ ID NO:7.

40. The method of claim 38, wherein the polynucleotide sequence is at SEQ ID NO:4.

41. The method of claim 38, wherein the polynucleotide sequence is at SEQ ID NO:7.

42. The method of claim 38, wherein the promoter is SEQ ID NO:23.

43. The method of claim 38, wherein the method enhances disease resistance to an oomycete pathogen.

44. The method of claim 43, wherein the oomycete pathogen is Phytophthora infestans.

45. The method of claim 38, the method further comprising selecting a plant with a phenotype associated with increased disease resistance.

46. A kit for enhancing disease resistance in a solanaceous plant, the kit comprising a construct comprising a promoter operably linked to a nucleic acid encoding a RB polypeptide wherein the nucleic acid comprises a polynucleotide sequence at least 70% identical to a polynucleotide sequence as shown in SEQ ID NO:4 or SEQ ID NO:7, and wherein the polypeptide, when produced in a plant, confers disease resistance in the plant.

47. The nucleic acid of claim 1 wherein the polynucleotide is labeled.

48. An isolated nucleic acid comprises a polynucleotide sequence which hybridizes under stringent conditions to SEQ ID NO:4 or SEQ ID NO:7 or the complement thereof, wherein said nucleic acid encodes a RB polypeptide and wherein the polypeptide, when produced in a solanaceous plant, confers disease resistance in the plant wherein the hybridization reaction is incubated at 42° C. in a solution comprising 50% formamide, 5×SSC, and 1% SDS or at 65° C. in a solution comprising 5×SSC and 1% SDS, with a wash in 0.2×SSC and 0.1% SDS at 65° C.

49. The nucleic acid of claim 48, wherein the plant is selected from the group consisting of potato, tomato and eggplant.

50. A nucleic acid of claim 48, wherein the polypeptide, when expressed in a plant, confers disease resistance to an oomycete pathogen.

51. An isolated nucleic acid molecule for controlling expression of genes in transformed plant cells, which comprises a segment of a RB gene from a plant species selected from the Solanaceae species, the segment commencing at a location about 2,500 bases upstream from a transcription initiation site of the gene, and ending at a location about 250 bases downstream from the transcription initiation site.

52. The nucleic acid molecule of claim 51, wherein the plant is a potato plant.

53. The nucleic acid molecule of claim 51, isolated from a gene having a coding sequence at least 70% identical to SEQ ID NO:7.

54. A fragment of the nucleic acid molecule of claim 51, comprising a segment commencing at about 2,500 bases upstream from the transcription initiation site and terminating about 25 bases downstream from the transcription initiation site.

55. The nucleic acid molecule of claim 51, isolated from a gene having a coding sequence coding sequence at least 70% identical to SEQ ID NO:7.

56. A DNA segment for effecting expression of coding sequences operably linked to the segment, isolated from a RB gene whose coding region hybridizes under stringent conditions with a coding region defined by SEQ ID NO:7, the segment comprising a promoter and a transcription initiation site.

57. The DNA segment of claim 56, which further comprises an element that confers disease resistance on expression of the coding sequences.

58. The DNA segment of claim 56, isolated from S. Bulbocastanum.

59. A DNA segment for modulating expression of coding sequences operably linked to the segment, isolated from a gene whose coding region hybridizes under stringent conditions with a coding region defined by SEQ ID NO:7, the segment comprising a polyadenylation signal.

60. The DNA segment of claim 59, isolated from S. Bulbocastanum.

61. An expression cassette comprising the nucleic acid molecule of claim 51 operably linked to a nucleic acid encoding a polypeptide, wherein the nucleic acid encodes a polynucleotide sequence at least 70% identical to a polynucleotide sequence as shown in SEQ ID NO:4 or SEQ ID NO:7.

62. A cell transformed with the expression cassette of claim 61.

63. The transformed cell of claim 62, which is a potato plant cell.

64. A transgenic potato plant produced by regenerating the transformed plant cell of claim 63.

65. A reproductive unit of the transgenic plant of claim 64.
